(19) United States
(12) Patent Application Publication
(10) Pub. No.: US 2025/0028106 A1
(43) Pub. Date: Jan. 23, 2025

(54) Single Implantable Fiber Device for 2D Dynamic and Reconfigurable Light Emission and Collection

(71) Applicant: Washington University, St. Louis, MO (US)

(72) Inventors: Song Hu, St. Louis, MO (US); Shuo Yang, St. Louis, MO (US)

(73) Assignee: Washington University, St. Louis, MO (US)

(21) Appl. No.: 18/780,463

(22) Filed: Jul. 22, 2024

Related U.S. Application Data
(60) Provisional application No. 63/514,859, filed on Jul. 21, 2023.

(51) Int. Cl. F21V 8/00; A61B 18/22; G02B 6/02

(52) U.S. Cl. CPC G02B 6/0006; A61B 18/22; G02B 6/02042; G02B 6/02147; A61B 2018/2205

(57) ABSTRACT

Among the various aspects of the present disclosure is the provision of systems and methods for light-based procedures that include a fiber-optic bi-directional interface device that includes a multi-core optical fiber comprising a plurality of light-guiding cores in which a first portion of the plurality of light-guiding cores each comprise a light emission modification configured to direct light propagating along the light-guiding core in a laterally outward or sideways direction relative to the propagation axis of the light-guiding core; and a second portion of the plurality of light-guiding cores each further comprises a light collection modification configured to receive light produced by a source positioned laterally outwards or sideways relative to the propagation axis of the light-guiding core.

### CROSS-REFERENCE TO RELATED APPLICATIONS

This application claims priority from U.S. Provisional Application Ser. No. 63/514,859 filed on Jul. 21, 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to implantable fiber-optic devices, systems, and methods of use thereof.

### BACKGROUND OF THE INVENTION

Because of the high spatial resolution and superb molecular sensitivity and specificity, light-based imaging, sensing, and manipulation are widely used in biomedicine. However, the limited tissue penetration of light, in comparison to non-optical approaches such as ultrasound and magnetic resonance, has been a major limiting factor for its practical applications, especially in clinical translation.

To address this limitation, fiber-optic techniques have been developed to facilitate minimally invasive access to deep tissues in animals and humans that have found many promising applications, including but not limited to fiber photometry, optogenetic stimulation, micro-endoscopy, sensing, and laser ablation surgery. However, existing fiber-optic techniques typically rely on the emission and collection of light at the fiber tip, which limits the spatial coverage of a single fiber implant to a very small tissue volume near the fiber tip. To expand the spatial coverage, multiple fiber implants are usually required that inevitably increase the invasiveness and complexity of the procedure. Furthermore, due to the limited number of fibers that can be implanted in a subject, multiple subjects are often needed for comprehensive studies, which may introduce confounding factors originating from inter-subject and/or inter-procedure variations. Recently, tapered optical fibers have been adopted to extend light emission and collection from the fiber tip to a few sparsely distributed locations along the fiber axis. However, with a single implant, this technique is limited to 1D coverage along the fiber axis and suffers from relatively poor spatial resolution and length coverage, which significantly limits its applicability in animal research and its clinical translatability.

### SUMMARY OF THE INVENTION

Among the various aspects of the present disclosure is the provision of systems and methods for use in light-based procedures including optogenetic stimulation and photometry.

In one aspect, a fiber-optic bi-directional interface device is disclosed that includes a multi-core optical fiber comprising a plurality of light-guiding cores, wherein each light-guiding core comprises opposed proximal and distal ends, and the proximal end is configured to receive light from a light source. A first portion of the plurality of light-guiding cores further comprises light emission modifications configured to direct light propagating distally along the light-guiding cores in a laterally outward or sideways direction relative to the propagation axis of the light-guiding core. A second portion of the plurality of light-guiding cores further comprises light collection modifications configured to receive light produced by a source positioned laterally outwards or sideways relative to the propagation axis of the light-guiding core and direct the received light proximally. In some aspects, the light emission modifications and the light collection modifications are distributed along an emission/collection span extending a predetermined proximal-distal distance along the light-guiding cores. In some aspects, the light emission modifications are selected from the group consisting of photonic crystals, fiber Bragg gratings (FNGs), and any combination thereof. In some aspects, the light collection modifications are selected from the group consisting of total internal reflection (TIR) mirrors, multi-layer gradient filters, and any combination thereof. In some aspects, the device further includes a light trap positioned at the distal ends of the plurality of light-guiding cores,

In another aspect, a system to conduct a light-based procedure is disclosed in which the system includes a fiber-optic bi-directional interface device optically coupled to an interrogation assembly. The fiber-optic bi-directional interface device comprises a multi-core optical fiber comprising a plurality of light-guiding cores, in which each light-guiding core comprises opposed proximal and distal ends and the proximal end is configured to receive light from a light source. A first portion of the plurality of light-guiding cores further comprises light emission modifications configured to direct light propagating distally along the light-guiding cores in a laterally outward or sideways direction relative to the propagation axis of the light-guiding core. A second portion of the plurality of light-guiding cores further comprises light collection modifications configured to receive light produced by a source positioned laterally outwards or sideways relative to the propagation axis of the light-guiding core and direct the received light proximally. The interrogation assembly comprises the light source optically coupled to the proximal ends of the plurality of light-guiding cores and a light detector optically coupled to the proximal ends of the plurality of light-guiding cores. The light source is configured to produce and direct light selectively into the proximal end of one or more light-guiding cores from the first plurality in a pre-determined pattern. The light detector is configured to receive and detect light propagating from the proximal end of one or more light-guiding cores from the second plurality. In some aspects, the light emission modifications and the light collection modifications are distributed along an emission/collection span extending a predetermined proximal-distal distance along the light-guiding cores. In some aspects, the light emission modifications are selected from the group consisting of photonic crystals, fiber Bragg gratings (FNGs), and any combination thereof. In some aspects, the light collection modifications are selected from the group consisting of total internal reflection (TIR) mirrors, multi-layer gradient filters, and any combination thereof. In some aspects, the system further includes a light trap positioned at the distal ends of the plurality of light-guiding cores. In some aspects, the system further includes a launching element configured to selectively transmit a portion of light produced by the light source into the proximal ends of one or more light-guiding cores in the predetermined pattern. The launching element may include one of a spatial light modulator optically coupled between the light source and the proximal ends of the plurality of light-guiding cores; a digital mirror device (DMD) optically coupled between the light source and the proximal ends of the plurality of light-guiding cores; a galvo scanner operatively coupled to the light source to scan the light source point by point to the one or more light-guiding core; or a MEMs mirror optically coupled between the light source and the proximal ends of the plurality of light-guiding cores. In some aspects, the pre-determined pattern is selected from a depth-selective light pattern, a spatially patterned light pattern, a large-volume illumination light pattern, and any combination thereof. In some aspects, the light-based procedure selected from the optogenetic stimulation, photometry, microscopic imaging, tomographic imaging, and any combination thereof.

In another aspect, a method of producing a fiber-optic bi-directional interface device configured to transmit and receive light oriented perpendicular to a device light propagation axis is disclosed. The method includes providing a multi-core optical fiber comprising a plurality of light-guiding cores. Each light-guiding core comprising opposed proximal and distal ends. For a first portion of the light-guiding cores, the method further includes delivering a series of slit-shaped laser pulses from a femto laser to a selected interior region of the light-guiding core to produce a series of cavities within a proximal-distal section of the light-guiding core to form a fiber grating within the fiber, wherein the fiber grating is configured to direct light propagating distally along the light-guiding cores in a laterally outward or sideways direction relative to the propagation axis of the light-guiding core. For a second portion of the plurality of light-guiding cores, the method further includes delivering a series of laser pulses from a femto laser to a selected interior region of the light-guiding core to produce a rectangular-shaped cavity within a proximal-distal section of the light-guiding core to form a total internal reflection (TIR) mirror configured to receive light produced by a source positioned laterally outwards or sideways relative to the propagation axis of the light-guiding core and direct the received light proximally along the light-guiding core.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic of an example coronal section of mouse SC with current functional map overlapped.

FIG. 2 is a set of images of selective emission profiles for the fiber-optic light emitter prototype visualized in fluorescein solution. Scale bar: 500 μm.

FIG. 3 is a graph of an averaged time course (n=3) of locomotion speed before, during, and after activation at the intermediate SC layer by the fiber-optic emitter prototype in freely moving mice.

FIG. 4 is a schematic showing divergent pathways for dimorphic defensive behavior.

FIG. 5A is a schematic of the working principle of the bi-directional fiber-optic.

FIG. 5B is a schematic of the working principle of the bi-directional fiber-optic used for optogenetics.

FIG. 5C is a schematic of the working principle of the bi-directional fiber-optic used for photometry.

FIG. 6 is a schematic of the interrogation system for the bi-directional fiber-optic neural interface. Product information: Laser1: LRS-0561, Laserglow; HW: WPHSM05-561, Thorlabs; L1: LC1060-A-ML, Thorlabs; L2: LA 1509-A, Thorlabs; SLM: EXULUS-HD2, Thorlabs; L3: ACT508-300-A-ML, Thorlabs; PH: ID12SS, Thorlabs; L4: AC508-100-A-ML, Thorlabs; DM1: T550lpxr, Chroma; DM2: T505lpxr, Chroma; Laser 2: 0488L-13A, Integrated optics. Laser 3: 0405L-13A, Integrated optics. DM3: AT440DC, Chroma; 2D Galvo Scanner: GVS002, Thorlabs; L5 and L6: AC254-100-A-M L, Thorlabs; F1: ET519/26m, Chroma; L7: ACL2520U, Thorlabs; PMT: PMT2101, Thorlabs; Obj: RMS20X, Olympus; FPGA: PXle-7841r, National Instruments. Bpod: Sanworks.

FIG. 7A is a schematic of the working principle of the in-fiber photonic crystal and a representative photo of such microstructure fabricated using laser micromachining.

FIG. 7B is a schematic of the working principle and simulation result of the multi-layer gradient filter. It is noted the combination of the principles in FIGS. 7A and 7B enables depth-dependent fluorescence excitation and collection. DM: dichroic mirror.

FIG. 8 is a schematic of the working principle of the laser-engineered fiber-optic implant and interrogation system.

FIG. 9A is a photo of a prototype laser-engineered fiber.

FIG. 9B is an image showing one position of depth-selective emission along the fiber axis (scale bar: 200 μm).

FIG. 9C is an image of another position of depth-selective emission along the fiber axis (scale bar: 200 μm).

FIG. 9D is an image of yet another position of depth-selective emission along the fiber axis (scale bar: 200 μm).

FIG. 9E is an image of one angle of angle-selective emission around the fiber (scale bar: 200 μm).

FIG. 9F is an image of another angle of angle-selective emission around the fiber (scale bar: 200 μm).

FIG. 9G is an image of yet another angle of angle-selective emission around the fiber (scale bar: 200 μm).

FIG. 9H is a schematic for light collection evaluation setup.

FIG. 91 is an image of the fiber cross-section showing the light collection at designated angular locations.

FIG. 10A is a schematic of the fiber probe.

FIG. 10B is a schematic of the workflow of the interrogation system.

FIG. 11A is a schematic showing laser micro-machining with point-by point laser inscription. A femtosecond laser beam is focused into a specific core, inducing a local modification of the optical refractive index and creating a nano-void. Scale bar: 3 μm. A grating is inscribed by translating the fiber along the axial direction while being exposed to periodic laser pulses. The bottom left inset shows a bright-field light microscope image of a laser-inscribed grating with a 650-nm pitch (i.e., the interval between two adjacent nano-voids).

FIG. 11B is a set of schematics and images illustrating the physical processes underlying the femtosecond pulse-induced localized modification of the refractive index. A representative scanning electron microscope image of the laser-inscribed nano-void is shown at the bottom left. Scale bar: 1 μm.

FIG. 12A is a schematic of the working principle of grating light emitter. A: light wavelength. A: grating pitch. ϕB: diffraction angle. L: grating length.

FIG. 12B is a set of images showing the emission profiles of a 150-μm-long grating (A=650 nm) at 450 nm, 532 nm, and 660 nm. The light beam was visualized in a mixed fluorophore solution. The top row shows side views, where the fiber boundaries are marked by dashed lines. The bottom row shows cross-sectional views, where the boundaries of the light dump are marked by dashed circles. The power emission efficiencies are 75.7% (at 450 nm), 54.7% (at 532 nm), and 29.4% (at 660 nm). Scale bar: 100 μm in both the horizontal and vertical directions.

FIG. 12C is a pair of graphs of normalized intensity profiles of the depth-confined (top) and radial-confined (bottom) illumination at 450 nm, 532 nm, and 660 nm. The full-wave-half-maximum (FWHM) values of the depth-confined illumination are 41.5 μm (at 450 nm), 35.9 μm (at 532 nm), and 29.5 μm (at 660 nm). The FWHM values of the radial-confined illumination are 16° (at 450 nm), 18° (at 532 nm), and 21° (at 660 nm).

FIG. 12D is a graph of the relationship between the first-order diffraction ratio and the tilting angle of the nano-void, measured experimentally at the three wavelengths.

FIG. 12E is a graph of the relationship between the diffraction angle and pitch of the fiber grating, measured experimentally (marks) and calculated theoretically (curves) at the three wavelengths.

FIG. 12F is a graph of the relationship between the emission efficiency and length of the fiber grating, measured experimentally (marks) and fitted numerically (curves) at the three wavelengths.

FIG. 13A is a schematic of the interrogation setup for simultaneous activation of multiple emission sites using a spatial light modulator (SLM).

FIG. 13B is a schematic of an alternative interrogation setup for single-site activation through point-to-point scanning using a galvo scanner.

FIG. 13C is a set of images of examples of reconfigurable light emission at 473 nm from the packaged PRIME fiber, including single-site, multi-site, and large-volume illumination. The top and bottom rows show the profiles in the axial and radial directions, respectively, where the insets show the corresponding input patterns. The laser emission is visualized in a fluorescein solution and pseudo-colored. Scale bar: 500 μm.

FIG. 13D is a schematic and 2 examples of dynamically reconfigurable light emission. Light is alternately emitted to the two opposite sides of a PRIME fiber, and two photodiodes (PD1 and PD2) are used to measure the power. Switching speed from 2 to 5 Hz with a contrast over 20:1 was demonstrated.

FIG. 14A is a schematic of the working principle of the system of the present disclosure.

FIG. 14B is a set of schematics and graphs representing the axial position resolvability.

FIG. 14C is a schematic and graph representing the radial direction resolvability.

FIG. 15A is an illustration of the working principle of the prime fiber.

FIG. 15B is an optical microscopic image of the cross-section of a multicore fiber. The insert shows a zoomed-in view (scale bar: 5 μm).

FIG. 15C is an illustration of a laser-inscribed grating light emitter to redirect the light propagating in the core for emission from the side of the fiber at a specific location and along a specific direction.

FIG. 15D is a is an illustration of reconfigurable light emission pattern by inputting light into different cores.

FIG. 15E is an exemplary photo of a packaged PRIME fiber with 1,200 grating light emitters spanning 5 mm along and 360° around the fiber.

FIG. 16A is a schematic of the fabrication of multiplexed fiber gratings in a multicore fiber to redirect the light for side emission at designated axial locations (z) and radial directions ($).

FIG. 16B is a set of maps showing the designation of fiber cores for illumination at different axial locations and radial directions (r0=30 μm, R=75 μm). The locations and directions are linearly encoded using the radii (r) and polar angles (0), respectively.

FIG. 16C is a set of photos showing scalable axial coverage with different a. The photos show 1 mm (a=13.3), 3 mm (a=40), and 5 mm (a=66.7) coverage, respectively. Scale bar: 1 mm.

FIG. 16D is a schematic of the interrogation setup for the PRIME fiber. A 2D hologram generated by a spatial light modulator is projected onto a multicore fiber patch cable, which is then coupled into the PRIME fiber via a standard butt-to-butt connection. The hologram pattern is relayed by the patch cable to the PRIME fiber for controlled side emission at specific axial locations and along specific radial directions. The coupling efficiency of the butt-to-butt connection is experimentally measured by using a dummy PRIME fiber with neither grating light emitters inside nor a light dump at the distal end, which is termed “dummy probe” in the insets. The structural similarity index between the input patterns of the patch cable and output patterns of the fiber probe is 0.85, indicating a decent coupling efficiency. Scale bar: 50 μm.

FIG. 17A is a picture of the optrode assembly that bundles the PRIME fiber and a two-shank NeuroNexus probe with 16 electrodes per shank. The bottom shows a zoomed-in view of the fiber and the electrodes. Scale bar: 500 μm.

FIG. 17B is a schematic of the optrode that was acutely inserted into the frontal cortex of an anesthetized Thy1-ChR2-YFP mouse for simultaneous optogenetic modulation and electrical recording.

FIG. 17C is an image of histology results illustrating the implanted location. Scale bar: 500 μm.

FIG. 17D is a set of schematics and showing spatially specific optogenetic modulation of the local field potentials (LFP) and current source density (CSD) using a single PRIME fiber.

FIG. 18A is a schematic of the targeted beams (L1-L5, and R1-R5) and the eight tetrodes (TR1-TR4 and TL1-TL4).

FIG. 18B is a set of graphs representing spike rasters and peri-stimulus time histograms of Neuron 1-3 under different illumination patterns.

FIG. 18C is a pair of graphs of quantifications of the lateral and depth specificity of the single-unit activation to light illumination. The upper shows the lateral specificity where less than 30% of the light-responsive units found on one shank responded to a contralateral stimulation (vs 95% for the ipsilateral stimulation). The bottom shows depth specificity, with the center representing the location of the light beam where the maximum firing rate of each unit is detected. The x-axis labels the light-beam moving away (up or down) to various positions. The results indicate that shifting the light beam about 200 μm up or down drops the maximum firing rate by 80% (i.e., from 100 Hz to 20 Hz).

FIG. 19A is a schematic of the open-field task setup.

FIG. 19B is an image of histological results illustrating the implanted location (Scale bar: 500 μm).

FIG. 19C is an illustration of two defensive behaviors triggered by different illumination patterns: shallow stimulation induces speed reduction, while deep and lateral stimulation induce escape and turning.

FIG. 19D is a schematic of pseudo-random light delivery patterns across trials, and single-trial movement trajectories for axial stimulations.

FIG. 19E is a set of graphs of normalized orientation over time during axial stimulations, where the radius represents time. The darker line represents the trial-average, while the lighter lines represent single-trial orientations.

FIG. 19F is a pair of graphs of time courses of averaged locomotion speed during a 2-s stimulation with different patterns. Solid lines and shaded areas indicate the mean and standard error of the mean (s.e.m.), respectively. Top right shows the mean speeds calculated during stimulation and 1 s after stimulation across patterns. The lines of the same color represent single-trial data from the same individual.

FIG. 19G is a graph of unsupervised behavior clustering results using B-SOiD. The behavior syllable with the highest occurrence frequency in each illumination pattern of the axial emission was selected for frequency quantification. The occurrence frequency of each of those representative behavior syllables was then compared for different illumination patterns.

FIG. 19H is a schematic of pseudo-random light delivery patterns across trials, and single-trial movement trajectories for radial stimulations.

FIG. 191 is a set of graphs of normalized orientation over time during radial stimulations, where the radius represents time. The darker line represents the trial-average, while the lighter lines represent single-trial orientations.

FIG. 19J is a pair of graphs of time courses of averaged locomotion speed during a 2-s stimulation with different patterns. Solid lines and shaded areas indicate the mean and standard error of the mean (s.e.m.), respectively. Top right shows the mean speeds calculated during stimulation and 1 s after stimulation across patterns. The lines of the same shade represent single-trial data from the same individual.

FIG. 19K is a graph of unsupervised behavior clustering results using B-SOiD. The behavior syllable with the highest occurrence frequency in each illumination pattern of the radial emission was selected for frequency quantification. The occurrence frequency of each of those representative behavior syllables was then compared for different illumination patterns.

FIG. 20 is a block diagram schematically illustrating a system in accordance with one aspect of the disclosure.

FIG. 21 is a block diagram schematically illustrating a computing device in accordance with one aspect of the disclosure.

FIG. 22 is a block diagram schematically illustrating a remote or user computing device in accordance with one aspect of the disclosure.

FIG. 23 is a block diagram schematically illustrating a server system in accordance with one aspect of the disclosure.

FIG. 24A is an illustration of the selective emission and collection through a single side-emission-collection fiber.

FIG. 24B is an illustration of the selective emission and collection through a single side-emission-collection an array of side-emission-collection fibers.

FIG. 24C is a flow chart of the imaging process described in Example 7.

FIG. 25A is a schematic of the setup for ultrafast laser direct writing.

FIG. 25B is a set of images showing that grating morphology is controlled by varying the shape and/or orientation of laser beam. Scale bar: 3 μm.

FIG. 26 is a schematic of the setup for characterizing the emission properties of the PRIME fiber.

FIG. 27A is a schematic of the interrogation setup for simultaneous activation of multiple emission sites using a spatial light modulator (SLM).

FIG. 27B is a schematic of an alternative interrogation setup for sequential single-site activation through point-to-point scanning using a Galvo scanner.

FIG. 28 is a schematic and set of images showing the characterization of the emission profile of a fiber grating emitter after passing through multiple layers of cores. The upper and lower rows show the side and cross-section views of the emission from a 450-nm laser, respectively. Scale bar: 200 μm.

FIG. 29A is a set of graphs of the variation in the power coupling efficiency with different numbers of cores per group. The efficiencies are 39.8+13.3%, 39.3+7.2%, and 40.6+5% for 1, 15, and 26 cores per group, respectively.

FIG. 29B is a pair of graphs of the profiles of the light emission by a 2.5-mm-long PRIME fiber along the fiber axis (the upper) and the radial direction (the below) in different trials. Variations in the emission depth and direction are+41 μm and +10°, respectively.

FIG. 30A is a schematic of the timeline of behavior testing with pseudorandom light delivery patterns across trials.

FIG. 30B is a graph of time courses of single-trial locomotion speed during 2-s stimulation with different axial patterns for individual animal A1.

FIG. 30C is a graph of time courses of single-trial locomotion speed during 2-s stimulation with different axial patterns for individual animal B2.

FIG. 30D is a graph of time courses of single-trial locomotion speed during 2-s stimulation with different radial patterns for individual animal A1.

FIG. 30E is a graph of time courses of single-trial locomotion speed during 2-s stimulation with different radial patterns for individual animal B2.

FIG. 31A is a set of graphs of single-trial movement trajectories during axial optogenetic stimulation.

FIG. 31B is a set of graphs of normalized orientation over time during axial stimulation. The radius represents time. The darker line represents the trial-average, while the lighter lines represent single-trial orientations.

FIG. 31C is a graph of time courses of averaged locomotion speed during 2-s axial stimulation. Different line shades correspond to different stimulation patterns. Solid lines and shaded areas indicate the mean and standard error of the mean (s.e.m.), respectively.

FIG. 31D is a set of graphs of single-trial movement trajectories during radial optogenetic stimulation.

FIG. 31E is a set of graphs of normalized orientation over time during radial stimulation. The radius represents time. The darker line represents the trial-average, while the lighter lines represent single-trial orientations.

FIG. 31F is a graph of time courses of averaged locomotion speed during 2-s radial stimulation. Different line shades correspond to different stimulation patterns. Solid lines and shaded areas indicate the mean and standard error of the mean (s.e.m.), respectively.

FIG. 32A is a schematic diagram illustrating a laser-direct writing portion of a batch fabrication strategy for the PRIME fiber.

FIG. 32B is a schematic diagram illustrating a cleaving portion of a batch fabrication strategy for the PRIME fiber.

FIG. 32C is a schematic diagram illustrating a light-dump coating portion of a batch fabrication strategy for the PRIME fiber.

FIG. 32D is a schematic diagram illustrating a coating curing portion of a batch fabrication strategy for the PRIME fiber.

FIG. 32E is a schematic diagram illustrating a tip machining portion of a batch fabrication strategy for the PRIME fiber.

FIG. 32F is a schematic diagram illustrating a ferrule assembly portion of a batch fabrication strategy for the PRIME fiber.

### DETAILED DESCRIPTION OF THE INVENTION

The present disclosure is based, at least in part, on the discovery that multi-core optical fibers may be modified with light emission modifications and/or light collection modifications to direct light propagating along the light-guiding cores in laterally outward or sideways direction relative to the propagation axis of the light-guiding core or receive light produced by a source positioned laterally outwards or sideways relative to the propagation axis of the light-guiding core, respectively. By patterning the spacing of the light emission and collection modifications within the multi-core optical fiber, the multicore optical fiber may be used to produce and receive patterned light in a reconfigurable manner, as described in additional detail herein.

Disclosed is a single implantable fiber-optic device that enables dynamic and reconfigurable light emission and collection in two spatial dimensions, along and around the fiber (i.e., the longitudinal axis and the polar axis, respectively). The device includes a laser-engineered optical fiber and an interrogation system. The optical fiber used for laser engineering contains multiple light-guiding cores and can be made of pristine/doped fused silica or optically transparent polymers. The diameter of the fiber ranges from tens of micrometers to a few millimeters, depending on application requirements. A short-pulsed (e.g., femtosecond or picosecond) laser beam is focused into individual light-guiding cores to modify the material's refractive index point by point. By doing so, a designed pattern of refractive index modification can be introduced to realize desired interactions with both the light propagating inside the cores and entering from the ambient environment. In turn, the light propagating inside the cores can be emitted out of the fiber, and the light from the outside can be collected into the cores. The sizes, locations, and properties (i.e., wavelength and directivity) of the light emitting and collecting sites can be precisely controlled by tailoring the laser engineering parameters, offering excellent flexibility for different applications. The morphology of the fiber tip can be flat, rounded, or tapered to accommodate different application requirements. A light-absorbing layer can be coated at the fiber tip to dump any residual light.

The interrogation system is to launch light into and to collect light from the individual cores of the laser-engineered optical fiber. The launching part can be realized by a point-by-point scanning system (e.g., a galvanometer or MEMS scanner) or a pattern projection system (e.g., a spatial light modulator (SLM) or digital mirror device (DMD)). Either incoherent or coherent light sources can be utilized. The collection part can be a CMOS or CCD camera for 2D imaging or a point detector for 1D recording, such as a photodiode and a photomultiplier tube (PMT).

Because of the high spatial resolution and superb molecular sensitivity and specificity, light-based imaging, sensing, and manipulation have been widely used in biomedicine. However, the limited tissue penetration of light, in comparison to non-optical approaches such as ultrasound and magnetic resonance, has been a major limiting factor for its practical applications, especially clinical translation.

To address this major limitation, fiber-optic techniques have been developed for minimally invasive access to deep tissues in animals and humans and have found many promising applications, including but not limited to fiber photometry, optogenetic stimulation, micro-endoscopy, sensing, and laser ablation surgery. However, most of the existing fiber-optic techniques rely on the emission and collection of light at the fiber tip, which limits the spatial coverage of a single fiber implant to a very small tissue volume near the fiber tip. To expand the spatial coverage, multiple fiber implants are usually required, which, however, inevitably increases the invasiveness and complexity of the procedures. Furthermore, due to the limited number of fibers that can be implanted in a subject, multiple subjects are often needed for comprehensive studies, which may introduce confounding factors originating from inter-subject and/or inter-procedure variations.

Recently, tapered optical fibers have been adopted to extend light emission and collection from the fiber tip to a few sparsely distributed locations along the fiber axis. However, with a single implant, this technique can only provide 1D coverage along the fiber axis and suffers from poor spatial resolution and length coverage, which significantly limits its applicability in animal research and its clinical translatability.

Disclosed herein is a first-of-a-kind laser-engineered single-fiber implant that offers dynamic and reconfigurable light emission and collection in 2D spatial dimensions, representing a significant leap over existing fiber-optic techniques. The dynamic and reconfigurable light emission and collection not only maximizes the spatial coverage of a single fiber implant, but also enables spatially resolved recording and/or manipulation over a considerable tissue volume. Fulfilling and exceeding the functionality of multiple implants with existing fiber-optic techniques, the single implantable fiber-optic device significantly reduces the required number of fiber implants and subjects, reduces invasiveness, simplifies the procedures, and reduces confounding factors. In addition, the emitting/collection properties, spatial resolutions, and length coverage are fully customizable for applications in both animals and humans.

In various aspects, a fiber-optic bi-directional interface is disclosed that comprises a modified multi-core optical fiber comprising a plurality of light-guiding cores uniformly distributed over the cross-sectional diameter of the fiber. Prior to modification, the multi-core optical fiber is designed to minimize cross-talk between individual cores so that each light-guiding core functions as an isolated bi-directional light channel that allows light to enter and exit only at the distal ends of each core. In various aspects, the multi-core optical fiber may contain any suitable number of light-guiding cores distributed within any suitable fiber diameter without limitation. Non-limiting examples of suitable multi-core optical fibers include a 160 μm-diameter multi-core fiber containing 1600 light-guiding cores, a 215 μm-diameter multi-core fiber containing 3000 light-guiding cores, and a 300 μm-diameter multi-core fiber containing 6000 light-guiding cores. In one exemplary aspect, the multi-core optical fiber is a commercially-available 160 μm-diameter multi-core glass fiber (FIGH-016-160S, Fujikura) containing 1600 light-guiding cores uniformly distributed over the fiber's cross-section.

In various aspects, at least a portion of the plurality of light-guiding cores includes a light emission modification or a light collection modification. Each light emission modification or light collection modification comprises a permanent material modification at a focal point defined within a single light-guiding core. In various aspects, the light emission modifications are configured to direct light propagating along the light-guiding cores in a laterally outward or sideways direction relative to the propagation axis of the light-guiding core. In various other aspects, the light collection modifications are configured to receive light produced by a source positioned laterally outwards or sideways relative to the propagation axis of the light-guiding core.

In various aspects, the light emission modifications and light collection modifications are distributed along an emission/collection span extending a predetermined distance along the propagation axis of the light-guiding cores. In various aspects, the light emission and collection modifications are distributed lengthwise such that an unimpeded radial or lateral light path is defined through surrounding light-guiding cores so that light may be emitted or collected by each light emission and collection modification, respectively, unimpeded by other modifications within other light-guiding cores.

In various aspects, the emission/collection span may be any suitable length along the axis of the light-guiding cores without limitation. In some aspects, the emission/collection span may be at least 1 mm, at least 2 mm, at least 3 mm, at least 4 mm, at least 5 mm, or longer. In other aspects, the emission/collection span may be less than 1 mm, less than 2 mm, less than 3 mm, less than 4 mm, less than 5 mm, or less than 10 mm.

In various aspects, the spacing of the light emission and collection modifications along the light-guiding cores of the fiber-optic bi-directional interface may be configured to perform optically based methods at a depth resolution of about 200 μm, about 150 μm, about 100 μm, about 50 μm, or higher resolution.

Any suitable fabrication method may be used to form each material modification within each light-guiding core without limitation. In some aspects, a hybrid laser micromachining method is used to form the material modifications.

In various aspects, the light emission modifications may be any suitable modification capable of directing light propagating along a light-guiding core in an outward radial or lateral direction relative to the core axis. Non-limiting examples of suitable light emission modifications include photonic crystals and fiber Bragg gratings (FNGs). In various aspects, the light collection modifications may be any suitable modification capable of collecting light propagating along a light-guiding core in an inward radial or lateral direction relative to the core axis. Non-limiting examples of suitable light collection modifications include total internal reflection (TIR) mirrors and multi-layer gradient filters.

By choosing the location of the laser modifications, the location of the emission and collection site can be encoded by different cores. More light collection modifications such as TIR mirrors may promote collection efficiency but reduce the available number of light emission channels, such as FNGs, due to the total number of available light-guiding cores. In some aspects, the allocation of the emission and collection structures may be selected to enhance the resolution and collection efficiency.

In various aspects, the distal tip of the multi-core optical fiber is coated with a light-absorbing material, such as an epoxy-carbon mixture to absorb the un-emitted light. Without being limited to any particular theory, the thickness of the light-absorbing layer may be selected to minimize the thermal effect. In various aspects, the multi-core fiber-optic probe is packaged in a form that is compatible with existing systems such as standard fiber optogenetics and photometry systems to ensure seamless usage.

Without being limited to any particular theory, the distribution of material modifications within a plurality of light-guiding cores of the multi-core optical fiber provides for the delivery and collection of light in a variety of different formats. By launching light into different combinations of cores, depth-selective, patterned, or large-volume illumination can be realized within the same multi-core fiber-optic probe without changing its physical configuration.

In various additional aspects, the multi-core fiber-optic probe may be incorporated into a system configured to conduct one or more light-based procedures including, but not limited to, optogenetic stimulation, photometry, and any combination thereof. In one aspect, the system includes an optogenetic component that includes at least one light source optically coupled to the probe and configured to deliver light into the proximal end of the probe in a predetermined pattern and a light detector optically coupled to the probe and configured to receive light exiting the probe at the proximal end.

In one aspect, the system can include an interrogation system. In another aspect, the interrogation system can have an optogenetic component, a photometry component, and a control and synchronization component. In some aspects, the system can have a control element including a computing device that can include at least one processor, including but not limited to an FPGA board. The FPGA may be configured to operate the devices of the optogenetic stimulation and photometry recording elements. Lasers can be employed in a coordinated manner and an SLM to produce a predetermined pattern. The FPGA may also be operatively coupled to additional devices including, but not limited to, a behavioral experiment control unit configured to synchronize the components of the system to follow the experimental design.

In one aspect, the system can perform optogenetic excitation. In an exemplary embodiment, a 561 nm yellow-green CW laser with pulsed external modulation can be used to excite the VChR1 (peak excitation: 570 nm). The collimated laser beam can be expanded and shined onto a spatial light modulator (SLM) to generate a 2D hologram pattern. After filtering out the non-modulated light with a pinhole, the hologram pattern can be projected onto the cross-section of a home-built lead-in patch cable through an objective lens. The lightweight and flexible patch cable can be made of a pristine multi-core fiber that relays the pattern from the interrogation system to the input end of the fiber implant. The computer-generated pattern couples the light into the targeted cores and in turn excite neurons at desired locations.

In another aspect, the system can perform photometry readings. In one aspect, the system can have at least two light sources coupled to a probe. In an exemplary embodiment, two lasers with wavelengths of 405 nm (purple) and 488 nm (blue) can be used to excite the calcium-insensitive and sensitive points of the GCaMP sensors, respectively. Exciting at the isosbestic point (405 nm) is to compensate for any background fluorescence fluctuation induced by mouse motion. The two lasers can be combined via a dichroic mirror (OM3) and enter the 2D-galvo scanner for beam steering. The steered beam can be shined on the fiber patch cord through the same objective lens and relayed to the fiber implant. The laser beam can scan the light-emitting cores one by one, and at each position, the two colors alternate with out-of-phase modulation. The excited fluorescence can be coupled into the light-collecting cores and detected by a photomultiplier tube (PMT) after spectral filtering by dichroic mirrors (OM1 &2) and a bandpass filter (F1). Repeating this process generates the calcium activity mapping along the fiber track.

As described in the examples below, laser 3D microfabrication technology was used to engineer a fiber-optic interface with reconfigurable light emission capability both longitudinally and radially, allowing for depth and angular-resolved stimulation. In various aspects, the fiber-optic interface includes a single 160-μm diameter fiber-optic implant featuring 1200 independently addressable emitters spread over a 2.5 mm span and encompassing 360 degrees around the fiber. In some aspects, each emitter covers an area of 150×20 μm2, with a total coverage of 2500×500 μm2 per fiber. In some aspects, the device is packaged with a standard fiber ferrule and is compatible with butt-to-butt connectorization, thus adhering to common fiber optogenetics practices in various research areas including, but not limited to, neuroscience labs.

As further described in the examples below, the efficacy of the disclosed fiber-optic interface in one aspect was demonstrated by integrating the fiber with a silicon electrode array and used to record light-evoked neural activity across mouse cortical layers in vivo. Localized, light-evoked spike activity was detected with a longitudinal resolution of 200 μm that was selectively confined to one or the other side of the fiber when stimulating in different directions. To evaluate the device in behaving mice, the device was implanted into the superior colliculus of mice and used to deploy spatially varying light patterns. Delivering light to different layers and in different radial directions through the same fiber produced divergent behavioral responses. The results obtained in the examples below demonstrate the potential of the disclosed laser-engineered fiber-optic interface as a versatile tool for spatially reconfigurable neural stimulations in freely-behaving animals through single fibers.

### Computing Systems and Devices

In various aspects, the disclosed fiber optic optogenetic and photometry methods may be implemented using a computing system or computing device. FIG. 20 depicts a simplified block diagram of the system for implementing the computer-aided method described herein. As illustrated in FIG. 20, the computing device 300 may be configured to implement at least a portion of the tasks associated with the disclosed methods described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 and a fiber optic system 334 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

In other aspects, the computing device 302 is configured to perform a plurality of tasks associated with the disclosed computer-aided methods of performing optogenetic and photometry to characterize and modulate behavior. In some aspects, the computing device 302, user computing device 330, and/or fiber optic system 334 may be operatively connected via a network 350. FIG. 21 depicts a component configuration 400 of computing device 402, which includes database 410 along with other related computing components. In some aspects, computing device 402 is similar to computing device 302 (shown in FIG. 20). A user 404 may access components of computing device 402. In some aspects, database 410 is similar to database 308 (shown in FIG. 20).

In one aspect, database 410 includes optical data 412, algorithm data 418, and behavior data 420. Optical data 412 may include data used to operate a fiber optic system using the acquisition methods as disclosed herein. Non-limiting examples of optical data 412 include various measurements of optical signals and any parameters used to control the operation of a fiber optic device for the optogenetic and photometry methods as disclosed herein. Algorithm data 418 may include any parameters defining equations or other algorithms used to implement the behavior characterization and modulation as disclosed herein. Behavior data 420 may include any analysis or intervention signaling that characterizes and modulates behavior with the fiber optic system 334.

Computing device 402 also includes a number of components that perform specific tasks. In the exemplary aspect, computing device 402 includes a data storage device 430, an imaging component 440, an analysis component 450, and a communication component 460. The optogenetic component 440 is configured to implement optogenetic methods as described herein. The photometry component 450 is configured to implement the photometry methods as disclosed herein. The data storage device 430 is configured to store data received or generated by computing device 402, such as any of the data stored in database 410 or any outputs of processes implemented by any component of computing device 402.

The communication component 460 is configured to enable communications between computing device 402 and other devices (e.g. user computing device 330 shown in FIG. 20) over a network, such as a network 350 (shown in FIG. 20), or a plurality of network connections using predefined network protocols such as TCP/IP (Transmission Control Protocol/Internet Protocol).

FIG. 22 depicts a configuration of a remote or user computing device 502, such as user computing device 330 (shown in FIG. 20). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

FIG. 23 illustrates an example configuration of a server system 602. Server system 602 may include, but is not limited to, database server 306 and computing device 302 (both shown in FIG. 20). In some aspects, server system 602 is similar to server system 304 (shown in FIG. 20). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 20) or another server system 602. For example, communication interface 615 may receive requests from a user computing device 330 via a network 350 (shown in FIG. 20).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 22) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods.

The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics and animal behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regressions, random forest classifiers, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context.

The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Glioblastomas Alter the Spectral Content of Spontaneous Whole-Brain Activity

To demonstrate the efficacy of the fiber-optic bi-directional interface disclosed herein, the following experiments were conducted. The device was implanted in the superior colliculus of mice and used to deploy spatially varying light patterns. Light-evoked neural activities across mouse cortical layers in vivo were recorded. Delivering light to different layers and in different radial directions through the same fiber produced divergent behavioral responses.

### Example 2—Fiber-Optic Neural Interface for Bi-Directional Interrogation (Optogenetic Stimulation and Photometric Recording) in Freely Behaving Animals

In Example 2, a fiber-optic neural interface for bi-directional interrogation (i.e. optogenetic stimulation and photometric recording) in freely behaving animals, which provides an unprecedented depth-sectioning ability (50 μm) while preserving a large spatial coverage (5 mm for a single fiber), is developed. Building upon our latest progress in laser-engineered optical fibers, this neural interface can process thousands of two-way optical channels along a single fiber. This technology can be used to uncover the roles of mouse Superior Colliculus (SC) layers in distinct behaviors. The midbrain SC plays a central role in the sensorimotor transformation and controls diverse innate behaviors in all vertebrates, including rodents. Despite decades of research, a detailed functional mapping of mouse SC is still missing due to its complex structure, a densely packed three-dimensional (3D) functional organization where individual layers constitute different neural circuits and participate in distinct types of behaviors. Applying the technology to mouse SC not only showcases the fiber interface's unique capabilities but also advances understanding of the causal relationships between the SC neurons and associated behaviors.

Example 2 describes a fiber-optic neural interface that combines optogenetic stimulation and photometric recording for bi-directional interrogation of the freely behaving animal brain with sub-layer resolution and full-depth coverage. Successful development of the bi-directional fiber interface offers an enabling tool to study the relation between neurons and behaviors across regions and layers. Moreover, this technique can be utilized in mice to study the involvement of SC layers in innate dimorphic defensive behaviors. The finding offers insights into the decision-making process upon facing threats.

### Background and Significance

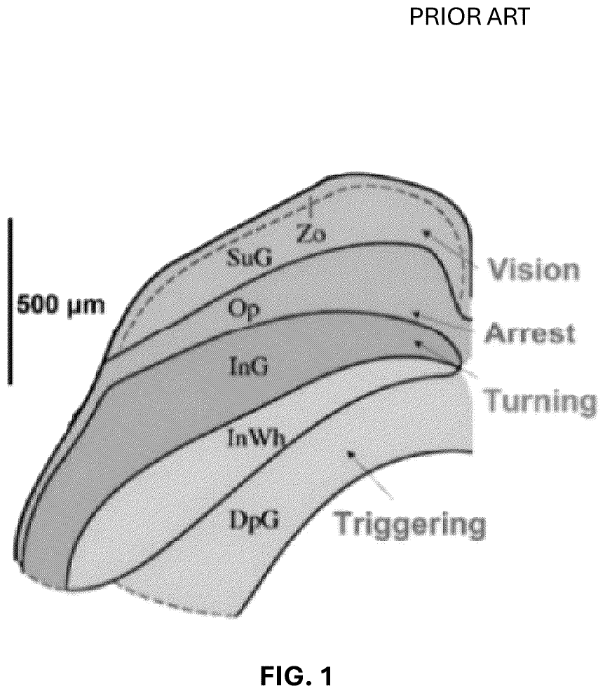

Recent advances in implantable neural interfaces offer new opportunities to perform large-scale stimulation and physiological recording in freely moving animals, facilitating our understanding of how widespread neurons guide behaviors. High-density electrodes (e.g., Neuropixels) have demonstrated the simultaneous recording of hundreds of sites across single or multiple brain regions. Nevertheless, their insufficient cell-type specificity hinders the further refinement of neuronal function, as it is known that different types of cells participate in different circuits. On the contrary, capitalizing on optogenetics and genetically encoded calcium indicators (GECI), light-based neurotechnologies have enabled large-scale, cell-type-specific neuronal interrogation. For instance, cellular-size light emitters/receivers (e.g., micro-LEDs, photodiodes, waveguides) can be integrated into a single implantable shank to interact with individual neurons in situ. However, the low electrical-to-optical conversion efficiency limits the light-emitting power and raises the concern of excessive heat when being deployed over a large scale. Besides, the reduced surface area significantly limits the receiver's sensitivity, and the in-vivo environment introduces extra thermal noise, which together compromises the signal-to-noise ratio and the reliability of the recording. On the other hand, implantable passive optical elements serve as a simple light relay for external light sources and high-sensitivity detectors, offering much higher light efficiency and recording quality. GRIN lenses have been widely used in miniaturized light microscopes (e.g., Miniscope), offering single-neuron interrogation at the expense of limited field-of-view. In contrast, optical fibers (e.g., fiber optogenetics and photometry) have been applied to interact with neurons at the population level across brain regions. Despite the scale advantage, conventional optical fibers can only reach a single layer at a time, and only a few fibers can be implanted in the same region to avoid impairing brain functions by excessive tissue damage. While recently developed tapered fibers can reach a few sites along the implantation track, their resolution is inadequate for fine layers (FIG. 1) due to their limited light manipulation capability inside the fiber. Despite tireless efforts, a versatile optical neural interface capable of high spatial resolution and extensive coverage is still lacking, yet crucial for the understanding of regional brain functions, such as those of superior colliculus (SC).

The SC, at the roof of the mid brain, is an evolutionarily conserved structure with strong functional commonalities across mammals, including rodents. In mice, SC receives projections from ˜90% of the retinal ganglion cells (RGCs) and sends projections to downstream motor regions, playing a central role in visual information processing and rapid sensorimotor transformation. In addition, the mouse SC is highly interconnected with many other brain regions, including the cerebellum, thalamus, and neocortex, which allows it to coordinate several “higher-level” functions, including attention and decision making. Together with the recent advances in mouse molecular genetic technique, physiological recording, and SC-dependent behavioral assays, mouse SC has become an emerging model for understanding the relationship between circuitry and behavior. The mouse SC is organized into several synaptic layers spanning from 1.5 to 3 mm in depth, with the thinnest portion being less than 200 μm (FIG. 1). Growing evidence supports that each layer performs a particular computation while the functional consequence depends on the inputs and outputs at particular medial and/or lateral locations within the layer, resembling the columnar microcircuitry in the cerebral cortex. Yet. how these layers compute and interact to select a final action output remains unclear. For example, anatomical tracing data suggest that the SuG and Op layers are involved in two pathways that underly the action section between freezing and escaping, but activation of the entire intermediate layers does not show a significant correlation to either behavior. The frustration of unraveling the SC functionality further emphasizes the need for a technological breakthrough to achieve layer-specific neuronal activation and full-column recording in freely behaving mice.

Despite recent advancements in implantable neural interface, they do not provide layer-specific interaction across multiple layers and regions, impeding the further understanding of how orchestrated neurons relate to behaviors. Mouse SC has been widely hypothesized to be a densely packed three-dimensional functional organization, yet the roles of each layer behind simple behaviors are still unclear.

### Results

The bi-directional fiber-optic neural interface is innovative because it enables simultaneous neuronal stimulation and recording in freely moving mice with cell-type specificity, high depth resolution. and multi-region coverage. This unique technological advance opens a new horizon for a more detailed understanding of circuit-forming mechanisms. Enabled by the proposed fiber-optic neural interface, unpacking the roles of SC in the divergent circuit underlying the two innate defensive behaviors is unprecedented.

### Reconfigurable Fiber-Optic Light Emitter.

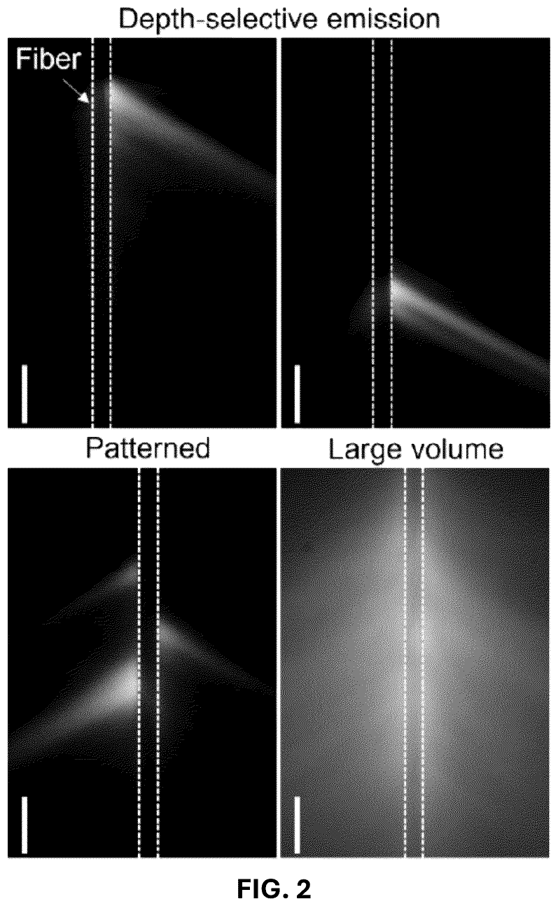

The fiber emitter consists of a single 160 μm-diameter multi-core fiber with 1,600 evenly distributed light-guiding cores. A focused laser beam was used to write fiber Bragg gratings (FBGs) into the cores. For a single core, an FBG allows the light to escape at a pre-defined location along the fiber with a spatially confined emission profile. Thus, by launching light into different combinations of cores, depth-selective, patterned, or large-volume illumination can be realized within the same fiber probe without changing its physical configuration (FIG. 2). The preliminary result demonstrates the promise of flexible light manipulation with a single fiber.

### Layer-Specific Optogenetic Stimulation in Freely Moving Mice

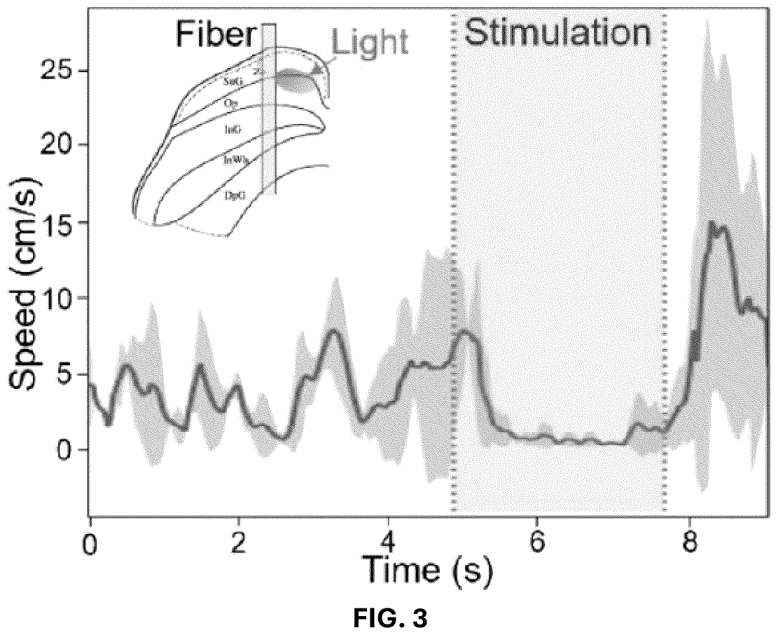

The fiber emitters were implanted into the SC region of female Thy1-ChR2-YFP mice (AP:−3.72 mm, ML: 0.6 mm). Light pulses (473 nm, 3 ms, 20 Hz, 3 seconds) were delivered during the mice's freely moving phase and freezing behavior (i.e., a decrease of locomotion speed) was observed when the intermediate layer (DV: 1.2 mm) was selectively activated (FIG. 3), consistent with previous reports. This preliminary result supports the feasibility of deploying the proposed bi-directional fiber-optic interface into freely moving animals to infer neural correlates of behaviors.

### Divergent Pathways for Dimorphic Defensive Behavior

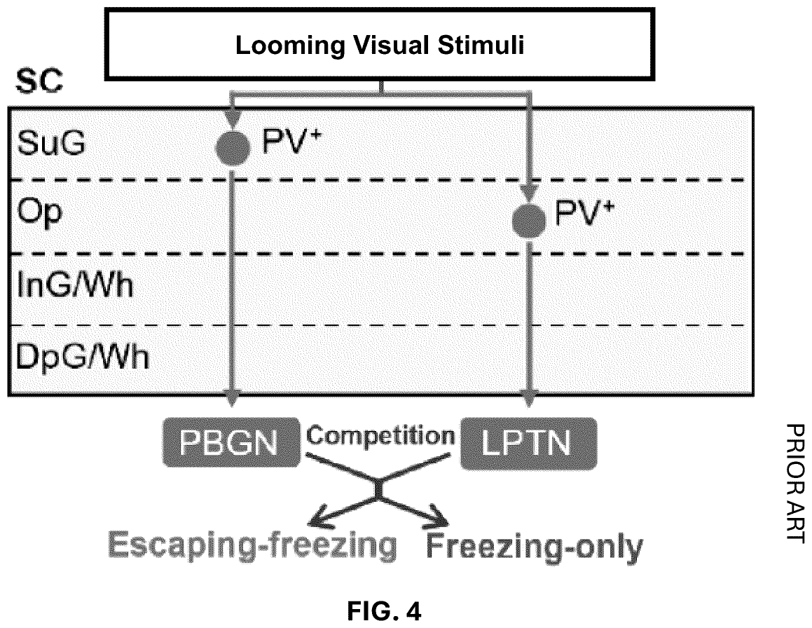

Mice under looming visual threats show two innate defensive behaviors, escape-freezing and freezing-only. It has been reported that parvalbumin-positive (PV+) neurons in SC mediate the dimorphic behavioral pattern by sending separate projections to parabigeminal nucleus (PBGN) and lateral posterior thalamic nucleus (LPTN). Based on the anterograde tracing data, two competing pathways (FIG. 4) have been proposed involving two connecting SC layers (SuG and Op). However, the current study does not reveal a significant correlation between behavioral outputs and neural activity in PBGN and LPTN, suggesting the hypothesis that the two SC layers contribute to the behavioral selection.

### Bi-Directional Fiber-Optic Probe

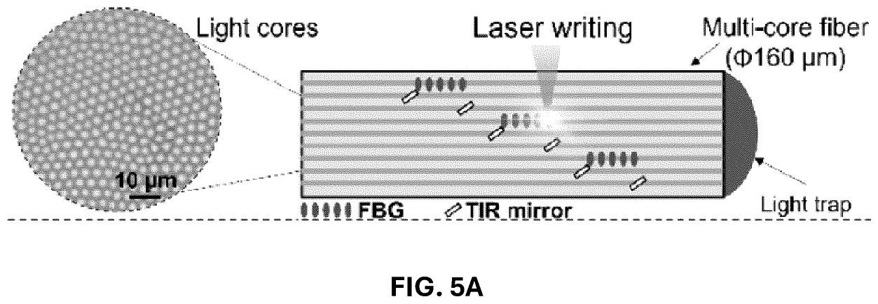

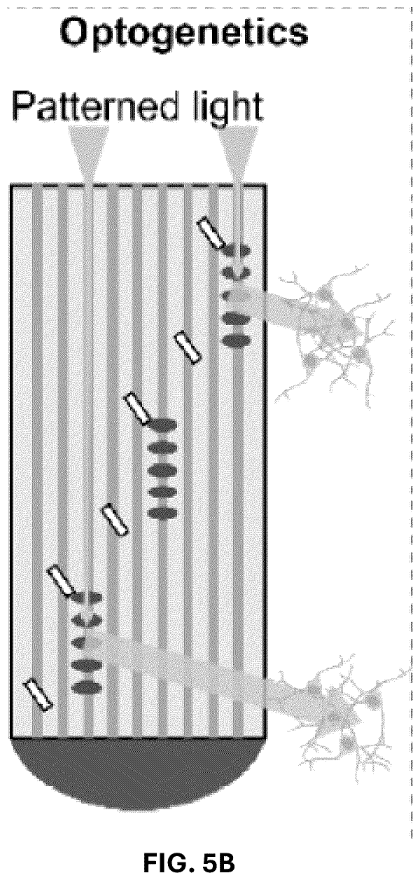

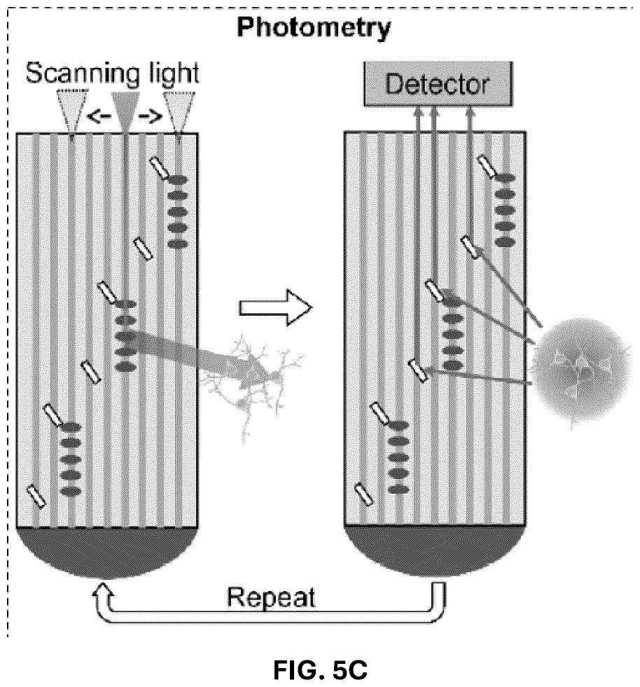

The fiber probe consists of a commercial 160 μm-diameter multi-core glass fiber (FIGH-016-160S, Fujikura), where 1600 light-guiding cores are uniformly distributed over the cross sections (FIG. 5). The fiber was specially designed so that the inter-core crosstalk is minimum, and thus each light-guiding core functions as an isolated bi-directional light channel. The pristine fiber only allows light to enter and exit at the two distal ends. To enable light to be transported from the side, laser micromachining is used to alter the light-guiding properties in individual cores. In short, owing to the transparency of glass, a laser beam can be focused into a particular core from the side at a specific location along the fiber, and a permanent material modification is introduced only within the focal spot (i.e., the targeted core) to modulate the light. A hybrid laser micromachining strategy for light emission and collection is adopted to overcome the tradeoff between efficiency and resolution in the current prototype.

### Light Emission

An FBG, a periodical point modification, is inscribed inside a specific core. Each point weakly scatters the propagating light out of the core. Owing to the periodical arrangement and wave nature of light, only the light emitting towards a specific direction will be significantly enhanced by constructive interference. As a result, a spatially confined beam (i.e., NA<0.01) can be generated whose thickness is determined by the length of the laser-modified points.

### Light Collection

Total internal reflection (TIR) mirrors are fabricated inside the fiber cores to maximize the collection efficiency. The mirror is fabricated via the laser-assisted chemical etching process, which can introduce hollow rectangular cavities inside the glass. Any light reaching the air/glass interface with an incident angle larger than the critical angle (˜45 degrees) and smaller than the fiber collection angle (˜69 degrees) is fully collected by the fiber. In other words, a single TIR mirror structure has a NA˜0.2. In addition, multiple TIR mirrors are used to collect the fluorescent emission. Together, at least two-order-of-magnitude higher collection efficiency than the current prototype can be achieved. Furthermore, TIR mirrors exhibit weak chromatic dispersion, making them ideal for multi-color photometry. By choosing the location of the laser modification, the location of the emission and collection site can be encoded by different cores. More TIR mirrors promotes collection efficiency but reduces emission channels due to the conservation of the total core numbers. Thus, the allocation of the emission and collection structures can be evaluated to optimize the resolution and collection efficiency. At last, an epoxy-carbon mixture layer is coated onto the fiber tip to absorb the un-emitted light. The layer thickness can be optimized numerically to minimize the thermal effect. The fiber probe can be packaged in the same form as standard fiber optogenetics and photometry to ensure seamless usage.

### Interrogation System.

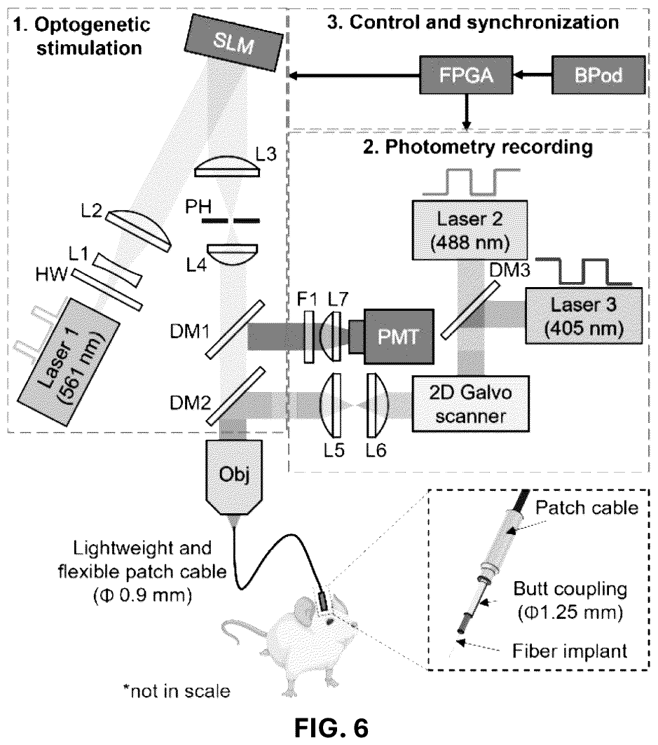

As shown in FIG. 6, the interrogation system for the bi-directional fiber probe contains 3 parts.

### Optogenetic Excitation

A 561 nm yellow-green CW laser with pulsed external modulation is used to excite the VChR1 (peak excitation: 570 nm). The collimated laser beam is expanded and shined onto a spatial light modulator (SLM) to generate a 20 hologram pattern. After filtering out the non-modulated light with a pinhole, the hologram pattern is projected onto the cross-section of a home-built lead-in patch cable through an objective lens. The lightweight and flexible patch cable is made of a pristine multi-core fiber that relays the pattern from the interrogation system to the input end of the fiber implant. The computer-generated pattern couples the light into the targeted cores and in turn excite neurons at desired locations.

### Photometry Recording

Two lasers with wavelengths of 405 nm (purple) and 488 nm (blue) are used to excite the calcium insensitive and sensitive points of the GCaMP sensors, respectively. Exciting at the isosbestic point (405 nm) is to compensate for any background fluorescence fluctuation induced by mouse motion. The two lasers are combined via a dichroic mirror (OM3) and enter the 2D-galvo scanner for beam steering. The steered beam is shined on the fiber patch cord through the same objective lens and relayed to the fiber implant. The laser beam scans the light-emitting cores one by one, and at each position, the two colors alternate with out-of-phase modulation. The excited fluorescence is coupled into the light-collecting cores and detected by a photomultiplier tube (PMT) after spectral filtering by dichroic mirrors (DM1&2) and a bandpass filter (F1). Repeating this process generates the calcium activity mapping along the fiber track.

### Control and Synchronization.

The system is controlled by a Field-Programmable-Gate-Arrays (FPGA) board and synchronized with the behavioral experiment control unit (BPod).

In another embodiment a thicker multi-core fiber with a higher core number (i.e., 3000 cores for 215 μm fiber and 6000 cores for 300 μm fiber) can be employed. The size of the fiber can be tuned to optimize the performance.

### Example 3—High Temporospatial-Resolution Depth-Resolved Fiber Photometry

Genetically encoded fluorescence indicators have enabled large-scale recording of the orchestrated neural activity in vivo. Further, recent advances in light-based neurotechnologies have extended the utility freely moving animals, facilitating understanding of the natural behavior and how it goes awry in neurological or psychiatric diseases. Specifically, two main categories of optical technologies have been developed to peer into neural activity in the freely behaving brain at very different scales. On the one hand, miniaturized light microscopy (e.g., miniscope) enables imaging of neural activity at the cellular level within a very limited field of view (FOV). One the other hand, fiber photometry enables assessment of neural activity at the population level across different depths or over multiple functional regions. While the former has the resolution power to reveal orchestrated activity among individual neurons, the latter has the scale advantage to study the complex interaction of large neuronal populations across brain regions.

The lack of resolution with the current fiber photometry is due to a simplified coupling mechanism between the brain and the fiber interface. The use of flat-cleaved fibers in conventional fiber photometry does not provide resolution for imaging but rather collects a cumulative signal from all neurons. To overcome this limitation, tapered fibers have been exploited to enable multisite fluorescence excitation and collection. However, its depth-sectioning ability is rather limited (2 or 3 sites over a mm) due to the insufficient separation of different propagating modes in the tapered fiber. To this end, a fiber photometry technique, which offers high depth-sectioning ability (50-100 μm) while preserving the large spatial coverage of conventional fiber photometry, has been developed.

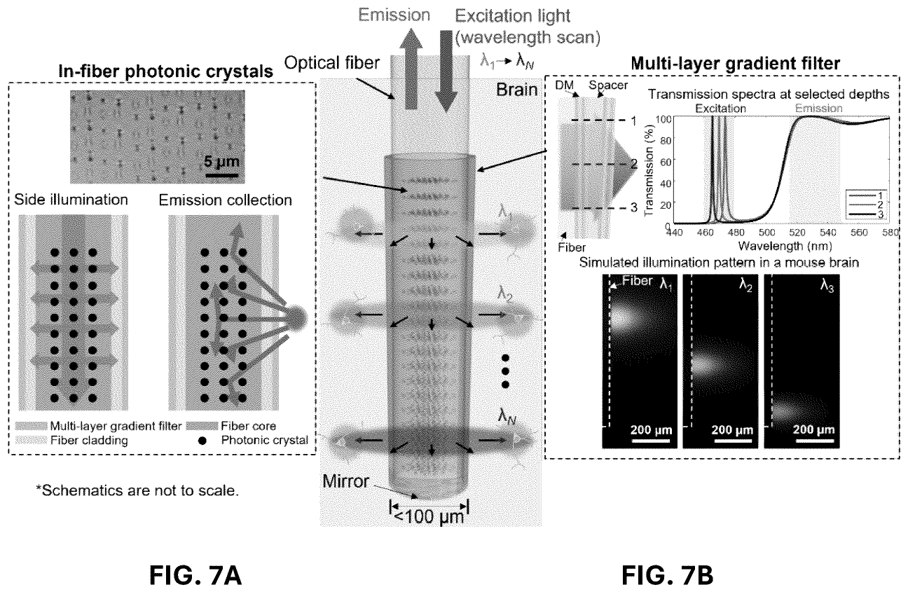

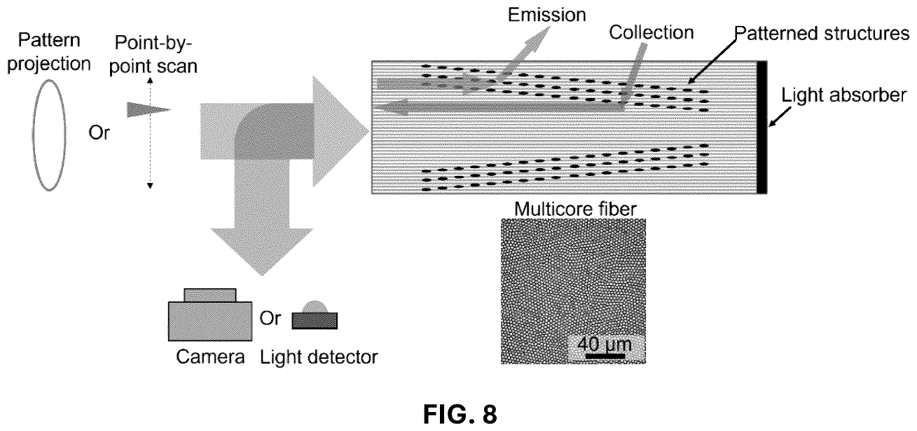

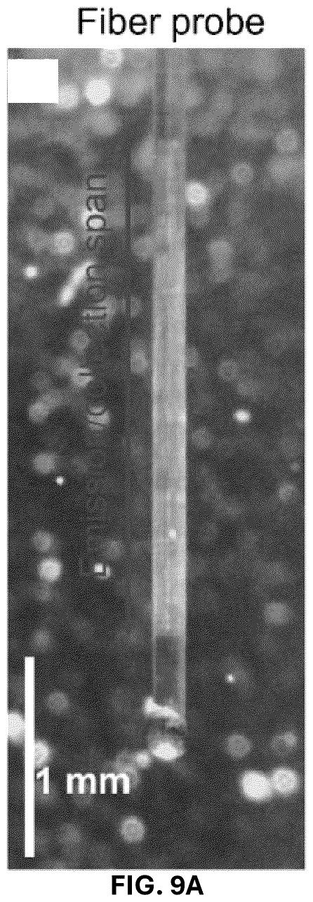

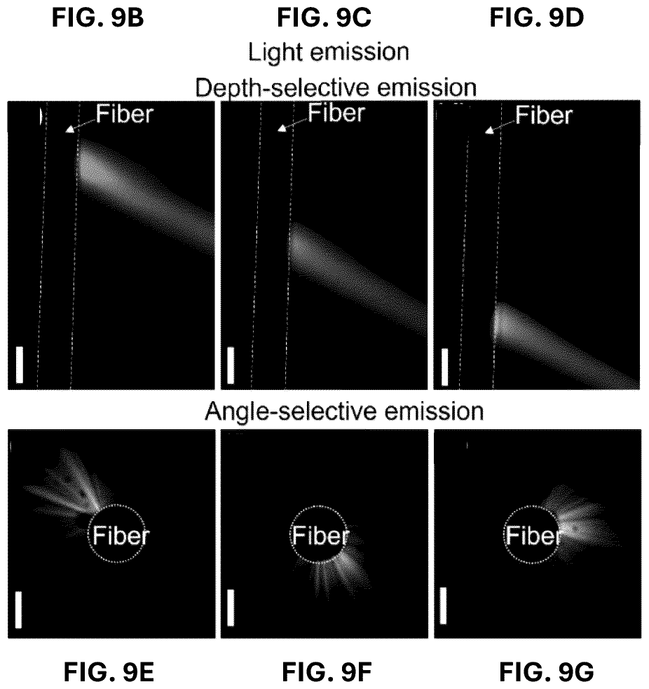

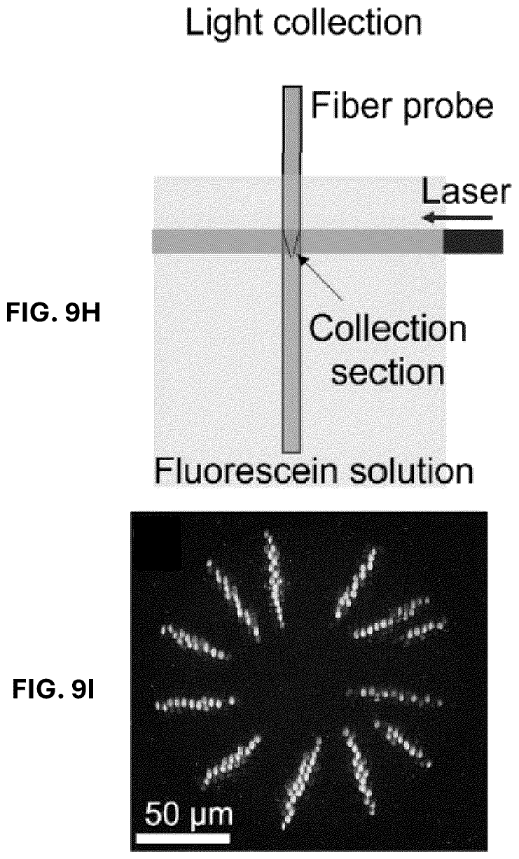

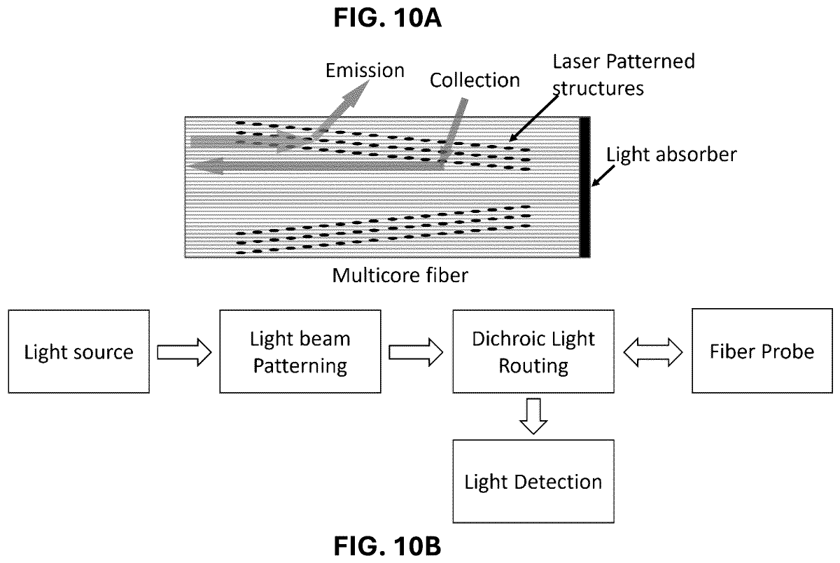

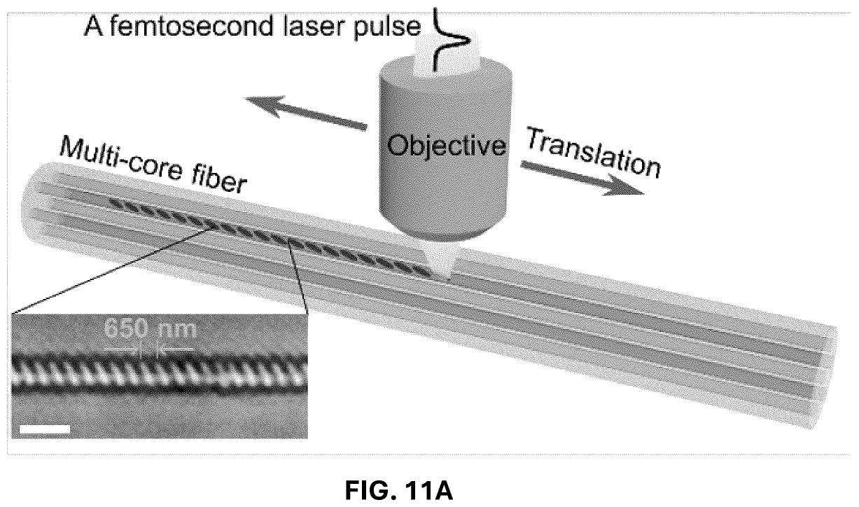

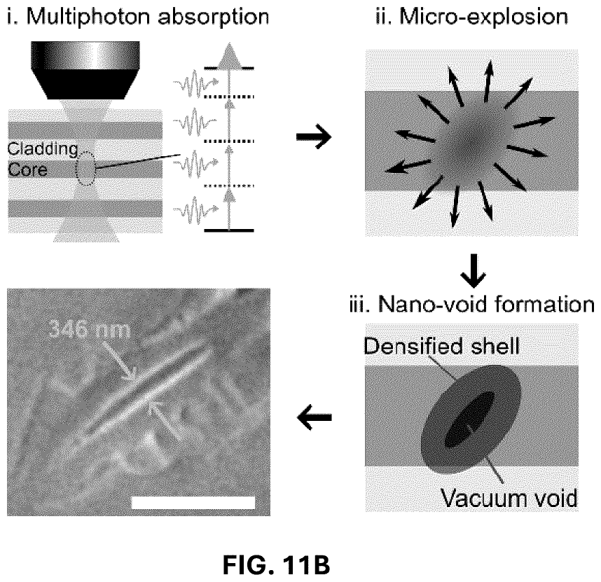

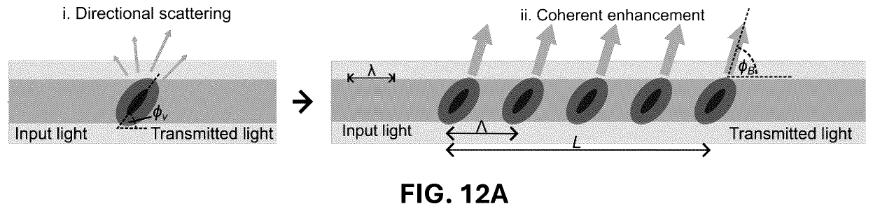

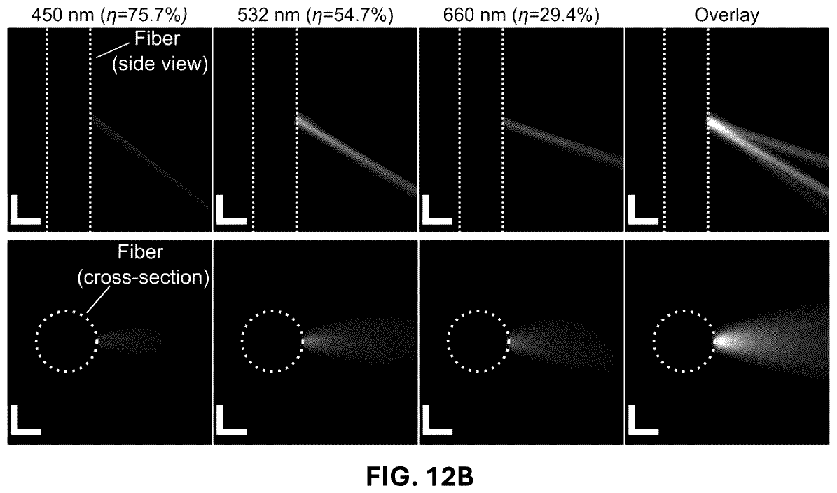

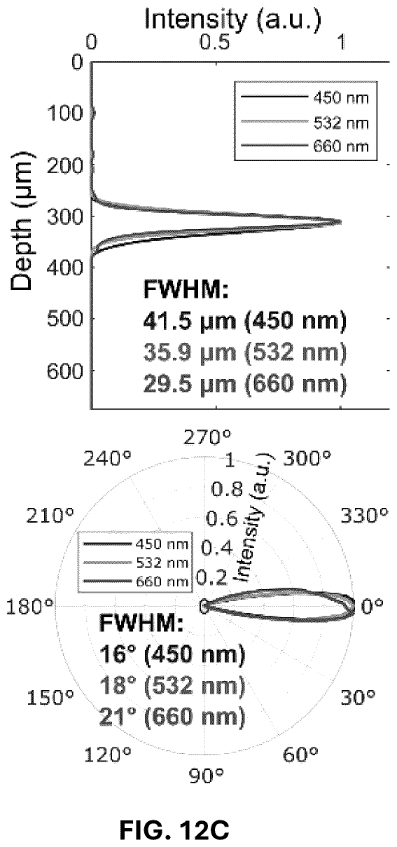

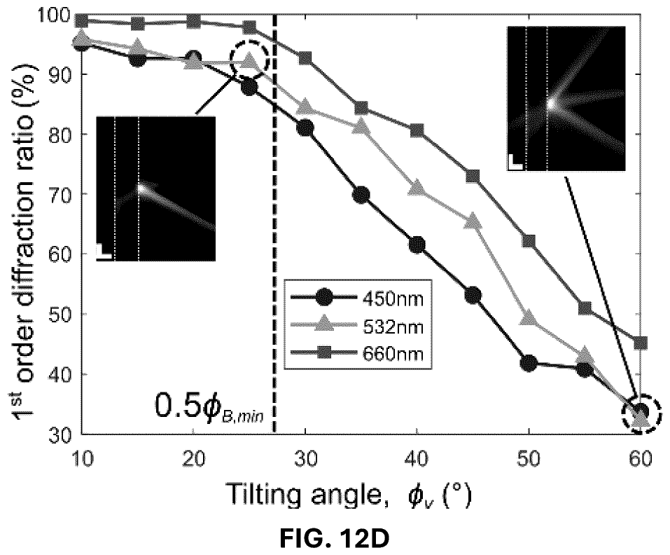

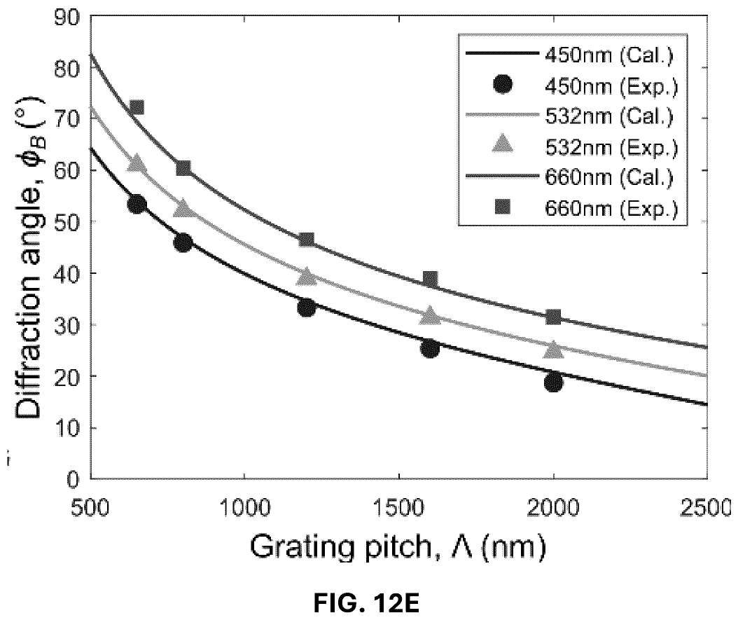

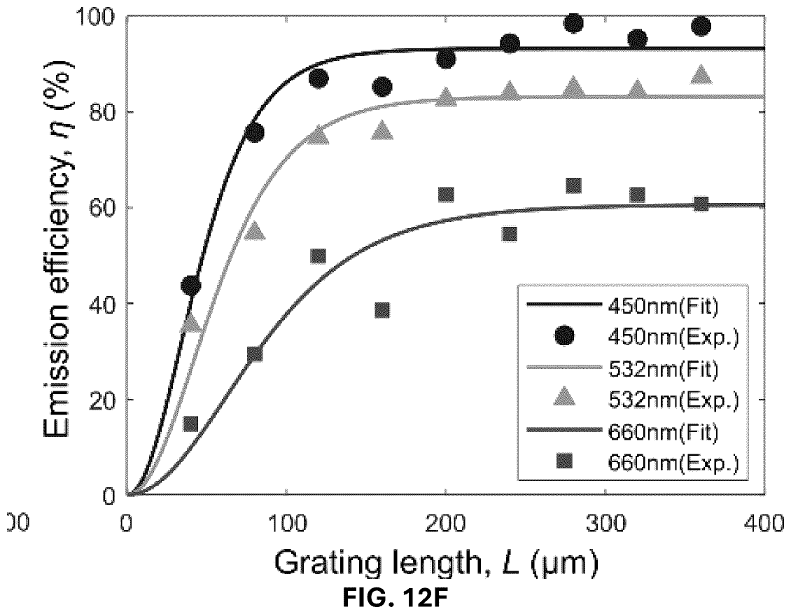

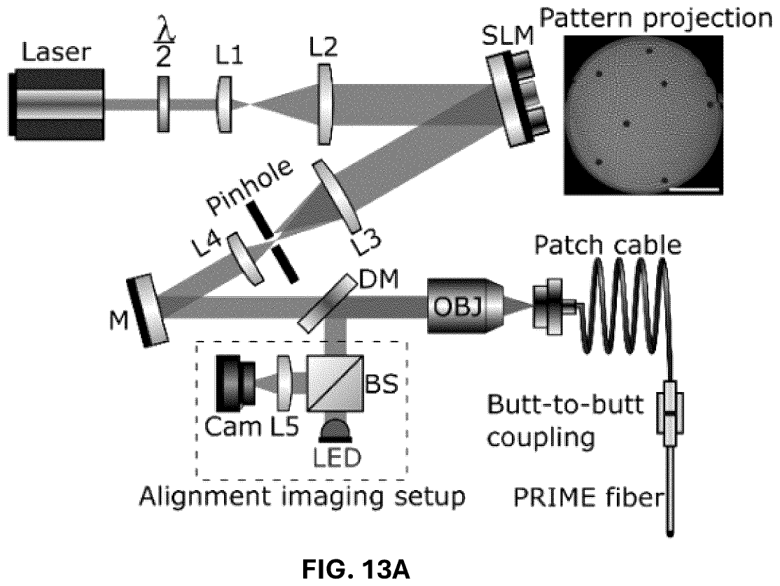

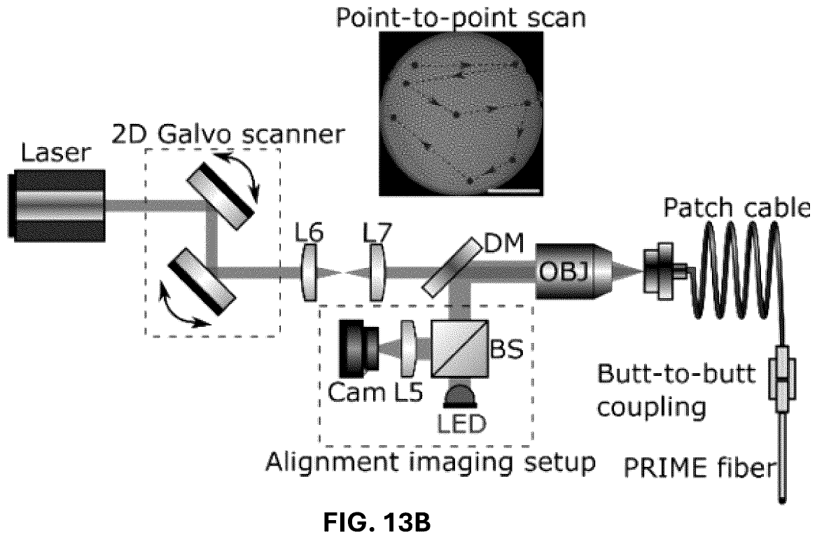

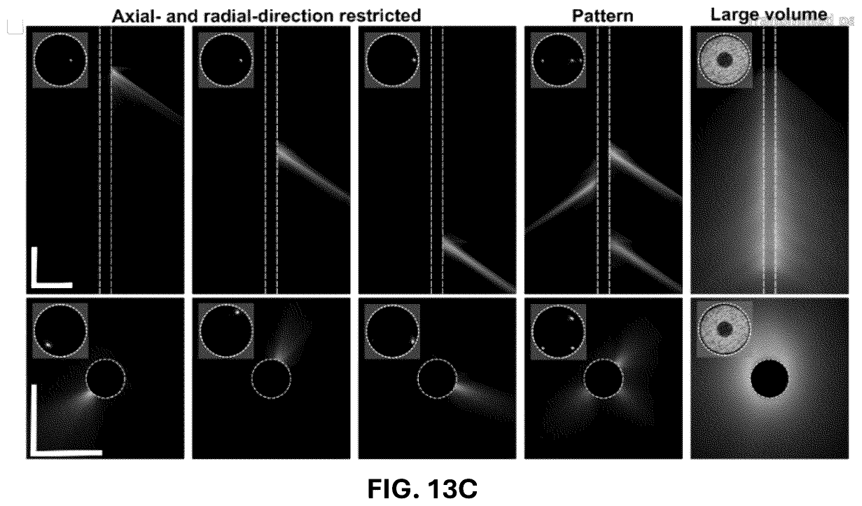

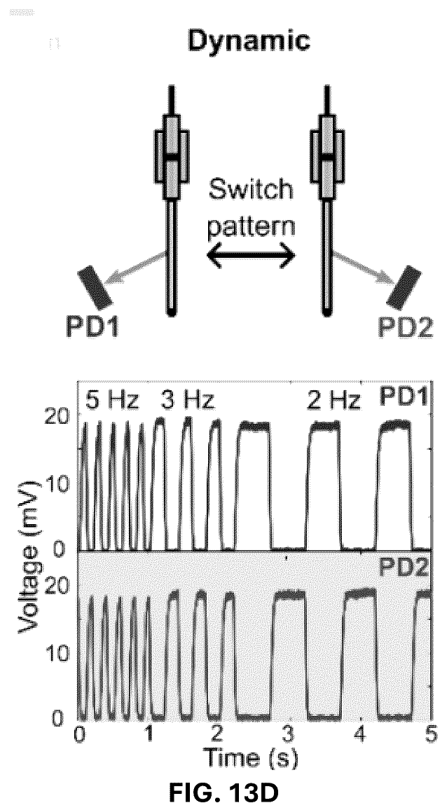

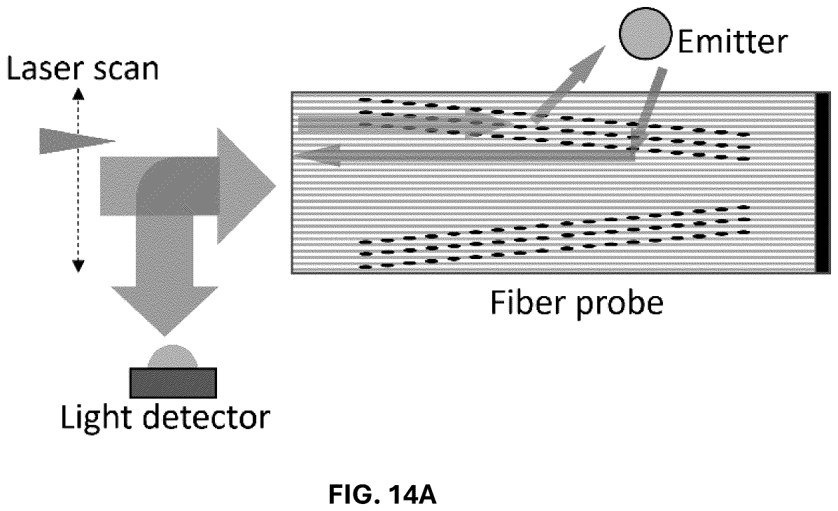

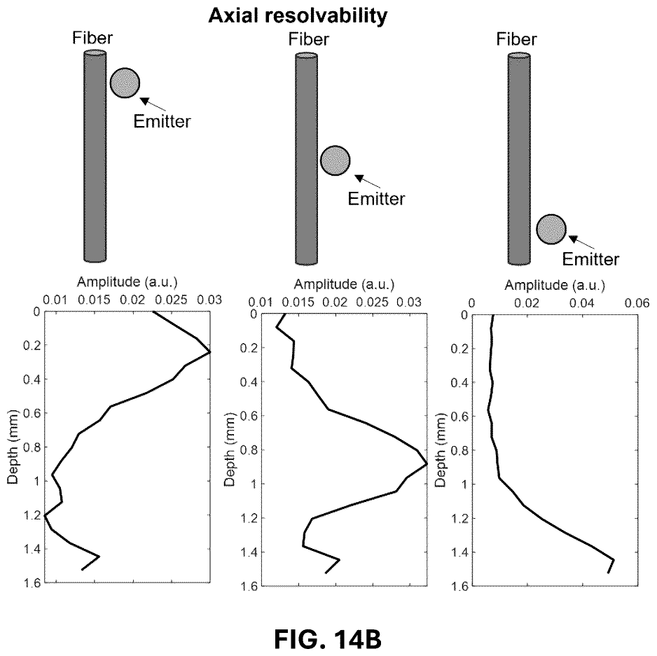

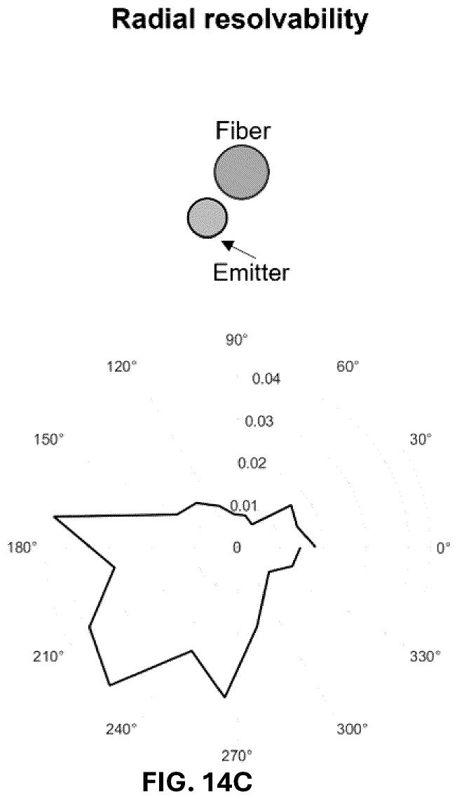

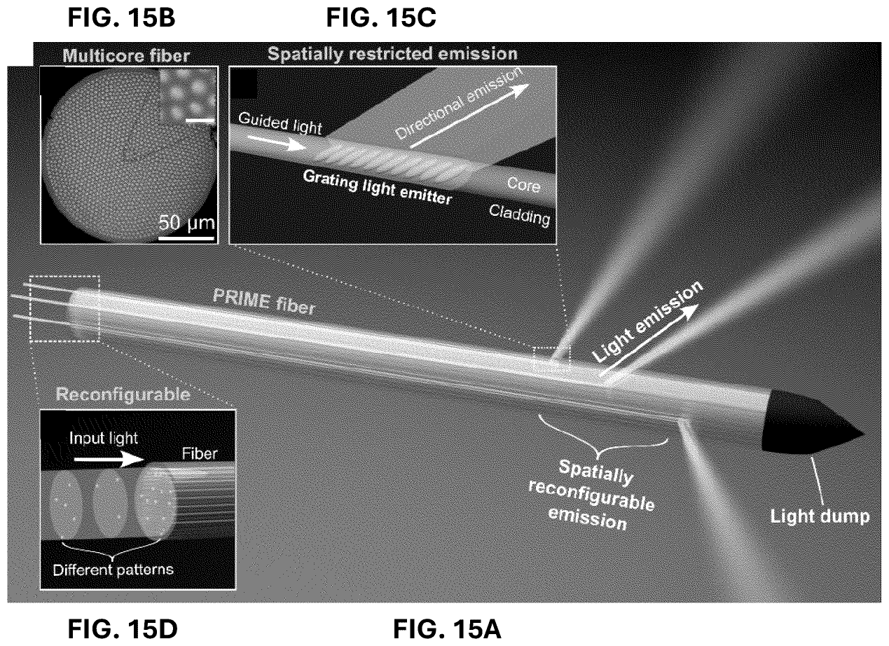

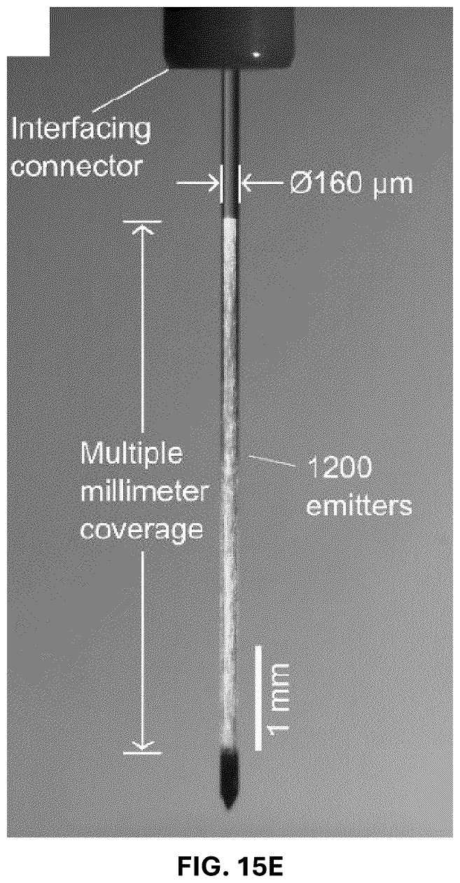

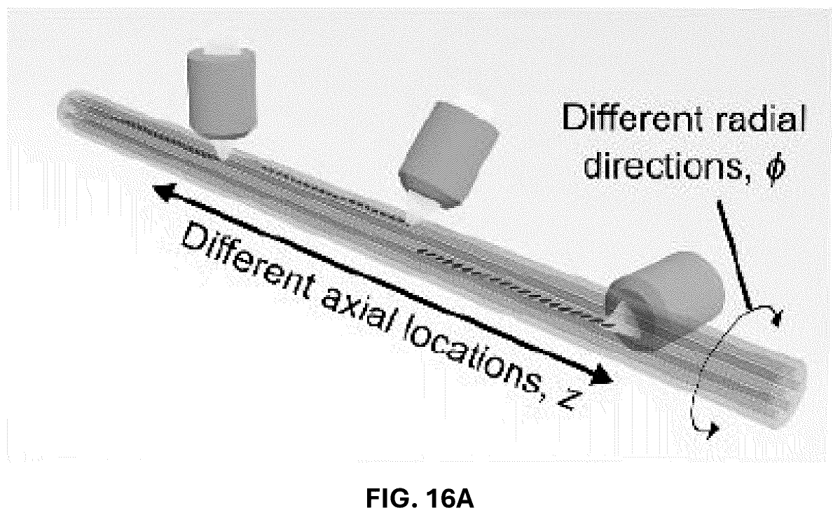

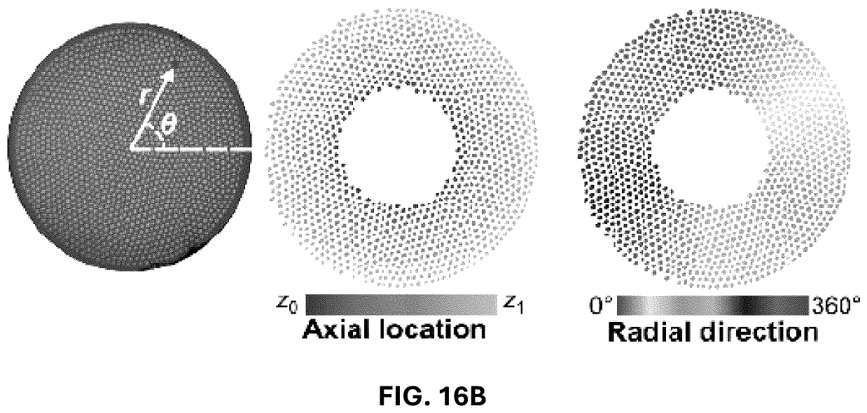

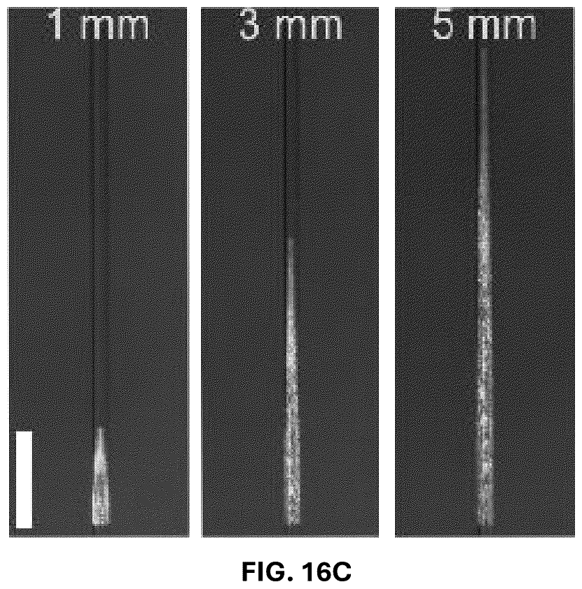

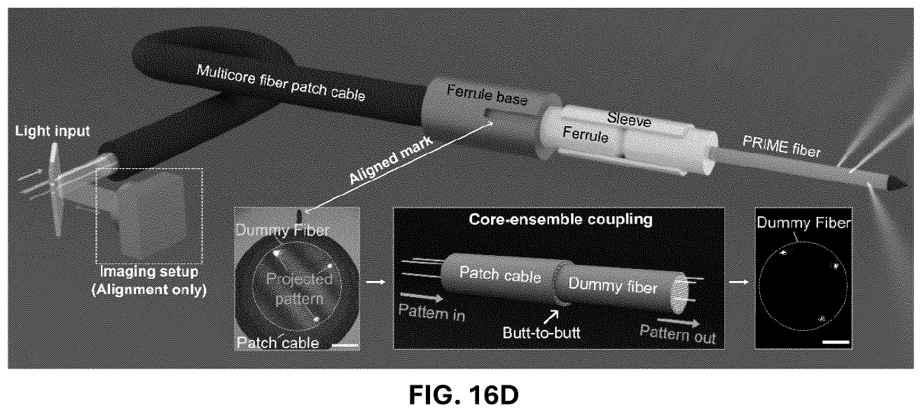

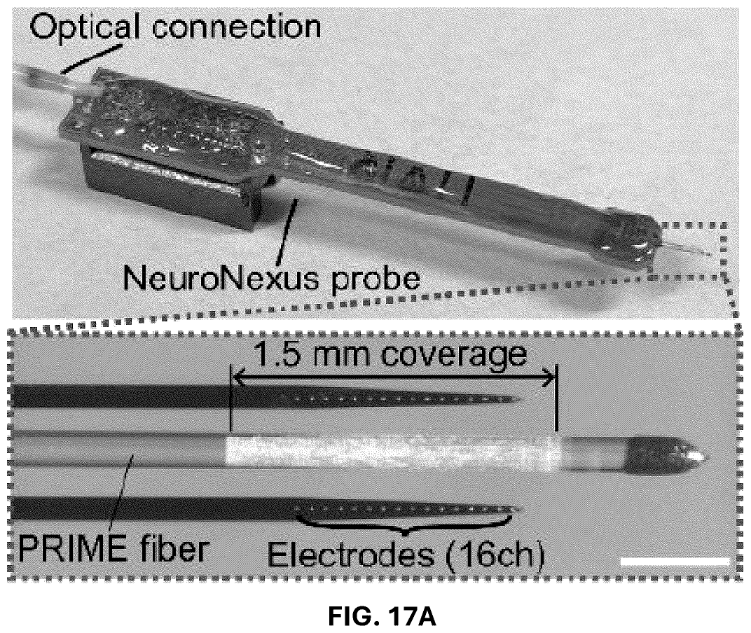

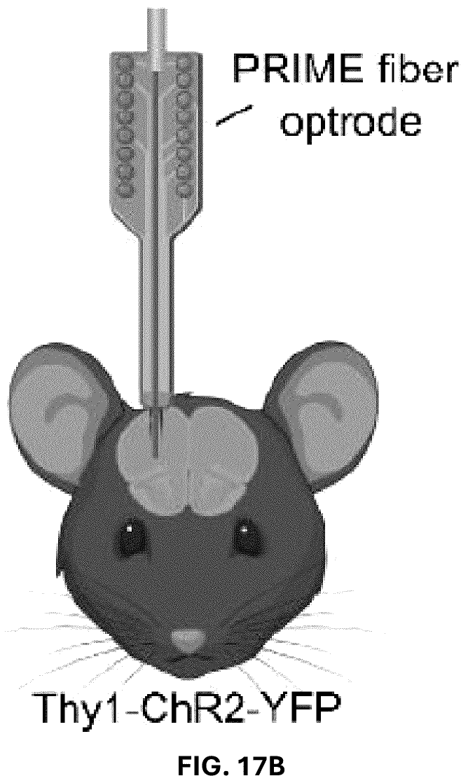

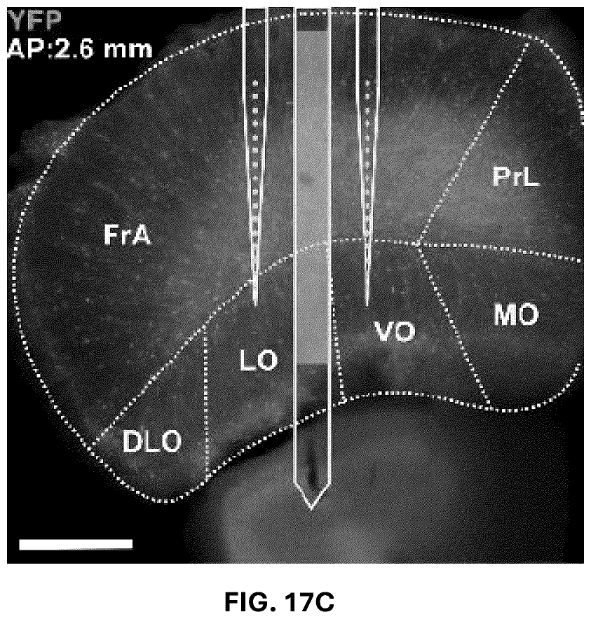

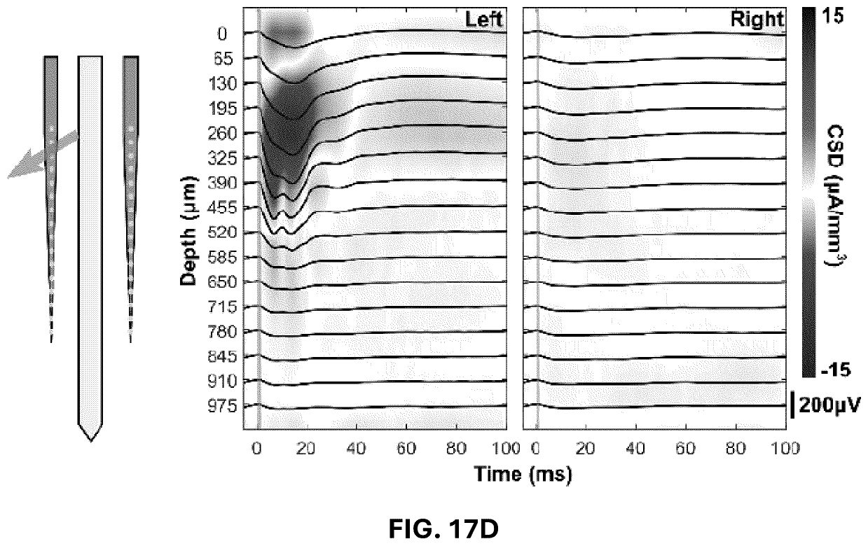

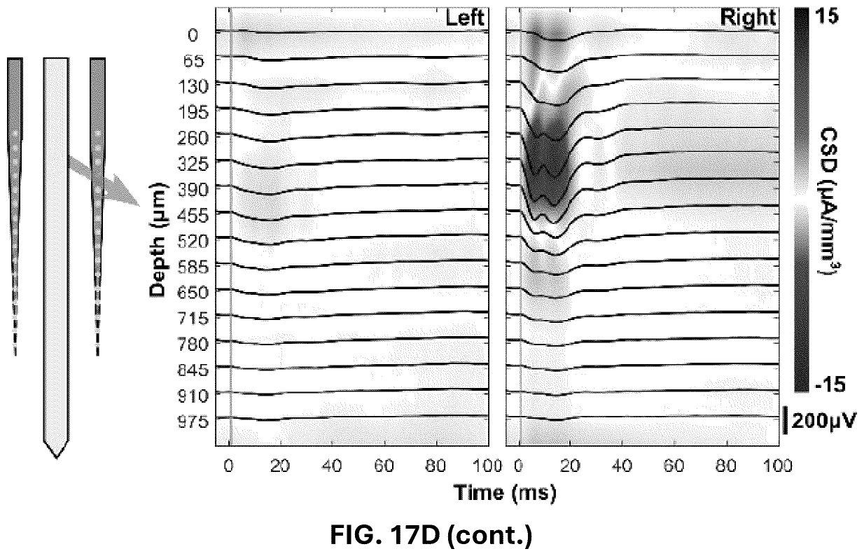

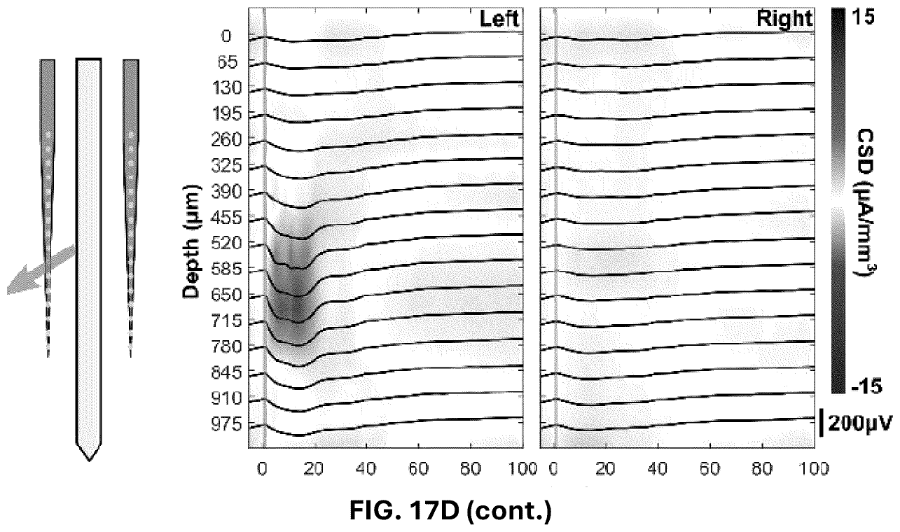

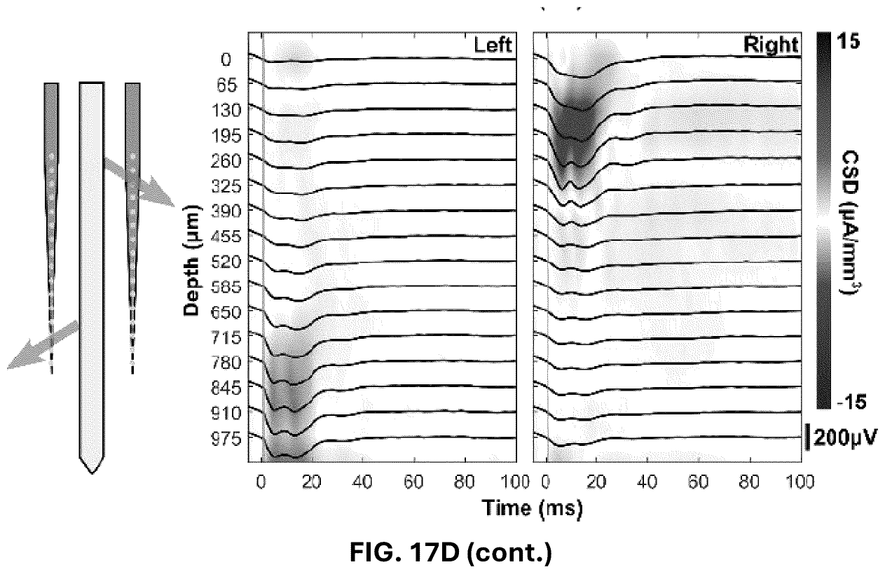

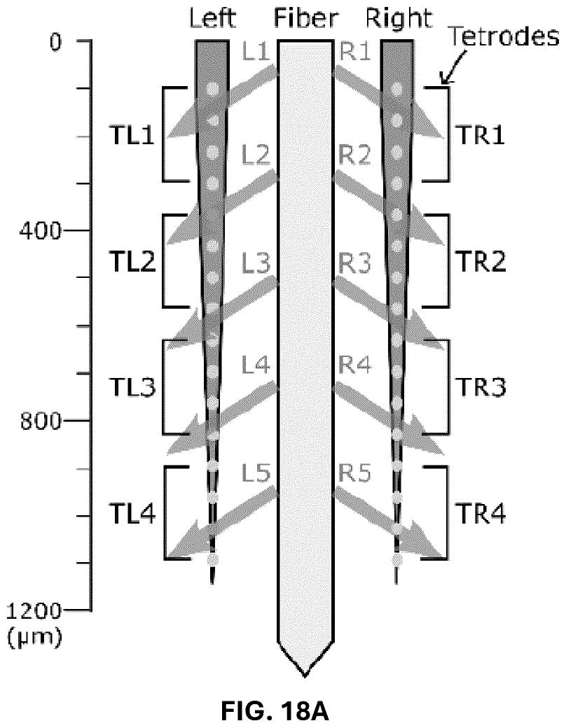

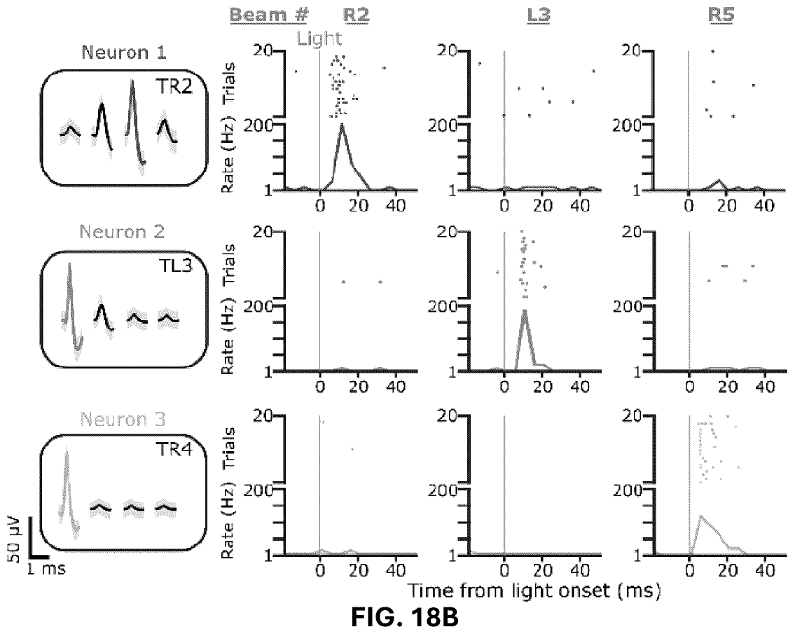

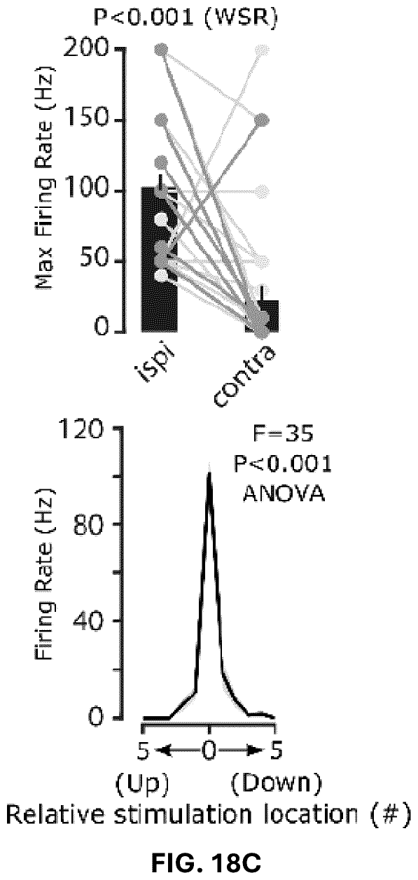

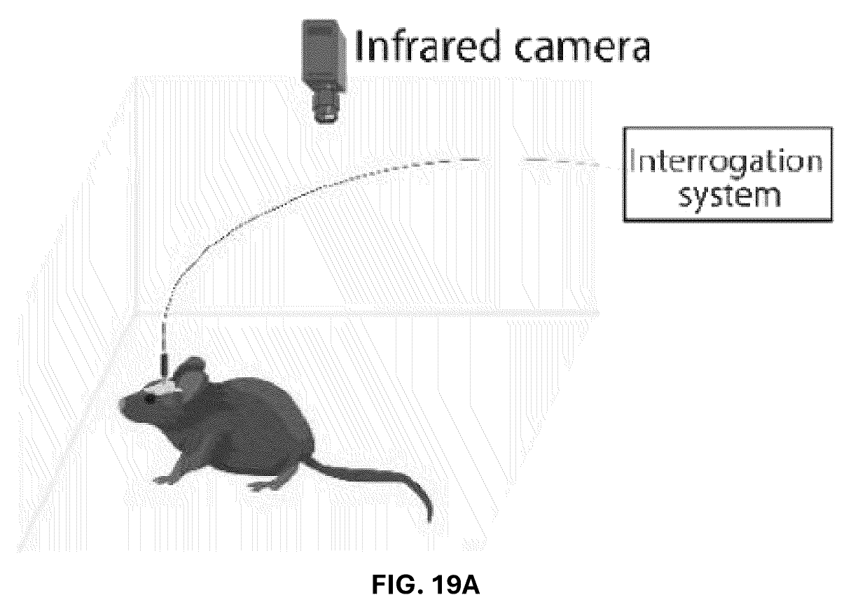

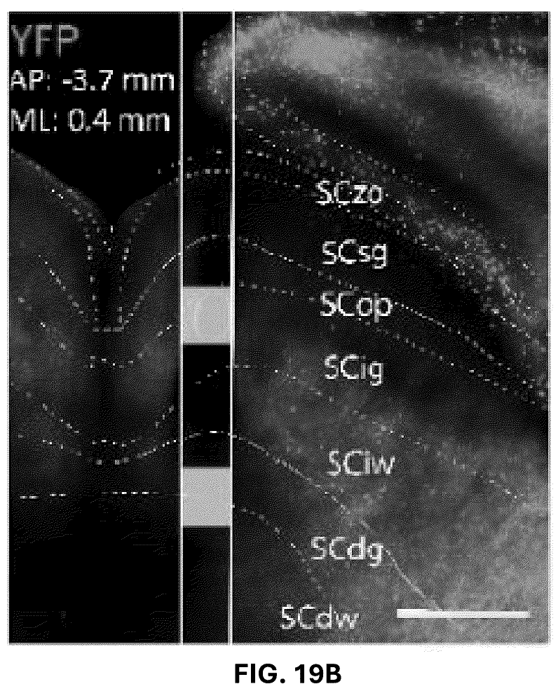

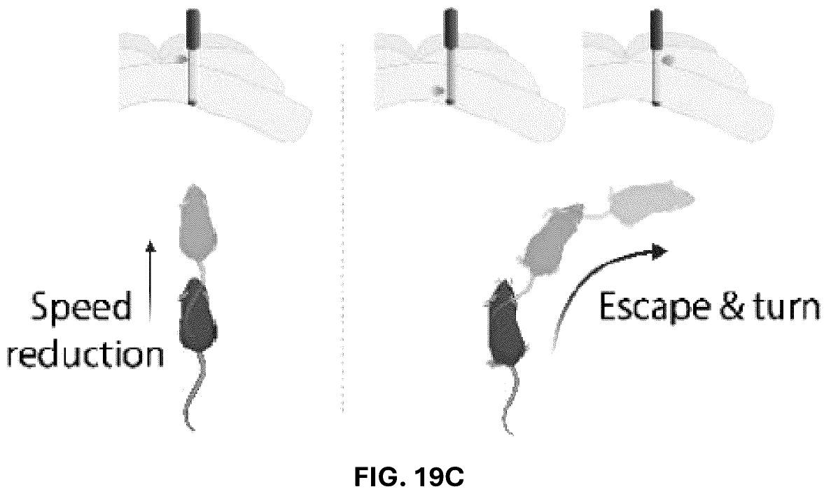

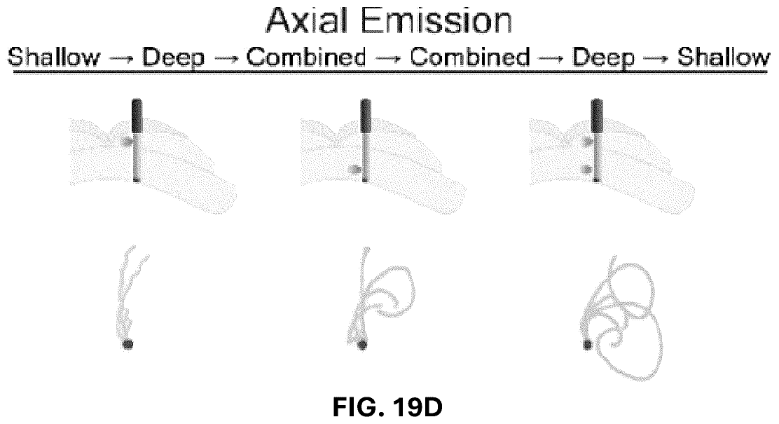

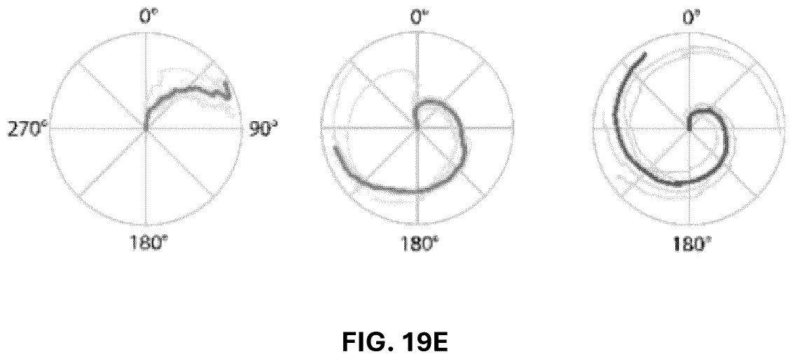

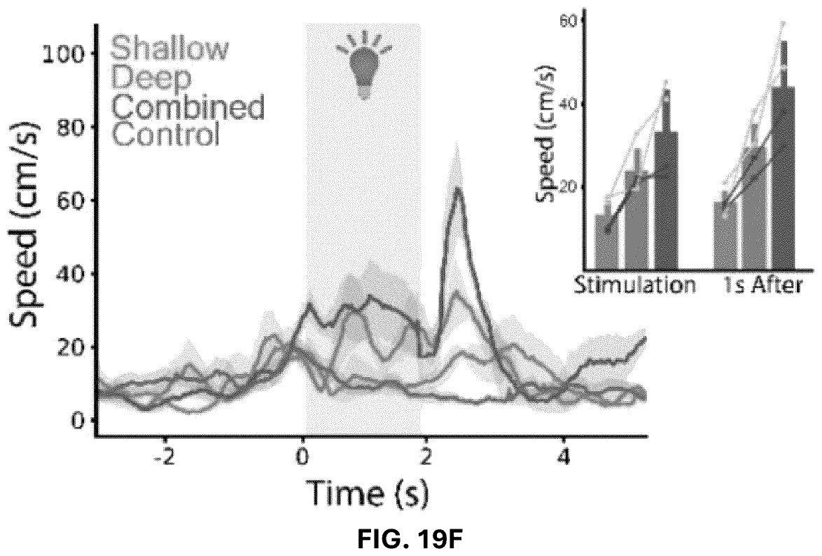

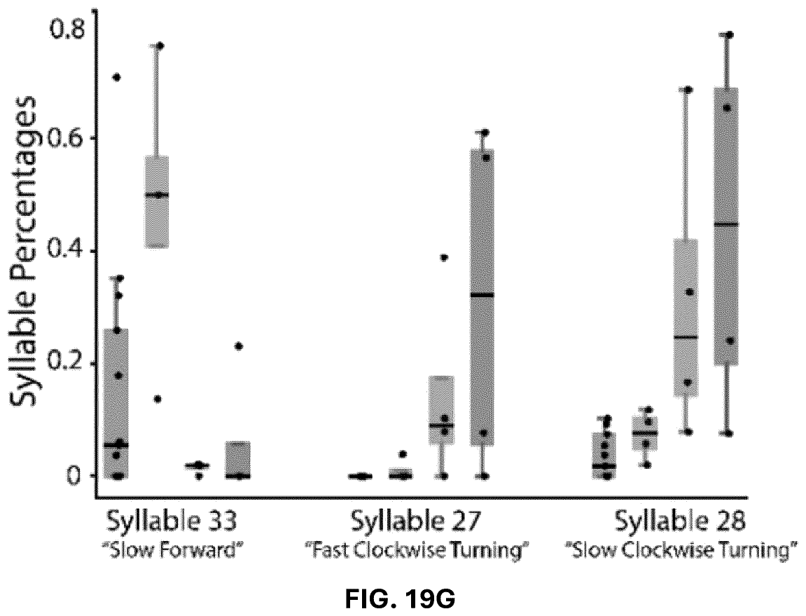

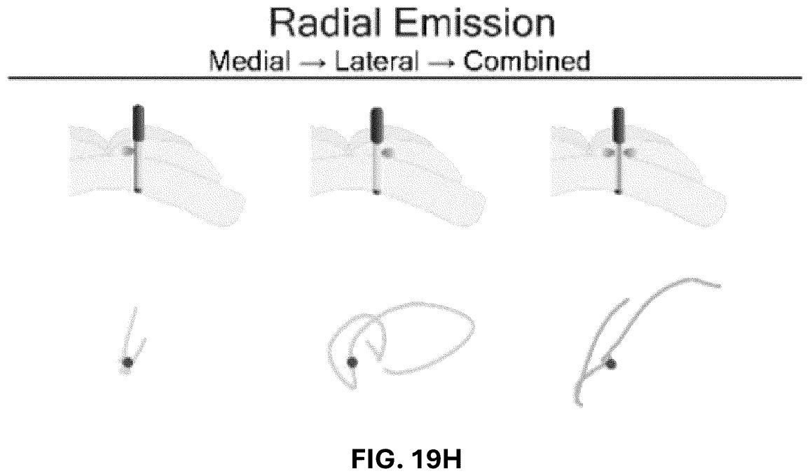

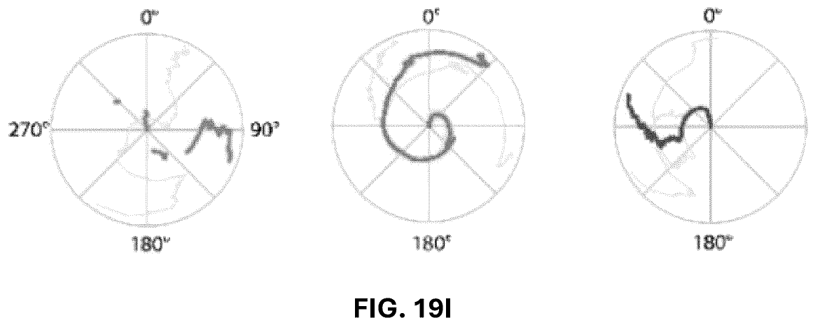

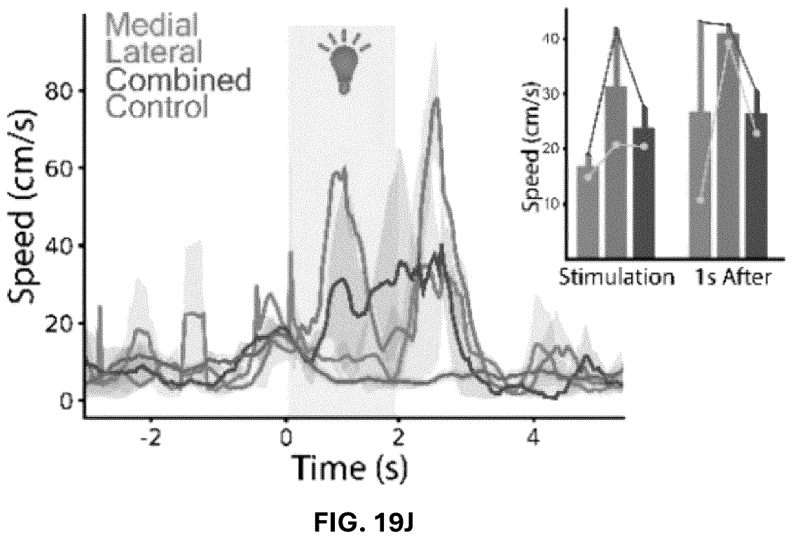

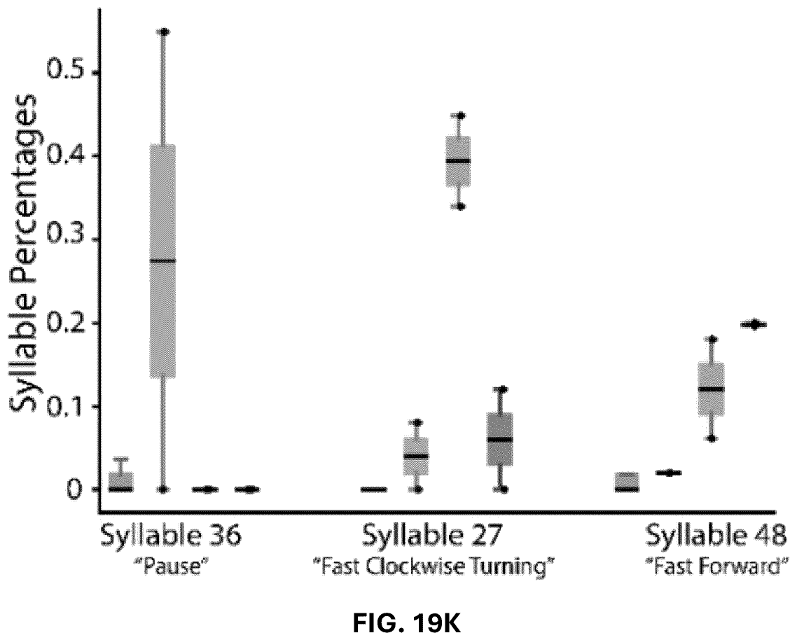

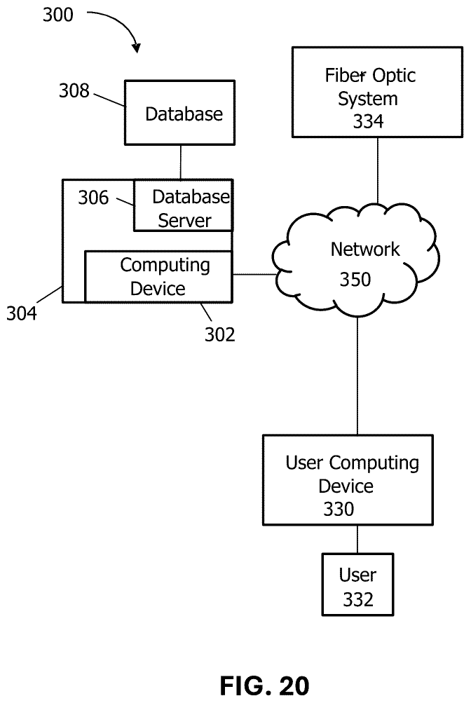

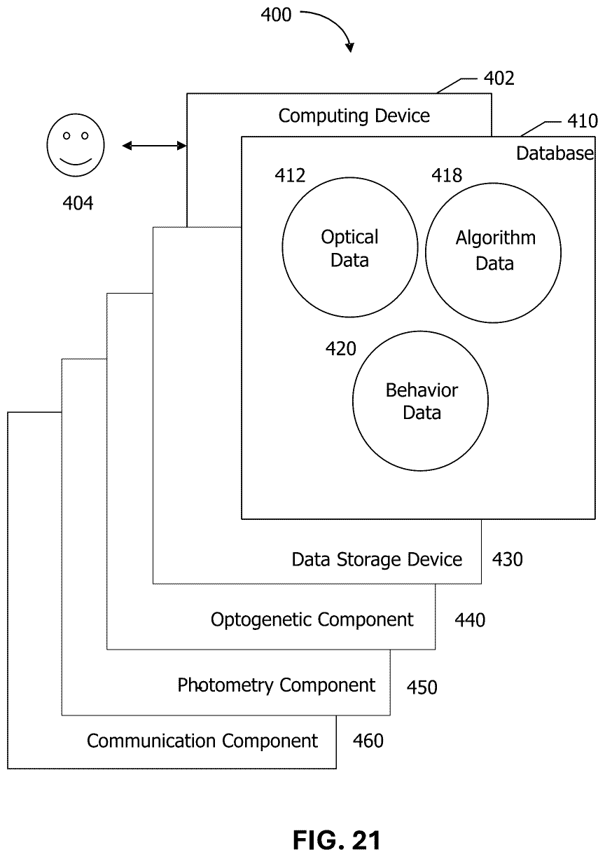

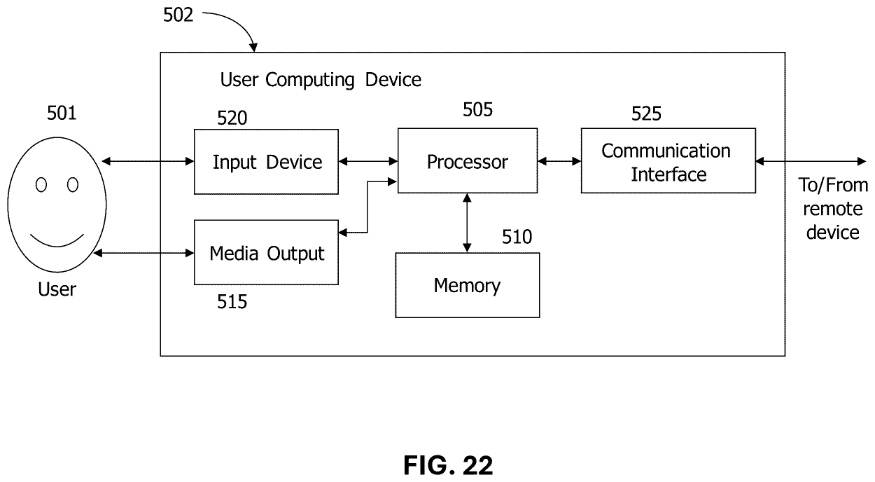

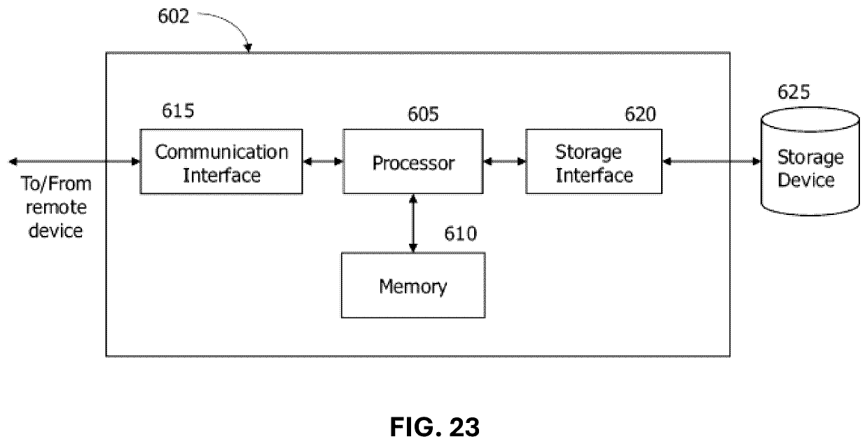

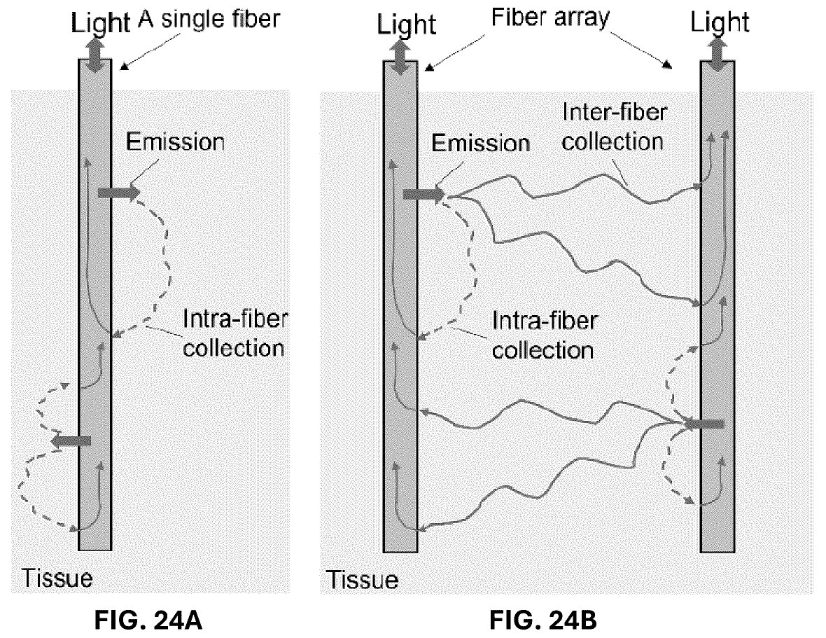

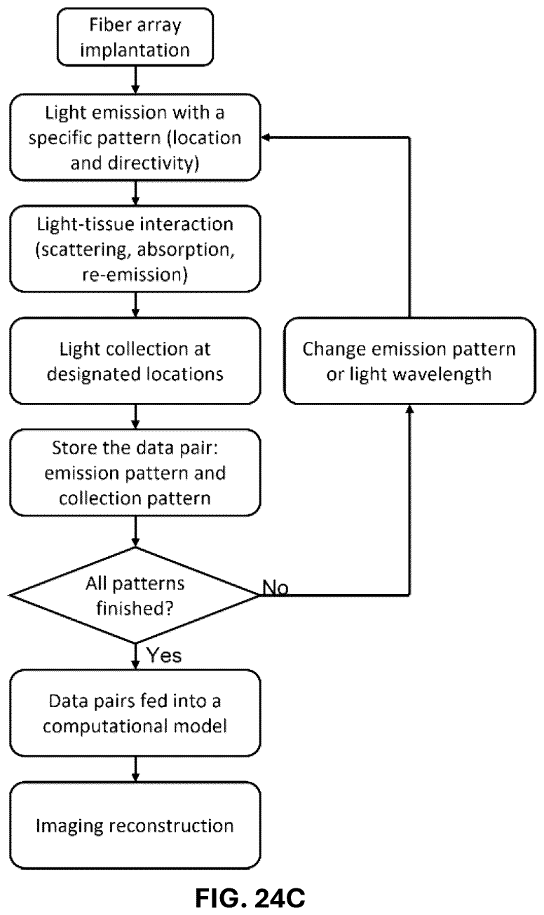

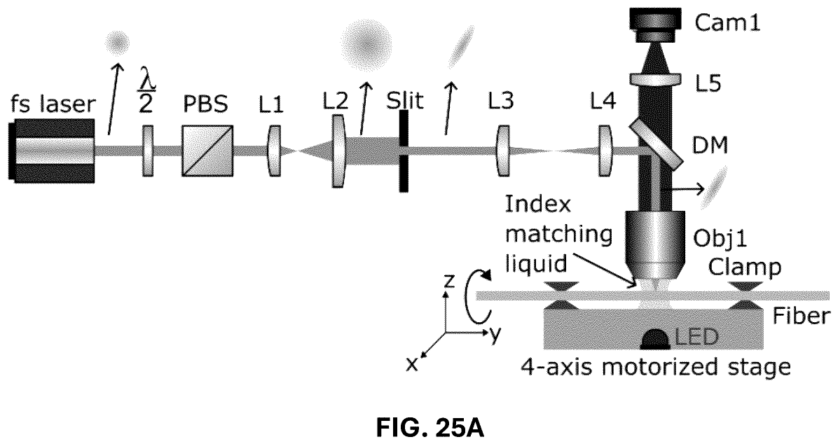

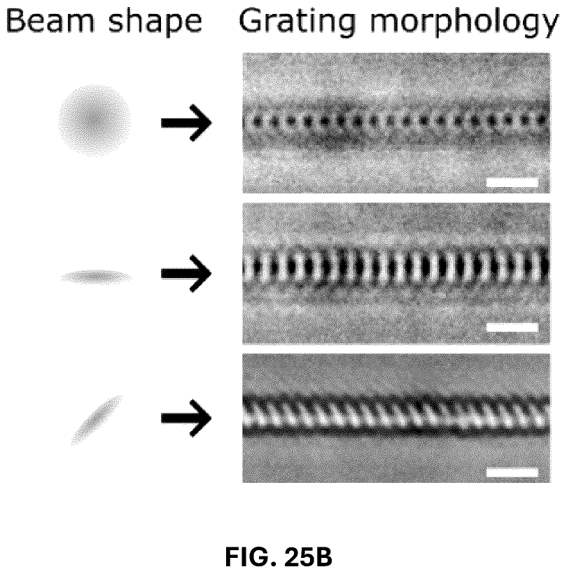

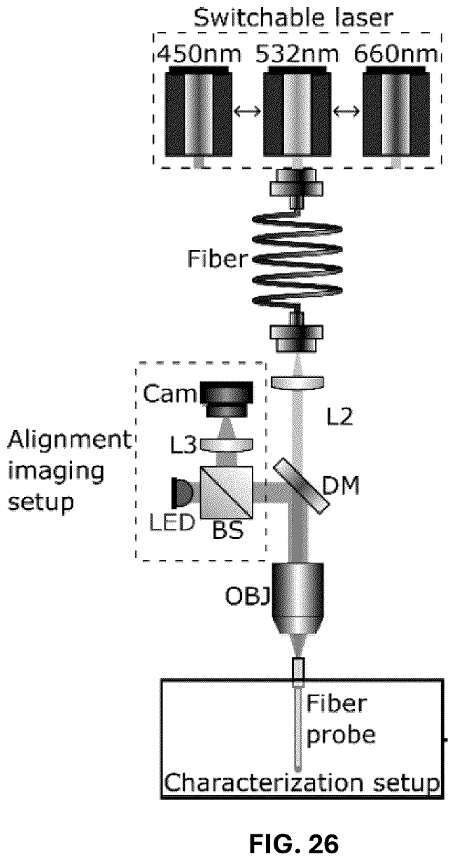

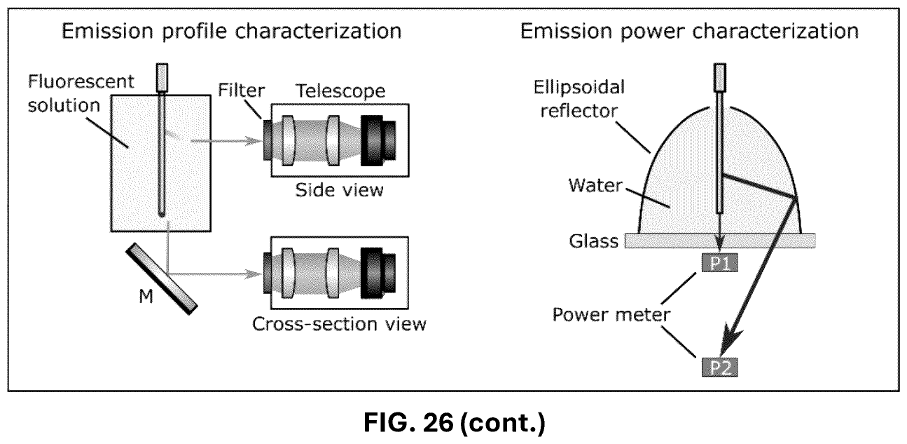

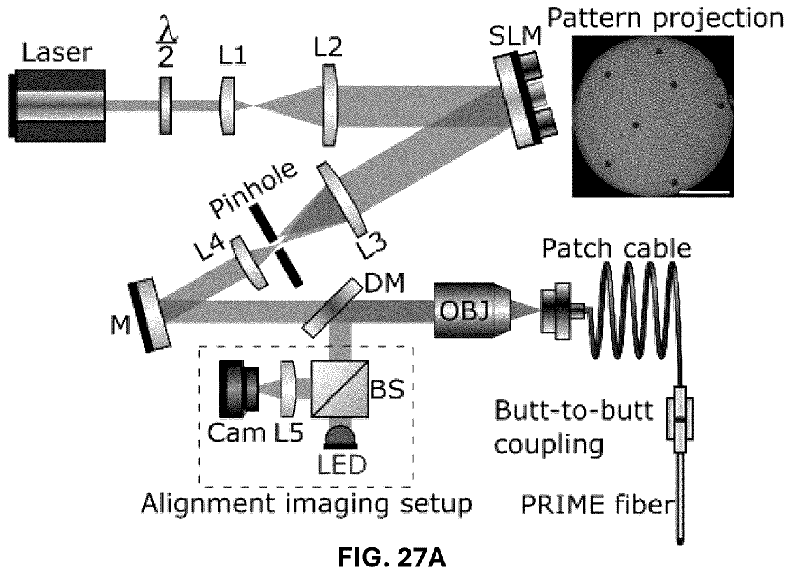

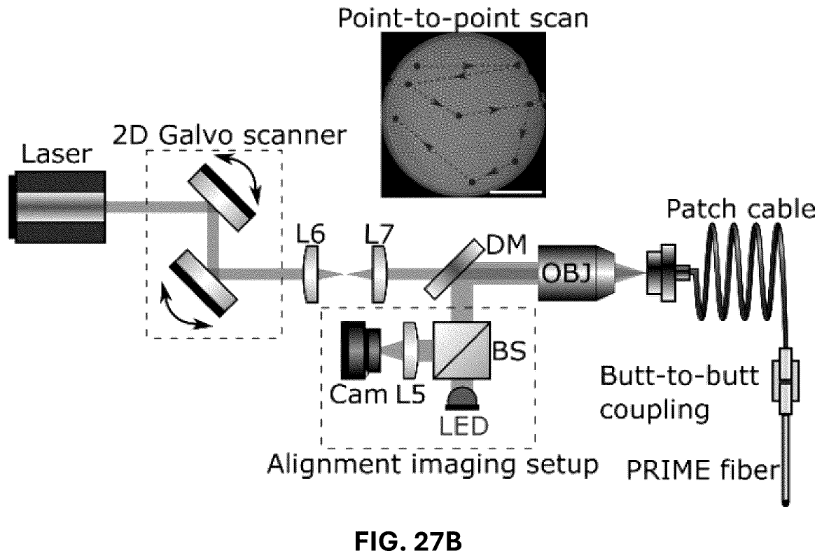

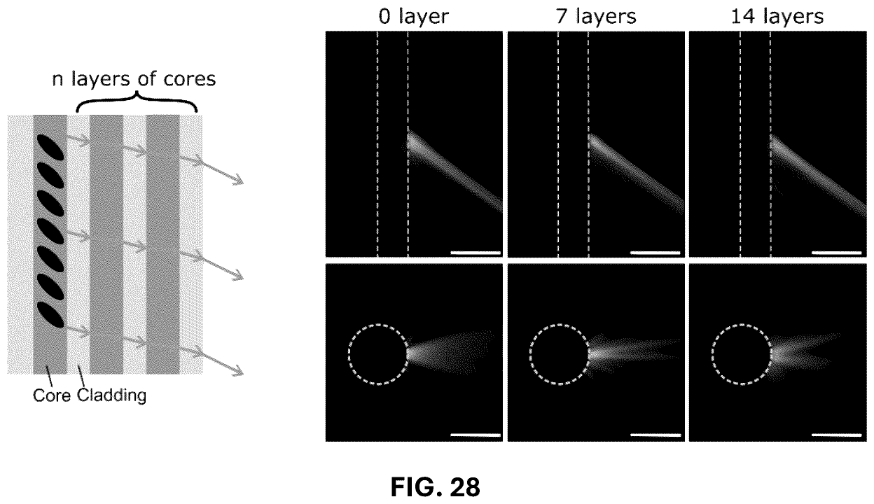

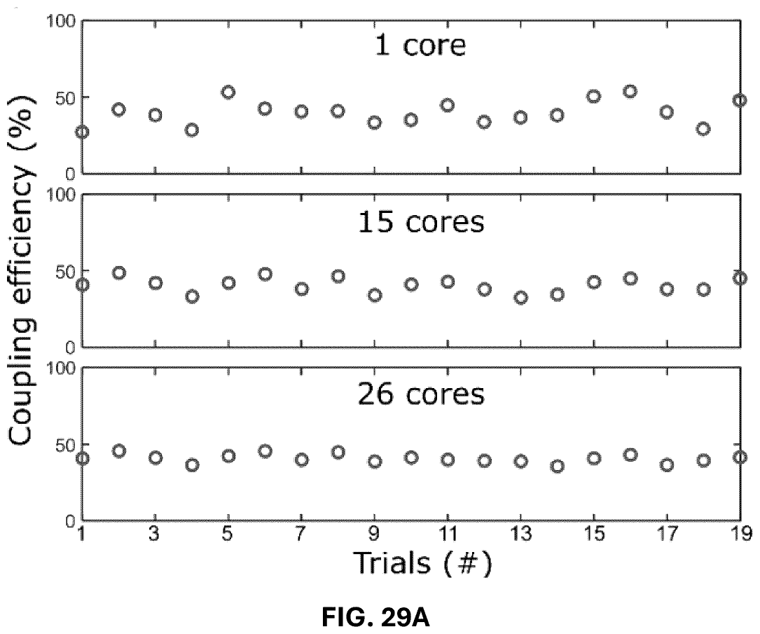

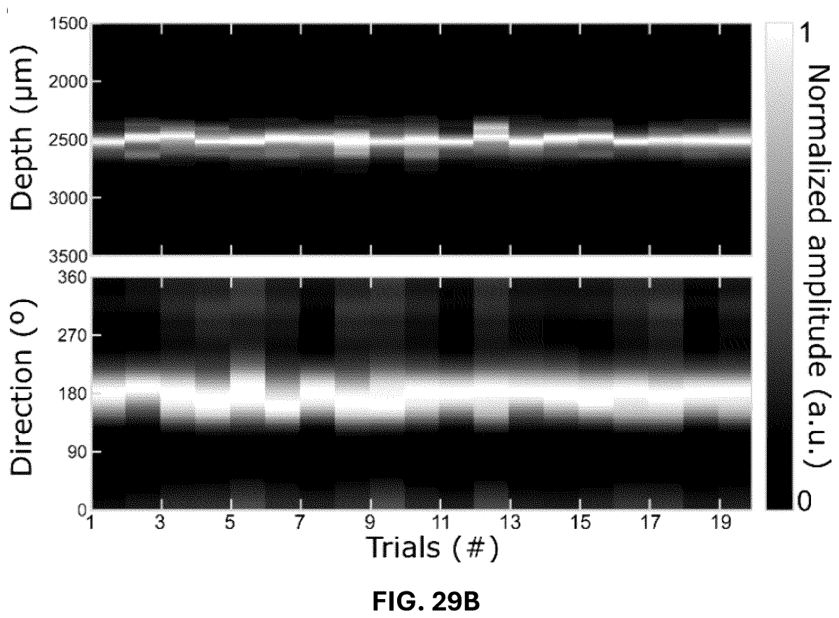

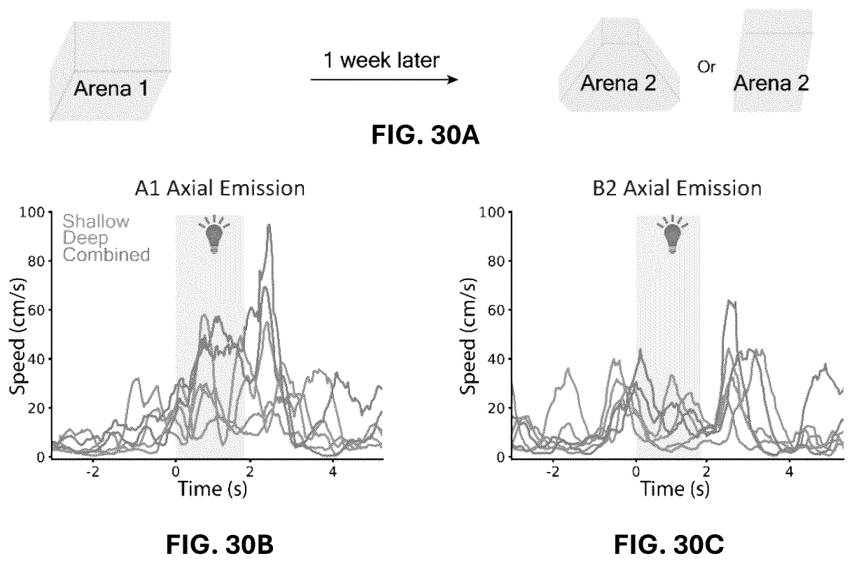

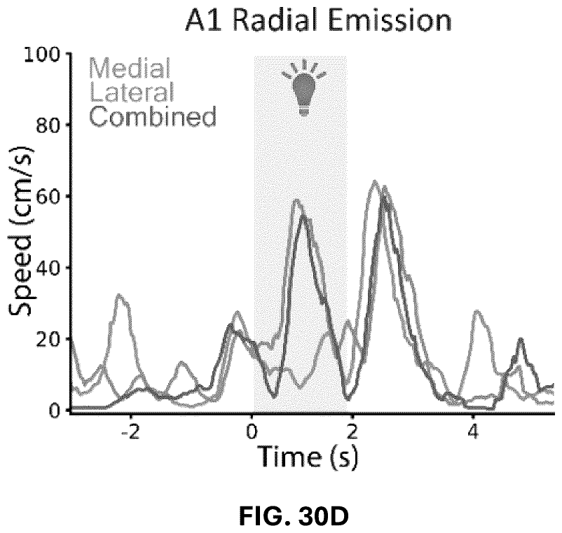

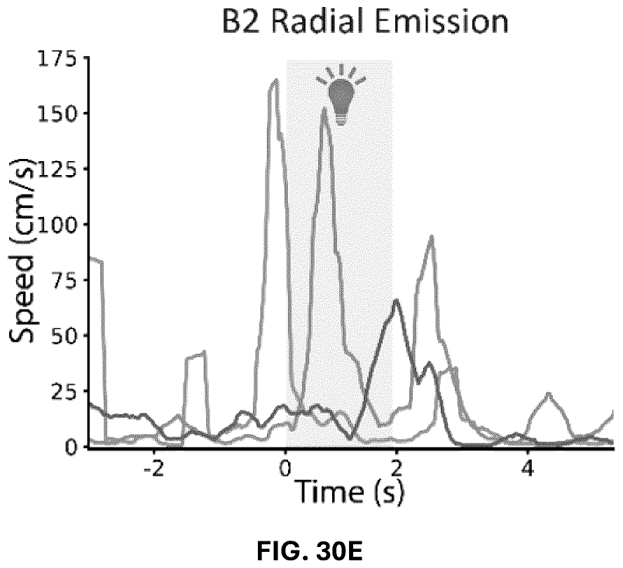

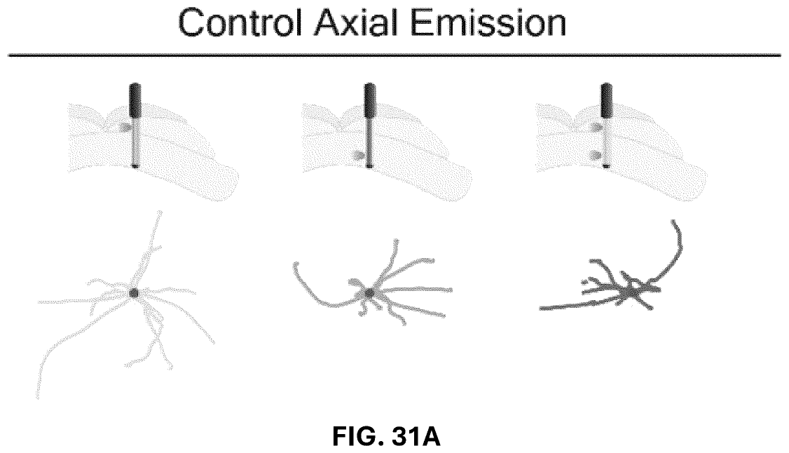

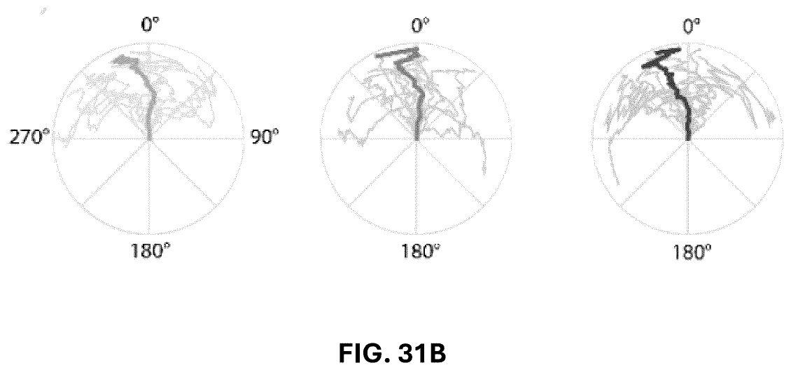

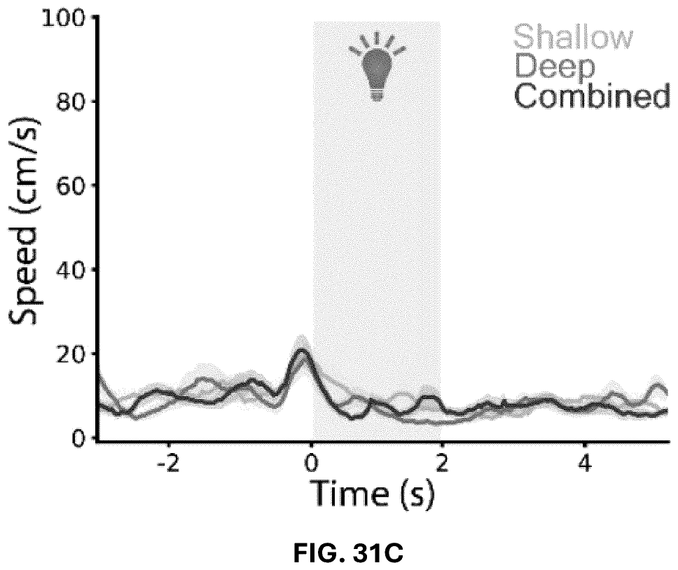

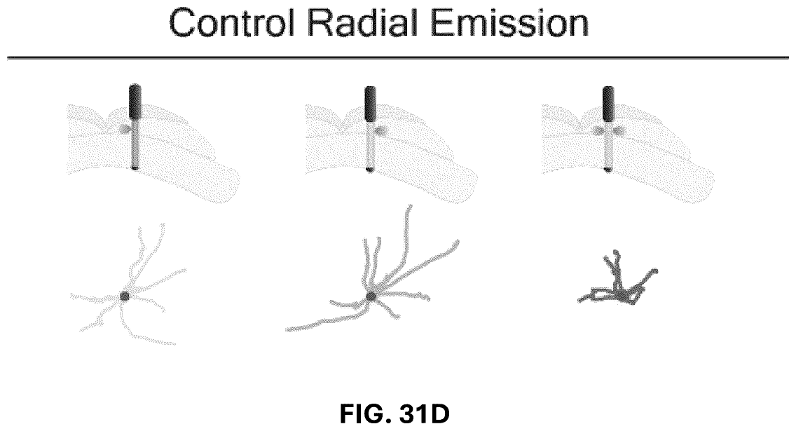

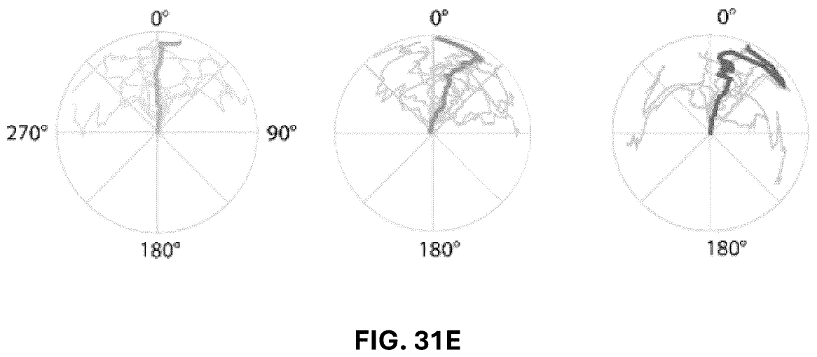

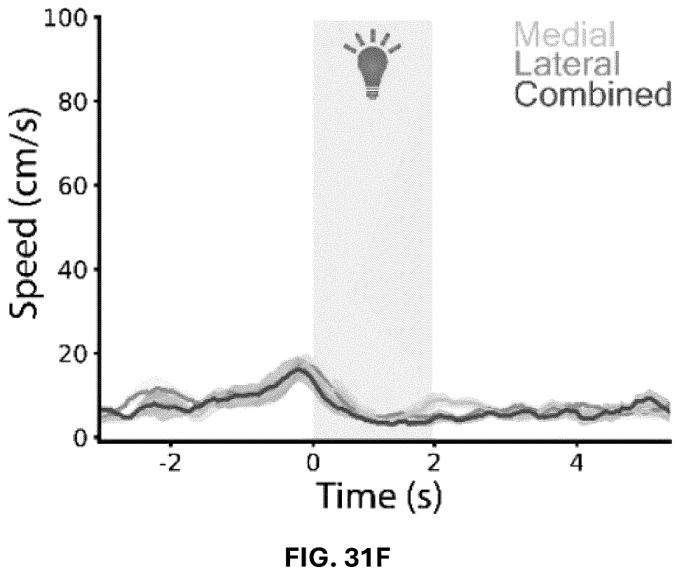

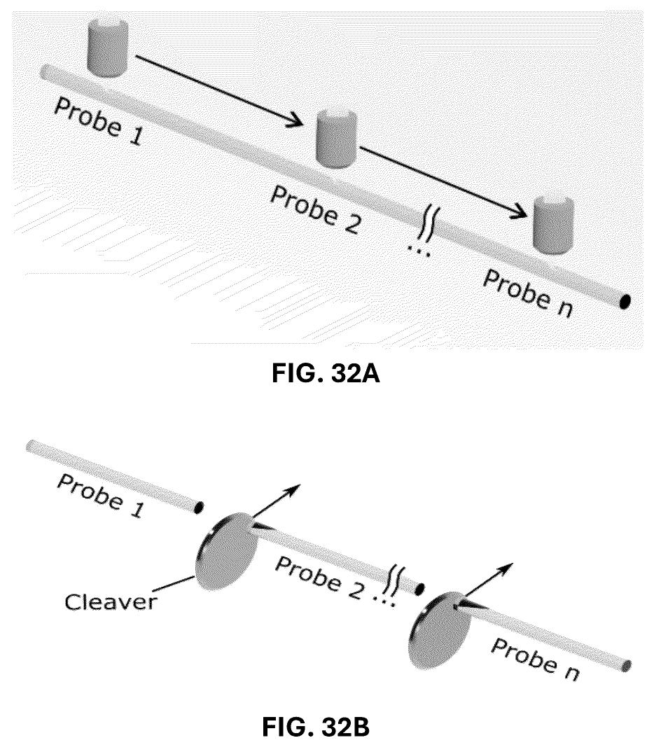

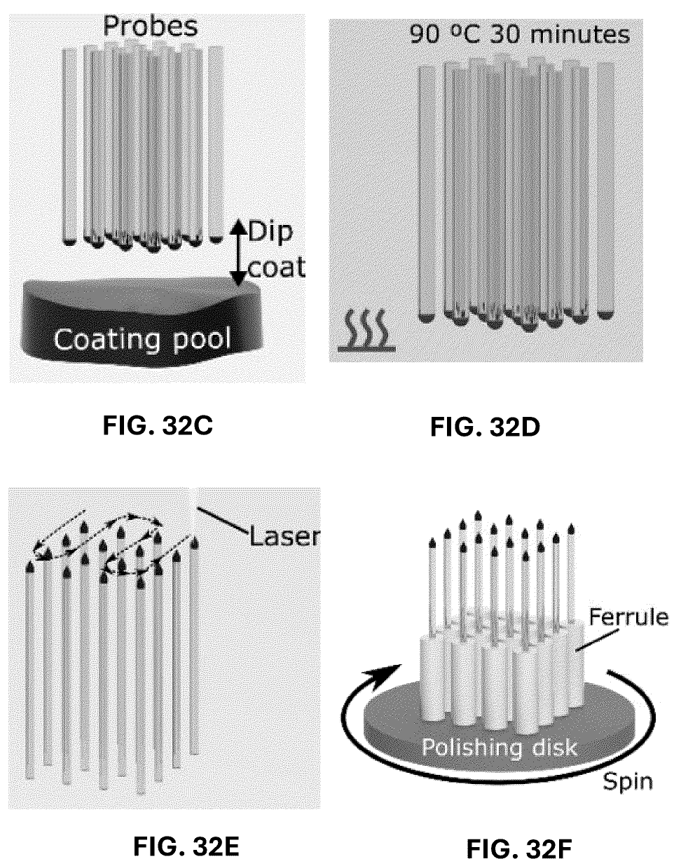

The newly designed fiber photometry is an optical fiber incorporated with two microstructures. The optical fiber can be a multimode or single-mode fiber made with glass, crystal, and/or transparent polymers. The two microstructures are in-fiber photonic crystal and multi-layer gradient filter. The in-fiber photonic crystal is a 3-dimensional array of micro-voids with engineered distribution, serving two purposes. The first is to deflect the light propagating inside the fiber core to emit sideways, perpendicular to the fiber axis, for fluorescence excitation at a selected depth. The second is to couple the fluorescence emission back into the fiber core, enabling efficient collection of the emission light entered from different angles. As shown in FIG. 7, the photonic crystal structure can be fabricated using laser micromachining, where high-energy ultrafast laser pulses are tightly focused inside the fiber and locally absorbed by the glass through a multi-photon process. Rapid absorption and deposition of the laser energy induces micro-explosion, which generates a near-vacuum void at the laser focus to provide refractive index contrast for light scattering. With precise control of the locations of laser focus, volumetric photonic crystals can be fabricated inside an optical fiber as shown in FIG. 7A. The multi-layer gradient filter is a thin-film filter, consisting of two multi-layer dielectric mirrors and a spacer in between. The thickness of the spacer is designed to gradually decrease along the fiber. The geometry of both the mirrors and the spacer can be tailored for a specific genetically encoded fluorescence indicator, including but not limited to calcium, voltage, dopamine, and norepinephrine. The structure can be fabricated with standard thin-film deposition techniques, including but not limited to physical vapor deposition, sol gel, and self-assembly. A simulation indicates that such structure offers depth-dependent narrow transmission band within the broad excitation spectrum of the indicator while maintaining high transparency over its entire emission band (FIG. 7B). Stacking those layers that operate at different wavelengths will enable depth-dependent multi-color excitation. With a wavelength-swept laser or filter, the fiber photometry is expected to achieve a depth resolution of 50-100 μm with a high temporal resolution (100-1000 Hz).

### Example 4—A Reconfigurable, Spatially Resolved Fiber-Optic Interface for Neural Stimulation

Fiber-optic technology has revolutionized neuroscience through its simplicity, affordability, high throughput, and compatibility with optogenetics and cell-type-specific targeting. Consequently, fiber-optic neural interfaces have been pivotal in deconstructing complex neural circuits, identifying behavioral and cognitive process-associated pathways, and cell types. Despite these breakthroughs, current fiber-optic neural interfaces face significant challenges including limited accessible volume and poor reconfigurability, which restrict comprehensive control over stimulation in expansive and layered brain regions. Existing modifications, such as tapered fibers and multi-fiber devices, only partially mitigate those limitations. In Example 4, laser 3D microfabrication technology was utilized to engineer a fiber-optic interface with reconfigurable light emission capability both longitudinally and radially, allowing for depth and angular-resolved stimulation. The design consists of a single 160 μm-diameter fiber-optic implant featuring 1200 independently addressable emitters spread over a 2.5 mm span and encompassing 360 degrees around the fiber. Each emitter covers an area of 150×20 μm², with a total coverage of 2500×500 μm² per fiber. This device was packaged with a standard fiber ferrule and utilized a butt-to-butt connectorization, adhering to common fiber optogenetics practices in neuroscience labs. The efficacy of this technology is demonstrated by integrating the fiber with a silicon electrode array, recording light-evoked neural activity across mouse cortical layers in vivo. Localized, light-evoked spike activity with a longitudinal resolution of 200 μm was detected, selectively confined to one or the other side of the fiber when stimulating in different directions. To evaluate the device in behaving mice, it was implanted in the superior colliculus and spatially varying light patterns were deployed. Delivering light to different layers and in different radial directions through the same fiber produced divergent behavioral responses. The findings demonstrate the potential of this laser-engineered fiber-optic interface as a versatile tool for spatially reconfigurable neural stimulations in freely behaving animals through single fibers.

### Example 5—Single Implantable Fiber Device for 2D Dynamic and Reconfigurable Light Emission and Collection

Example 5 describes various aspects and embodiments of the device of the current disclosure.

### SUMMARY

A single implantable fiber-optic device that enables dynamic and reconfigurable light emission and collection in two spatial dimensions, along and around the fiber (i.e., the longitudinal axis and the polar axis, respectively) is described. The device contains a laser-engineered optical fiber and an interrogation system (FIG. 10).

The optical fiber used for laser engineering contains multiple light-guiding cores and can be made of pristine/doped fused silica or optically transparent polymers. The diameter of the fiber ranges from tens of micrometers to a few millimeters, depending on application requirements. A short-pulsed (e.g., femtosecond or picosecond) laser beam is focused into individual light-guiding cores to modify the material's refractive index point by point. By doing so, a designed pattern of refractive index modification can be introduced to realize desired interactions with both the light propagating inside the cores and entering from the ambient environment. In turn, the light propagating inside the cores can be emitted out of the fiber, and the light from the outside can be collected into the cores. The sizes, locations, and properties (i.e., wavelength and directivity) of the light emitting and collecting sites can be precisely controlled by tailoring the laser engineering parameters, offering excellent flexibility for different applications. The morphology of the fiber tip can be flat, rounded, or tapered to accommodate different application requirements. A light-absorbing layer can be coated at the fiber tip to dump any residual light, if needed.

The interrogation system includes four parts: 1. Light source, 2. Light beam patterning, 3. Dichroic light routing, and 4. Light detection. The light source can be either incoherent or coherent depending on application requirements. The light beam patterning can be realized by a point-by-point scanning system (e.g., a galvanometer or MEMS scanner) or a pattern projection system (e.g., a spatial light modulator (SLM) or digital mirror device (DMD)). The dichroic light routing part includes lenses, dichroic mirrors, and spectral filters to separate the path of different light wavelengths. The light detection part can be a camera for 2D imaging, such as a complementary-metal-oxide-semiconductor (CMOS) or charge-coupled-device (CCD) camera, or a point detector for 1D recording, such as a photodiode and a photomultiplier tube (PMT).

### Current Progress and Implementation Examples

The ultrafast laser direct writing uses a focused femtosecond laser beam to induce multiphoton absorption within the targeted core (FIG. 11A). The highly localized photon energy deposition induces a micro-explosion that tears the glass apart, leaving a vacuum nano-void surrounded by densified glass. The significant refractive index difference between the vacuum and surrounding glass makes the nano-void optically scattering. Because the multiphoton process is confined to the focal volume, the nano-void is precisely localized within the targeted core, ensuring that the adjacent cores remain intact. This allows precise and independent modification of each core.

A single laser-written nano-void, with a tilting angle (pv) with respect to the fiber axis, induces a directional scattering of the light propagating in the core (FIG. 12A). With a periodical distribution, the directional scattering from all nano-voids is summed up coherently, and only along certain directions, which are defined as the Bragg diffraction angles (B), the scattering is enhanced via constructive light interference. ϕB can be calculated as

$${ϕ}_{B} = 90 ° - {sin}^{- 1} [ \frac{{n}_{clad}}{{n}_{a}} ( 1 - \frac{m λ}{{n}_{eff} Λ} ) ] , \qquad ( 1 )$$

where m is the diffraction order, A is the light wavelength, n_{eff} is the effective refractive index of the light propagating in the core, and A is the grating pitch. The n_{clad} and nₐ are the refractive indices of the cladding and ambient, respectively, to address the light refraction at the fiber-tissue interface. The emission spans the entire length of the grating light emitter.

To characterize the emission profile of a single grating, the fiber was immersed into a fluorescent solution and tested it using three representative wavelengths (450 nm, 532 nm, and 660 nm) in the visible range. The axial and radial emission profiles of a grating with øv=70°, A=650 nm, and L=150 μm are shown in FIG. 12B. The spatial confinement of the emission was measured by extracting the profiles of the emission intensity along the fiber axis and around the fiber circumference. As shown in FIG. 12C, the intensity profile has a the full-wave-half-maximum (FWHM) value of 41.5 μm, 35.9 μm, and 29.5 μm along the axial direction at 450 nm, 532 nm, and 660 nm, respectively. The corresponding azimuthal directionalities are 16° (450 nm), 18° (532 nm), and 21° (660 nm). Despite an inherent dispersion in the elevation angle, which can be precisely calculated and accommodated in the deployment, a single grating light emitter can achieve spatially confined directional illumination across the visible spectrum.

To demonstrate the versatile design capability, the effects of individual grating parameters on the emission properties of the grating light emitter were characterized. As indicated by Eq. (1), more than one emission directions can undergo coherent enhancement (i.e., multi-order diffraction). By controlling ϕᵥ of the nano-voids, the high-order diffraction can be suppressed. To quantify the effectiveness of suppression, the percentage of the first-order emission intensity to that of the total emission was measured with respect to different ϕᵥ (FIG. 12D). The results indicate that there are two distinct modes, single-order and multi-order emission. As long as the ϕᵥ is less than half of the ϕ_{B,min}, which is the smallest first-order Bragg diffraction angle, all three tested wavelengths operate in the mode of single-order emission. This observation indicates that a proper ϕᵥ exists for spatially confined emission at different wavelengths across the visible spectrum, despite the discrepancy in their Bragg conditions. Alternatively, a relatively large ϕᵥ can be chosen for the grating light emitter to operate in the mode of multi-order emission, in order to cover a larger volume at the expense of directionality. Also, Eq. (1) indicates that the elevation angle can be adjusted by Δ. The emission angle was measured with different Δ, which were compared to the theoretical predictions, showing an excellent agreement between the two (FIG. 12E). In this example, the diffraction angle for the best axial light confinement was maximized, and therefore Δ=650 nm, the smallest distance achievable by the current fabrication setup, was used. Finally, the dependence of the emission efficiency, η, on the grating length, L, was studied and the results were fitted with a theoretical formula, η=α·tanh²(bL), where a and b are parameters depending on the profile of the refractive index modulation inside the void and the mode field profile of the light propagating in the core. As shown in FIG. 12F, the emission efficiency initially increases with the grating lengths and then reaches a plateau, whose value is higher for shorter wavelengths. For a given grating, the light deflection efficacy correlates with the overlapping area between the light mode field and the nano-void. Therefore, the larger mode-field diameter at a longer wavelength corresponds to a lower overall efficiency due to the less interaction with the grating. Nevertheless, at least 50% of input light can be deflected out of the fiber for all three tested wavelengths.

With the interrogation systems as shown in FIG. 13A, the PRIME fiber can output confined illumination at a single axial and radial site, patterned illumination at multiple sites, or large-volume illumination covering all sites. Moreover, the illumination can be modified dynamically, as demonstrated in FIG. 13D where the illumination is switched between two distinct sites at varying frequencies (i.e., 2 Hz, 3 Hz, and 5 Hz) with a contrast ratio above 20:1.

To validate the spatially resolved light collection, a fluorescent emitter was placed at different axial positions and radial directions. The light emission scanned through the entire circumferential surface of the fiber while the light is being collected by the same fiber simultaneously. As plotted in FIG. 14, the collected light signal shows a strong correlation to the emitter position, demonstrating the technical feasibility.

### Applications

High-spatiotemporal-resolution, depth- and angularly-resolved, deep-tissue optogenetic excitation and photometric recording in the brain and other internal organs
Deep-tissue, large-field-of-view, multi-scale, optical imaging

### Example 6-Prime: Laser-Engineered Fiber for Panoramic, Reconfigurable Control of Neural Activity

### Abstract

Decoding the neural basis of behavior requires versatile control of brain-wide activity. Fiber optics enables deep-brain light delivery for neural manipulation, but precise targeting of specific regions with adaptable illumination remains challenging. To address this, PRIME (Panoramically Reconfigurable IlluMinativE), a single-fiber technique capable of emitting light from thousands of independent sites throughout its side surface, is introduced. A 160-μm multicore fiber was equipped with laser-engineered grating light emitters at designated axial and radial positions. By modulating the input light pattern, PRIME could reconfigure the illumination pattern at 60 Hz among 1,200 sites spanning 5 mm and 360°. Integration of PRIME with a high-density silicon probe enabled targeted optogenetic activation and electrophysiological recording across cortical layers in vivo. In freely moving mice, distinct defensive behaviors were induced by neural stimulation at various depths and locations in the superior colliculus using a single fiber. PRIME's reconfigurable light delivery over large volumes opens new possibilities for massively parallel optical stimulation of neural activity across brain regions.

### INTRODUCTION

Deciphering the neural processes underlying behavior requires precise, causal manipulation of neural circuits across the complex brain network. Optogenetics, combining genetic engineering with light to control neurons, has revolutionized the ability to modulate neural activity with high specificity. The advent of implantable optical technologies has further propelled optogenetics into deep-brain regions, expanding its potential for new insights. To date, two categories of these technologies have been developed at distinct spatial scales. On the one hand, miniaturized light endomicroscopy has enabled neural modulation at the cellular level, but with limited coverage (<1 mm²). On the other hand, integrated-optoelectronic probes have achieved neural modulation over millimeter scales, but with sparsely distributed emission sites (a few dozen per shank). However, there is no technology that offers precise control of the optogenetic illumination profile throughout a large brain volume, matching the abilities of electrophysiological recordings using high-density silicon probes that cover more than 1,000 sites over several millimeters. To realize the promise of optical neural control, there is a need for technologies capable of large-volume, high-density, and rapidly reconfigurable light delivery.

Optical fibers are widely used for optogenetic control of neurons, and the prominence arises from its broad-band light transmission, hair-thin footprint, and straightforward plug-and-play operation. Achieving large-scale, high-density, and reconfigurable optogenetic stimulation with a single fiber requires the abilities to (i) control light emission from independent sites on the fiber that cover a large tissue volume, (ii) confine the tissue coverage of each emission site, and (iii) alter the emission pattern to dynamically target specific locations within the tissue volume. Conventional fibers, originally designed for long-haul telecommunications, exhibit strong light confinement due to the high material homogeneity and atomic-level boundary smoothness, resulting in negligible light leakage along the fiber and a restricted illumination volume at the tip. With elegant structural modifications, surface machined fibers (by introducing light scatter) and tapered fibers (by reducing the diameter) have enabled side emission. Moreover, in tapered fibers, the emission sites along the fiber axis can be reconfigured by altering the light propagating modes. However, this technique has two limitations. First, the reconfigurability is restricted to a single dimension along the fiber axis, because the circular symmetry of light propagation modes in the tapered fiber results in isotropic side emission along all radial directions. Although physical masks can be applied to confine the emission direction, the mask location is fixed once fabricated and thus does not offer any tunability or reconfigurability. Second, light emitted from the side surface of the tapered fiber forms a cone shape and is primarily oriented towards the fiber tip, which impairs the light confinement along the axial direction. Although these limitations can be mitigated by implanting multiple fibers, the invasiveness escalates with the number of implants, which is ultimately limited to a few tens.

A fiber-optic technique, PRIME (Panoramically Reconfigurable IlluMinativE), was developed to address these challenges for high-density, large-volume optical neural control in the deep brain. A straightforward butt-to-butt coupling facilitated easy integration into existing systems for plug-and-play operation. In-vivo experiments demonstrated PRIME's capacity for selective neural modulation at precise depths and radial locations, with efficacy validated using an integrated silicon probe. Furthermore, its ability to elicit specific behavioral responses in freely moving mice through targeted activation of distinct subregions in the superior colliculus is shown.

### Results

### I. Principle of the PRIME Fiber

The PRIME technique uses a single, laser-engineered multicore fiber with thousands of independent light waveguides to create a high-density mapping between the light field entering the fiber's input end and that emitted from the fiber's side surface (FIG. 15A). These waveguides, or cores, are designed to have a minimal crosstalk in the visible spectral range, allowing each core to operate as an independent light channel (FIG. 15B). Within each core, a periodic grating pattern, termed grating light emitter, is inscribed using ultrafast laser direct writing to diffract light for side emission (FIG. 15C). The strategic arrangement of the grating light emitter within each core enables a precise control over the axial position and radial direction of the emission. By inscribing thousands of such emitters at various locations throughout the fiber, a precise, one-to-one mapping between the input and emission patterns can be established. Further, by patterning and projecting the input light into selected fiber cores, it can be diffracted for side emission with any desired pattern along the axial and radial directions. In the current design, the input pattern is generated by a spatial light modulator (SLM), which allows dynamic alteration of the input pattern to reconfigure the emission pattern in real time (FIG. 15D). A light dump is coated at the fiber's output end to absorb the residual light. The PRIME technique was demonstrated using a 160-μm multicore fiber, where 1,200 distributed, individually addressable grating light emitters were engineered to achieve 5-mm axial and 360° radial illumination from the fiber's side surface (FIG. 15E).

### II. Design and Fabrication of the Grating Light Emitter

We use ultrafast laser direct writing with a focused femtosecond laser beam (Ultrafast laser direct writing of the fiber grating emitter section below and FIG. 25) to create the grating light emitter in each targeted core of the fiber (FIGS. 11A and B). The highly localized multiphoton absorption, primarily four-photon, induces a micro-explosion that tears the glass apart, leaving a vacuum nano-void surrounded by densified glass. The significant refractive index difference between the vacuum and surrounding glass makes the nano-void optically scattering. Because the multiphoton process is confined to the focal volume, the nano-void is precisely localized within the targeted core, ensuring that the adjacent cores remain intact. This allows precise and independent modification of each core.

A non-limiting example of a suitable ultrafast laser direct writing method for forming shaped nanovoids within the optic fibers is provided in Watanabe 2001, “Femtosecond laser-assisted three-dimensional microfabrication in silica”, Optics Letters 26 (5): 277-279, the content of which is incorporated by reference herein in its entirety.

A single laser-written nano-void, with a tilting angle (ϕᵥ) with respect to the fiber axis, induces a directional scattering of the light propagating in the core (FIG. 12A). With a periodical distribution, the directional scattering from all nano-voids is summed up coherently, and only along certain directions, which are defined as the Bragg diffraction angles (ϕ_{B}). This scattering is then enhanced via constructive light interference. ϕ_{B} can be calculated as

$${ϕ}_{B} = 90 ° - {sin}^{- 1} [ \frac{{n}_{clad}}{{n}_{a}} ( 1 - \frac{m λ}{{n}_{eff} Λ} ) ] , \qquad ( 1 )$$

where m is the diffraction order, A is the light wavelength, n_{eff} is the effective refractive index of the light propagating in the core, and A is the grating pitch. The n_{clad} and nₐ are the refractive indices of the cladding and ambient, respectively, to address the light refraction at the fiber-tissue interface. The emission spans the entire length (L) of the grating light emitter.

To characterize the emission profile of a single grating, the fiber was immersed into a fluorescent solution (Methods and FIGS. 26 and 27) and tested it using three representative wavelengths (450 nm, 532 nm, and 660 nm) in the visible range. The axial and radial emission profiles of a grating with ϕᵥ=70°, Δ=650 nm, and L=150 μm are shown in FIG. 12B. The spatial confinement of the emission was measured by extracting the profiles of the emission intensity along the fiber axis and around the fiber circumference. As shown in FIG. 12C, the intensity profile has a the full-wave-half-maximum (FWHM) value of 41.5 μm, 35.9 μm, and 29.5 μm along the axial direction at 450 nm, 532 nm, and 660 nm, respectively. The corresponding azimuthal directionalities are 16° (450 nm), 18°(532 nm), and 21° (660 nm). Despite an inherent dispersion in the elevation angle, which can be precisely calculated and accommodated in the deployment, a single grating light emitter can achieve spatially confined directional illumination across the visible spectrum.

To demonstrate the versatile design capability, the effects of individual grating parameters on the emission properties of the grating light emitter were characterized. As indicated by Eq. (1), more than one emission directions can undergo coherent enhancement (i.e., multi-order diffraction). By controlling ϕᵥ of the nano-voids, the high-order diffraction can be suppressed. To quantify the effectiveness of suppression, the percentage of the first-order emission intensity to that of the total emission with respect to different ϕᵥ was measured (FIG. 12D). The results indicate that there are two distinct modes, single-order and multi-order emission. As long as the ϕᵥ is less than half of the ϕ_{B,min}, which is the smallest first-order Bragg diffraction angle, all three tested wavelengths operate in the mode of single-order emission. This observation indicates that a proper ϕᵥ exists for spatially confined emission at different wavelengths across the visible spectrum, despite the discrepancy in their Bragg conditions. Alternatively, a relatively large ϕᵥ can be chosen for the grating light emitter to operate in the mode of multi-order emission, in order to cover a larger volume at the expense of directionality. Also, Eq. (1) indicates that the elevation angle can be adjusted by Δ. The emission angle was measured with different Δ and compared with the theoretical predictions (see Methods), showing an excellent agreement between the two (FIG. 12E). In this work, the researchers chose to maximize the diffraction angle for the best axial light confinement, and therefore Δ=650 nm, the smallest distance achievable by the current fabrication setup, was used. Finally, the dependence of the emission efficiency, η, on the grating 135 length, L, was studied (FIG. 26). Because the emission can be treated as the loss of the light propagating in the core, the results were fitted with a formula, η=ηₘₐₓ. (1−e^{-κL}), where ηₘₐₓ and κ are the maximum efficiency and loss coefficient, respectively. As shown in FIG. 12F, the emission efficiency initially increases with the grating lengths and then reaches a plateau, whose value is higher for shorter wavelengths. For a given grating, the light deflection efficacy correlates with the overlapping area between the light mode field and the nano-void.

Therefore, the larger mode-field diameter at a longer wavelength corresponds to a lower overall efficiency due to the less interaction with the grating. Nevertheless, at least 50% of input light can be deflected out of the fiber for all three tested wavelengths.

### III. Design and Interrogation of the PRIME Fiber

To achieve tailored emission from the PRIME fiber, the grating light emitters were inscribed at designated locations of individual fiber cores, enabling side emission from user-defined axial positions and radial directions (z and $ in FIG. 16A, respectively). While the emitted light from the inner cores needs to go through the outer ones, the results show that the emission maintains both the axial and directional confinements after passing through up to 14 layers of cores (˜46 μm thick), with only slight intensity modulation along the circumference (FIG. 28). Based on this observation, a symmetrical design was implemented to distribute the grating light emitters. By defining the location of each core in the polar coordinate system with a radius r and a polar angle q, the axial and radial locations of the grating light emitter can be expressed as

$$z = {z}_{0} + α r \qquad ( 2 )$$

$${r}_{0} \leq r \leq R ,$$

$$ϕ = θ$$

$$0 \leq θ < 2 π ,$$

where z₀ is the starting axial position, a is the resolution factor, and r₀ and R are the starting and end radiuses, respectively. The axial and radial distributions of the grating light emitters (r₀=30 μm, R=75 μm) are illustrated in FIG. 16B. The resolution factor, α, in Eq. (2) determines the axial resolution and coverage, and a larger a results in a wider axial coverage at the expense of resolution. Examples with axial coverages of 1 mm, 3 mm, and 5 mm are shown in FIG. 16C. This design has two advantages: (1) light emission from each core travels the shortest distance to the fiber's side surface, thereby minimizing the influence from the outer cores and maintaining spatial confinement; and (2) the rotational symmetry only requires a reference radial direction during the alignment, thereby facilitating practical implementation. Despite utilizing only the outermost 14 layers of cores, this design still incorporates 1,205 cores. After the ultrafast laser direct writing, the fiber was packaged inside a standard 1.25-mm ferrule with a taper-shape light dump fabricated at the fiber's distal end to absorb the residual light (Methods and FIG. 15E).

The interrogation method of the probe is illustrated in FIG. 16D (see FIG. 27 for detailed setups). To facilitate in-vivo applications, the PRIME fiber is designed to have a butt-to-butt connection to a lead-in patch cable, by following the common practice for fiber-based optogenetics. The patch cable is made of a multicore fiber for light pattern relay. A designed 2D light pattern is generated by a SLM (FIG. 27A) and projected onto the input end of the patch cable. Due to manufacturing variations in the fiber diameter (+15 μm) and ferrule dimension (+1.25 μm), precise core-to-core alignment is challenging. Therefore, a core-ensemble coupling strategy was introduced: using a small group of adjacent cores, rather than a single core, as an emission site. Since the location of the emission site is defined by the centroid of the core group, this strategy does not reduce the site count. The 2D light pattern is transmitted through the patch cable, resampled by the PRIME fiber at the butt-to-butt interface, and input into the designated cores of the fiber. To examine the efficacy of the core-ensemble coupling strategy, a pattern selectively illuminating three regions was projected onto the patch cable, and the output pattern was recorded at the distal end of a PRIME fiber that had neither grating light emitters inside nor a light dump at the end (FIG. 16D). The structural similarity index (SSIM) between the input and output patterns is 0.85. Further examinations reveal that increasing the number of cores per group improves the consistency of power coupling efficiency because of the averaging effect (FIG. 29A). In our in vivo studies, adjacent cores were grouped to balance the consistency of the coupling efficiency and the size of the emission site.

To precisely align the fiber's input with the patch cable's output, marks were laser-engraved on them to designate their orientations and avoid confusion induced by the twist of the patch cable (FIG. 16D). The laser marks also serve as a reference for the radial direction once connected. Overall, the symmetric core mapping rules and the core-ensemble coupling strategy allow for a convenient connection of the patch cable and the PRIME fiber without the need for a precise core-to-core alignment. Some longitudinal studies may require connecting and using the same probe at different time points. Thus, the target sites should have minimum spatial shifts each time. The repeatability was tested by connecting and disconnecting a PRIME fiber multiple times and recording the emission profiles in both the axial and radial directions with the same light input (FIG. 29B). The results show that the maximum position and direction error are +41 μm and +10°, respectively, which are only 3.3% (82 μm/2,500 μm) and 5.6%) (20°/360° of the axial and radial coverage of the PRIME fiber.

The emission pattern can be reconfigured in the same fiber by simply generating different 2D input light patterns on the remote end. As shown in FIG. 13C, the PRIME fiber can output confined illumination at a single axial and radial site, patterned illumination at multiple sites, or large-volume illumination covering all sites. Moreover, the illumination can be modified dynamically, as demonstrated in FIG. 13D where the illumination is switched between two distinct sites at varying frequencies (i.e., 2 Hz, 3 Hz, and 5 Hz) with a contrast ratio above 20:1. The dynamic reconfigurability is further showcased in two videos. Specifically, one video demonstrates single-site, multi-site, and large-volume illuminations using a single PRIME fiber. Another video demonstrates the PRIME fiber's programmability by converting it into a “piano”, where the 0° and 180° emissions correspond to the left and right hands, respectively, and the axial locations correspond to the octave notes.

### IV. Spatially Resolved Maneuvering of Neural Activity In Vivo

Integrating the PRIME fiber with optogenetics allows for targeted neural modulation at specific axial and radial positions of a single implant. For practical in vivo use, we integrated the PRIME fiber with a NeuroNexus silicon probe, enabling simultaneous optogenetic stimulation and electrophysiological recording (FIG. 17A). This integrated optrode was acutely implanted into the frontal cortex of Thy1-ChR2-YFP mice under anesthesia (FIGS. 17B and C). This cortical site was chosen because it has a relatively homogeneous distribution of ChR2-expressing neurons along the PRIME fiber's axial and radial dimensions. FIG. 17D shows that the evoked neural electrical activities align precisely with the optogenetic stimulation patterns. For single-site stimulations, laser onset induced responses of the local field potential (LFP) at corresponding cortical locations, as indicated by a negative deflection in the nearest five electrodes. These LFP responses were spatially and directionally constrained, as evidenced by the lack of deflection in electrodes at distant depths or on the opposite side. Current source density (CSD) analysis further supports that the current sinks, indicative of local depolarization, are spatially aligned with the illumination sites. Also, it was demonstrated that the PRIME fiber could simultaneously stimulate multiple sites by evoking neural activities at two distant frontal cortex locations. Again, the LFP and CSD profiles confirm the effectiveness and spatial confinement of the light emission at both sites (FIG. 17D). Notably, “w-shaped” LFP responses spanning a 20-ms time window were observed at the current sink after stimulation onset, suggesting that the evoked responses may arise from both direct and indirect activation of neighboring neurons.

The spatial confinement of the PRIME fiber-based optogenetics was further characterized at the single-unit resolution (FIG. 18A). Out of the 93 well-isolated units recorded by the optrode, 46 units showed an increase in firing rate directly following the light onset. Spike rasters and peri-stimulus time histograms show that single units on different shanks or axial positions were actively recruited only when the light targeted their respective locations (FIG. 18B). Although possible indirect, network effects could not be ruled out, it was found that the optogenetic stimulation from the PRIME fiber to be both laterally and axially specific (FIG. 18C). On the one hand, less than 30% of the light-responsive units on one shank responded to a contralateral stimulation (vs. 95% for the ipsilateral stimulation), which led to a maximum firing rate of 92 Hz during the ipsilateral stimulation vs. 21 Hz during the contralateral stimulation-demonstrating the spatial confinement in the radial direction. On the other hand, shifting the light beam about 200 μm along the axial direction led to an 80% reduction in light-evoked firing, demonstrating the spatial confinement in the axial direction.

### V. Eliciting Distinct Behaviors in the Deep Brain of Freely Moving Mice

The PRIME fiber's effectiveness for spatially confined, reconfigurable optogenetic neural modulation was demonstrated in freely moving mice (FIG. 19A). In this study, the PRIME fiber was chronically implanted in the superior colliculus (SC) (FIG. 19B), a seven-layered midbrain premotor region known to elicit distinct behaviors when different layers across the dorsal-ventral axis are activated. Specifically, activation of its intermediate or deep layer induces defensive freezing or flight reactions respectively (FIG. 19C). One week after the PRIME fiber implantation, individual Thy1-ChR2-YFP mice were placed in a 40×45 cm² open arena (Arena 1 in FIG. 30A) for habituation and free exploration. Upon their entry into a randomly pre-selected area, optogenetic stimulation with a 20-Hz frequency and 10-ms pulse width was applied for 2s, allowing the animals to have a non-zero initial speed. Then, three different axial sites along the same radial direction were stimulated, with two trials per site following the sequence of shallow, deep, combined, and then reverse (FIG. 19D). Stimulation at different depths induced discrete behavioral responses. Activation of the medial intermediate SC (SCmi) by the shallow stimulation elicited a relatively straight movement trajectory (FIG. 19E) and a reduction in speed, followed by recovery once the stimulation concluded (FIG. 19F). In contrast, activation of the medial deep SC (SCmd) by the deep stimulation resulted in a clockwise movement trajectory and a speed increase, which persisted for 1s after the conclusion of the stimulation (FIG. 19F and FIG. 30B). Simultaneous activation of the SCmi and SCmd by the combined stimulation replicated the behavioral responses to the activation of SCmd alone. However, the combined stimulation resulted in a more significant turning angle and a larger speed increase during and 1 s after the stimulation (FIG. 19F and FIG. 30B). Such behavior differences resulting from different axial stimulations (shallow, deep, combined) can be observed in recorded videos. The videos were captured from the same animal across three consecutive trials. The distinct behavior responses were consistent across animals and even evident at the single-trial level, including the differences in speed (FIG. 30B) and turning angle (FIG. 19E).

Differences in the behaviors elicited by optogenetic stimulation of different SC layers using the PRIME fiber can be further discerned at a more exquisite behavioral syllable level. To reveal such differences, an unsupervised behavior classification was conducted using B-SoiD26, which resulted in the identification of 48 distinct behavior syllables. To assess possible variations in the behavior syllable composition between different stimulation sites, the most frequently occurring behavior syllables from the shallow, deep, and combined stimulation were chosen as representative samples. Subsequently, the occurrence of these three representative syllables in each stimulation condition and in the control were quantified. As shown in FIG. 19G, Syllable 33, representing a slow forward movement, occurred most frequently in the shallow stimulation. Conversely, in the deep and combined stimulations, Syllables 27 and 28, both indicating clockwise turning movement albeit with varying degrees of intensity, dominated the behavioral repertoire.

The unique directivity and reconfigurability of the PRIME fiber in the radial illumination enabled one to explore the lateral heterogeneity of the SC in relation to behavior. Thus, three different illumination patterns were applied: medial, lateral, and combined within the intermediate layer of the SC (SCi). A week after the axially resolved optogenetic stimulation experiment described above, the same animals were introduced to a new arena (Arena 2 in FIG. 30A) for the radially resolved stimulation (FIG. 19H), where the medial stimulation targeted the same site as the prior shallow stimulation. Activation of different radial sites in the SCi led to distinct behavior phenotypes. Specifically, the medial stimulation induced a forward movement and a reduction in speed during stimulation (FIGS. 19H and 19J), resembling the observations in the shallow stimulation. In contrast, lateral stimulation induced a speed increase and the most pronounced turning during stimulation (FIGS. 191 and 19J, FIGS. 30D and 30E), while the combined stimulation showed a more moderate effect-a lower speed increase and subtle turning (FIGS. 191 and 19J).

Limited by the sample size, a greater variability in the movement patterns elicited by the medial and combined stimulations was observed (FIGS. 191 and K). However, the lateral stimulation induced speed increase and turning were consistent and robust, as evident by individual trials (FIGS. 191 and 30D and 30E). Such effect was also indicated by the prevalence of Syllable 27, corresponding to fast clockwise turning, in response to the lateral stimulation (FIG. 19K). The results, particularly regarding the extent of turning, align with prior findings that the SCi contains a discrete motor map responsible for coordinating 3D spatial head displacements, with observed variations in the displacement magnitude along the mediolateral axis. In addition, the different movement speeds during the medial and lateral stimulation indicate the disparity in the behaviors elicited by the two stimulation patterns. This study demonstrates the PRIME fiber's capability of high-precision spatial targeting, which enables the generation of distinct behavioral responses by selective activation of two adjacent sites that are separated by a mere fiber diameter of 160 μm.

To rule out the potential impact of other light-induced effects on behavior (e.g., the photothermal effect due to light absorption by blood or the light dump), the PRIME fiber was tested in wild-type mice. Following the same experimental protocols, 4-5 trials were collected per site per animal. The results show that the animals' movement trajectory (FIGS. 31A and 31D), speed (FIGS. 31C and 31F), and turning behavior (FIGS. 31B and 31E) remain overlapping and indistinguishable despite light illumination at different sites, suggesting that the different behavioral phenotypes indeed resulted from optogenetically evoked neural activities. Note that while statistical tests were not performed due to the limited sample size, the robust differences in the behavior elicited by different stimulation patterns, as observed at the single-trial level, still demonstrate the efficacy of the PRIME fiber for high-precision, large-scale, reconfigurable neural modulation in freely moving animals.

### Discussion

In Example 6, PRIME, a single-fiber technique for massively parallel optogenetic stimulation in the deep brain with rapid tuning, extensive axial coverage, and complete radial coverage, is introduced. Several features of the PRIME fiber underscore its potential for immediate neuroscience applications.

First, the PRIME fiber, sharing the same form factors and connectorization with conventional optical fibers, can be readily blended into the existing experimental paradigms in neuroscience. By following the standard practice in fiber-based optogenetics, we have applied the PRIME fiber has been applied in both head-fixed and freely moving mice. Also, it has been demonstrated that it can be bundled with commercial silicon probes for simultaneous optogenetics and electrophysiology. Moreover, it can be potentially deployed in a multi-fiber configuration to cover multiple transverse regions.

Second, the PRIME fiber enables the study of brain regions with closely spaced but functionally distinct subregions or layers. For example, it was demonstrated that emitting light to opposite side of the PRIME fiber along the mediolateral axis within a single SC layer elicited distinct movement patterns, despite a separation of only 160 μm. Such fine motor mapping is unachievable by existing fiber methods, even with multi-fiber implantation, due to constraints on the fiber diameter and invasiveness. Thus, the PRIME fiber may advance the understanding of brain regions associated with diverse motor functions, such as the basal ganglia and ventromedial hypothalamus.

Third, the PRIME fiber is well poised for scaling up and dissemination. The multicore fiber is commercially available and has been widely used for endoscopic imaging. The ultrafast laser direct writing, although new to neurotechnologies, has been widely used to fabricate fiber-optic devices for telecommunication and sensing applications. The laser-writing setup can fabricate a PRIME fiber within 5 minutes of laser processing time. Future implementation of the batch-produced strategy detailed in the Batch fabrication strategy section below (FIGS. 32A, 32B, 32C, 32D, 32E, AND 32F) will further reduce the time. The interrogation system (FIG. 27) consists of standard components, including a continuous-wave laser that is widely used for optogenetics and a SLM that has already been adopted for holographic optogenetics. In applications where multi-site illumination is not required, such as ChR2-assisted circuit mapping or light-based motor mapping, a Galvo scanner can be used in place of the SLM (FIG. 27B).

Fourth, the PRIME fiber is expected to enable close-loop investigation of functional hierarchy and coordination across brain regions by dynamically adapting the reconfigurable optogenetic illumination pattern to experimental observations in the same animal. Also, the reconfigurability may enable the investigation of information flow and computation by selectively modulating the activity at each node of a circuit. Conveniently, it also allows a greater degree of freedom to compensate for errors associated with the implantation surgery.

Refinements to the PRIME fiber can further advance its abilities and adoption. First, the current size of the PRIME fiber (160 μm), although comparable to commonly used optical fibers, is larger than electrical recording probes (<100 μm). Reducing the diameter will mitigate tissue damage and facilitate multi-fiber implantation. This can be achieved by increasing the index contrast and thus the core density, as suggested by a numerical study. Second, the current reconfiguration rate of the illumination pattern (60 Hz) is limited by the modulation speed of the SLM. It falls short for the sub-millisecond temporal neural modulation in circuit dynamics studies. This can be addressed by using a liquid crystal-based SLM with 1.6-kHz tuning speed or a deformable-mirror-device-based SLM, whose speed can reach 30 KHz at the expense of light power efficiency. Third, the current PRIME fiber is based on glass, which is stiffer than soft tissue and poses a potential risk of brain damage in freely moving animals. By applying ultrafast laser direct writing in transparent media, the PRIME technique can be extended to non-glass fibers, such as those based on polymer and hydrogel. Having lower bending stiffness, these materials can help mitigate deformation-induced tissue damage. With further improvement and optimization, the PRIME technique is poised to empower neuroscientists in exploring a diverse range of hypotheses concerning how spatiotemporal patterns of neural activity underpin behavior. This process will advance the understanding of neural codes.

### Methods

### Fabrication of the PRIME Fiber

The multicore fiber was purchased from Fujikura (FIGH-016-160S). Examination of the fiber under an optical microscope showed 1,460 light-guiding cores distributed within a 150-μm circle, with an overall diameter of 160 μm. Grating light emitters were inscribed in individual cores using the ultrafast laser direct writing setup described herein and illustrated in FIG. 25. Then, the fiber was cleaved ˜500 μm away from the laser-inscribed region under a stereo microscope. The cleaved fiber was dip-coated with a graphite-epoxy mixture (20 wt %, graphite nanopowder: 400 nm-1.2 μm, US Research Nanomaterials, Inc.; Epoxy: AA-BOND F112, Atom adhesives) and thermally cured in an oven at 90° C. for 30 minutes. The cured tip is laser-machined into a tapered shape by the same setup in the Ultrafast laser direct writing of the fiber grating emitter section below and FIG. 25. Finally, the fiber is cut into the desired length, fixed into a 1.25-mm ceramic ferrule (bore size: 170 μm, Precision Fiber Products, Inc.) with the same epoxy, and polished for a flat surface with an automatic fiber polisher (NOVA Optical Polishing System, KrellTech).

### Characterization of the PRIME Fiber

The grating structure was imaged using a brightfield light microscope (: Ultrafast laser direct writing of the fiber grating emitter section and FIG. 25). The structure of the laser-inscribed nano-void was exposed by cleaving the fiber within the grating section and then imaged using a scanning electron microscope (Thermofisher Quattro S ESEM). The emission profiles at 450 nm, 532 nm, and 660 nm were measured by immersing the laser-inscribed region in a solution containing fluorescein (MilliporeSigma), Texas red (ThermoFisher), and Alexa Fluor 680 (ThermoFisher). The other end of the fiber was connected to the characterization setup shown in FIG. 27. A high magnification zoom lens system was used to capture the emission images from the side with corresponding filters (LP500+532 nm notch filter for fluorescein and Texas red; 700/40 for Alexa Fluor 680). Based on these images, the emission profiles (FIG. 12B) and diffraction angles (FIGS. 12D and E) were obtained. The emission efficiency was characterized by the setup described in the Characterization of the fiber grating emission section below and FIG. 26. The parameters used to plot the theoretical curves in FIG. 12E are described below. First, the refractive index of the cladding, η_{clad}, is set to 1.4656, 1.4607, and 1.4563 for 450 nm, 532 nm, and 660 nm, respectively. Then, the refractive index of the core is calculated via n_{core}=(NA²-n_{clad}²)^{0.5}, where NA=0.4 is the numerical aperture of the image guide. The η_{eff} is derived by solving the fiber modes under the assumption of a circular cross-section. Although the fiber supports multiple modes at the three wavelengths, only the fundamental mode was chosen in this work due to its high excitation probability by a uniform and on-axis illumination. The ambient refractive index, ηₐ, is set to 1.33.

### Interrogation System

The patch cable was made from a 215-μm-diameter multicore fiber (FIGH-03-215S, Fujikura). The larger diameter ensures a full coverage of the cores in the PRIME fiber within the manufacture tolerance of the fibers and ferrules. One end of the fiber was fixed in a FC/PC connector (30230C1, Thorlabs), and the other end was fixed in a 1.25 mm ceramic ferrule (CFX230-10, Thorlabs) secured by an aluminum tube (92510A381, McMaster-CARR). A 900-μm furcation tube (FOOFR900HO, Fiber Instruments Sales) was used to increase the mechanical strength of the patch cable. A groove was laser-marked on the face of the FC/PC connector and on the aluminum tube to label the orientation. Details of the interrogation system are described herein and illustrated at FIG. 27A. The SSIM between the input and transmitted patterns shown in FIG. 16D was calculated by the Complex-Wavelet Structural Similarity Index method. The profiles of the spatially reconfigurable emission in FIG. 13C were captured by the same setting described in the section of “Characterization of the PRIME fiber”. The dynamic results in FIG. 13D were recorded by two photodiodes (PDA25K2, Thorlabs) placed at the two sides of the fiber.

### Integration of the PRIME Fiber and an Electrode Probe

The multi-electrode array probe was obtained from NeuroNexus (A2x16-3 mm-65-500-177-A32). The probe has two shanks (500 μm apart), each of which contains 16 electrodes with an interval of 65 μm. Based on the spatial coverage and the total length of the NeuroNexus probe, a PRIME fiber with an emission range of 1.5 mm and a total length of 70 mm was fabricated. The PRIME fiber was mounted on a 3D translational stage and carefully placed onto the electrode probe under a microscope. Upon reaching the desired position, the fiber was secured by a UV-cured electronic adhesive (NEA 426 121, Norland) on the electrode probe. The ferrule was then secured by an epoxy (Gorilla 5-minute epoxy) to increase the mechanical strength.

### Multi-Electrode Recording

The PRIME fiber was interrogated with the setup described in FIG. 16 and FIG. 26A. The electrical signal from the multi-electrode probe was sampled at 30 kHz by a data acquisition system (SpikeGadgets Main Control Unit). A control unit (Bpod state machine) was used to trigger the laser and synchronize the PRIME fiber interrogation system and the electrode probe recording unit. A 1-ms-duration light pulse was delivered into the fiber every 4s, and 20 repetitions were performed for each illumination condition. The output power of the PRIME fiber at each site was 100-200 μW. The local field potential (LFP) signal was extracted using a low pass filter (cut-off frequency: 100 Hz), and a 60-Hz notch filter (3-dB bandwidth: 5 Hz) was used to eliminate electrical line noise.

The current source density (CSD) was estimated by the inverse CSD (iCSD) method with the averaged LFP:

$$C = {F}^{- 1} \cdot Φ$$

$${F}_{ji} = \frac{{h}^{2}}{2 σ} [ \begin{matrix}\sqrt{{( j - i )}^{2} + {( \frac{R}{h} )}^{2}} - \vert j - i \vert\end{matrix} ]$$

where C=[C₁ C₂ . . . . C_{N}] and Φ=[Φ₁ Φ₂ . . . Φ_{N}]^{T} are the CSD and averaged LFP values at the electrode locations with the total electrode number N=16, h is the spacing between adjacent electrodes (65 μm), R is the radius of the electrode (50 μm), and σ is the conductivity of the extracellular medium (0.3 S.m⁻¹). The CSD plots in FIG. 17D were interpolated 4 times and smoothed along the depth dimension for visualization purpose.

Single unit analysis was carried after threshold-based extraction of spikes using SpikeGadgets acquisition software (Trodes). Manual clustering was performed using MClust on pseudo tetrodes (8×4 pseudo-tetrodes). Clusters with an L-ratio less than 0.5 and an isolation metric above 15 were kept for further analysis. Light-responsive units were defined as an increase in firing rate following the light stimulation (0-10 ms time window) equivalent or superior to 50 times of the baseline firing rate. Light-responsive units from both shanks and from independent recordings were combined for depth- and direction-dependent analysis.

### Animals

Adult male or female Thy1-ChR2-YFP mice (Jackson Labs, 007612) were used under the protocol approved by the Institutional Animal Care and Use committee at Washington University in St. Louis, in accordance with National Institutes of Health's regulations. Mice were singly housed, maintained with a 12 h: 12 h light: dark cycle with ad libitum food and water. Surgeries were performed when the animals were 14 weeks old.

### Multi-Electrode Array Recording Surgery

Thy1-ChR2-YFP mice were anesthetized with isoflurane, and their head and back fur was shaved. Mice were then placed in a stereotactic frame with a heating pad, and a sagittal incision was made down the center of the scalp to expose the cranium. A metal head bar was attached to the skull above the lambda using Vitrebond after roughening the skull. A 2×2 mm² cranial window was made, with its center aligned over the secondary motor cortex (AP: 2.6 mm, ML: 1 mm). On the opposite hemisphere of the cranial window, a different craniotomy was made. One end of a grounding wire was inserted into this craniotomy, while the other end was secured to the head bar using Vitrebond. The animal was then carefully transferred from the stereotactic frame to a head-fixed station located within a metal enclosure, providing sensory isolation and filtering of electrical fields. After head fixation, the constructed optrode was slowly inserted into the brain through the cranial window using a motorized arm (Thorlabs Z825B) until all electrodes were situated beneath the brain surface. Throughout the experiment, anesthesia was maintained using isoflurane (1 L/min O₂ with 0.5-1% isoflurane).

### Chronic Implantation of the PRIME Fiber

Animals were anesthetized using isoflurane and placed in a stereotaxic apparatus. A small craniotomy was performed using a dental drill above the right superior colliculus (AP:−3.7 mm, ML: 0.35). Similar to conventional fiber implantation, the PRIME fibers were inserted 3 mm below the brain surface and sealed in place using Metabond, Vitrebond, and dental acrylic. Behavioral testing was started 7-10 days after surgery to allow mice to recover.

### Open-Field Behavior Test

Two adult mice (A1 and B2) were individually placed in a square arena (45×40 cm²) for depth emission testing. Behavior protocol was implemented using a Bpod behavioral controller (Sanders and Kepecs) and the optogenetic pulses were generated using a PulsePal device. An overhead camera, under the control of a custom-written Bonsai script, recorded trial-by-trial activities. Prior to the initiation of any optogenetic stimulation trial, mice were given a 5-minute period to explore the arena. Once the animal entered a randomly preselected subregion approximately 8×8 cm², as defined within the Bonsai software, optogenetic stimulations with 20-Hz frequency and 10-ms pulse width were triggered for 2s. The output light power from the PRIME fiber at each site was 3-5 mW. The first testing session contained six stimulation trials, encompassing three pseudorandom emission patterns, with a 120-s interval between stimulation trials. A week after the first session, an individual mouse was placed into an arena (a 50x30 cm² rectangle or a triangle with 55-cm, 35-cm, and 35-cm sides) for testing radial emission with an identical testing protocol.

### Behavior Analysis

Animals' poses were extracted post hoc from the recorded videos using DeepLabCut, a machine-learning package that can perform tracking of animal features. Data were analyzed using a network trained on 140 frames of recorded behavior run for 100,000 training iterations. The network was trained to detect the tip of the nose, the center of the head, the neck, two points along the spine, and the base of the tail. The centroid of the mouse was calculated by averaging these six points. DeepLabCut-labeled points with a predicted accuracy below 0.6 were excluded from the analysis. Speed was determined by dividing the frame rate by the displacement of the centroid, which was then smoothed over a 1/3-s time window. To determine the orientation of the mouse, a vector from the neck to the nose was generated for each frame, with the direction of this vector indicating the orientation of the animal. The orientation at which the optogenetic stimulation started was normalized to be at 0 by subtracting an averaging orientation of the 100-ms time window before stimulation onset from the current orientation.

To analyze the behavioral syllable compositions under various illumination patterns, the extracted poses were inputted into the unsupervised behavioral classification algorithm, B-SOiD26. A generalized B-SOiD model was created by combining data from 3 mice. Employing nonlinear embedding, the algorithm transformed the initial 64-dimensional data into a 6-dimensional UMAP space. Subsequently, 48 behavioral clusters were identified and utilized to train the random forest classifier. The trained classifier was then applied to assign behavioral labels to each stimulation pattern. The algorithm had no access to information beyond the spatiotemporal relationship among the 6 extracted poses.

### Histology

Mice were anesthetized and transcardially perfused with 0.9% saline followed by 4% paraformaldehyde in phosphate buffer saline (PBS). The brains were post-fixed in 4% paraformaldehyde solution overnight at 4° C., and then prepared for sectioning by washing with PBS. The brain tissue was embedded with 2% agarose gel, mounted on the vibratome stage (Leica VT 1000S), and sliced into 200-μm-thick coronal sections. The slices were mounted on slides with an antifade mounting medium (Southern Biotech DAPI Fluoromount-G). The images were captured on Leica THUNDER Imager 3D Tissue with 5× objective.

### Ultrafast Laser Direct Writing of the Fiber Grating Emitter

As shown in FIG. 25A, the setup for ultrafast laser direct writing uses a frequency-doubled femtosecond (fs)-pulsed laser (Coherent Monaco 1035; output wavelength, 517 nm; pulse duration, ˜270 fs). The laser power and polarization are controlled using the combination of a half-wave plate (N/2, EKSMA Optics 467-4205) and a polarizing beam splitter (PBS, EKSMA Optics 435-1124D). Then, the power-regulated beam passes through a beam expander (L1+L2, Edmund Optics 87-563) to adjust its diameter and thus the effective numerical aperture (NA) for light focusing. By following a slit-beam shaping method, the circular laser spot is reshaped into a line for laser inscription of the nano-void-based fiber grating. By adjusting the orientation of a mechanical slit (Thorlabs VA100CP) fixed on a rotation mount, gratings with different morphologies can be created (FIG. 25B). In this Example, a slit size of 400 μm was chosen to best match the nano-void and core sizes. Then, the line-shaped beam is projected onto the entry pupil of an objective lens (Obj1, Zeiss 100× oil immersion) through a 4-f imaging setup (L3, L4: f=125 mm, Thorlabs AC254-125-A-ML) and subsequently focused into the fiber. The fiber is mounted on a customized four-axis motorized stage, which allows 3D linear translation (Physik Instrumente A-311 and V-308) as well as rotation along the fiber axis (home-built). A fused silica refractive index matching liquid (Cargille Laboratories) is applied between the fiber and the objective to eliminate the optical aberration induced by the cylindrical side surface of the fiber. Alignment of the laser focus and the fiber is achieved using an inline transmissive bright-field microscope, where a 465-nm LED (Thorlabs LED465E) positioned beneath the fiber is used as the illumination source. The fiber image is projected onto a CMOS camera (Cam1: The Imaging Source DFK23UM021) through the same objective (Obj1) and a tube lens (L5, f=200 mm, Thorlabs AC254-200-A-ML). The laser and imaging paths are separated using a short-pass dichromic mirror (DM, cut-off: 505 nm, Thorlabs DMSP505R).

### Characterization of the Fiber Grating Emission

The emission properties of the fiber grating were characterized using laser light at three different wavelengths across the visible spectrum (i.e., 450 nm, 532 nm, and 660 nm). As shown in FIG. 26, each of the three continuous-wave (CW) laser beams was coupled into the same single-mode fiber (Thorlabs S405-XP) and then collimated using the same achromatic fiber collimator (L2, Thorlabs C40FC-A). This configuration allows one to switch wavelengths without affecting the system alignment. The collimated beam is focused into a grating-inscribed core of the PRIME fiber through an objective lens (OBJ, Olympus PLN 20×). A reflective bright-field microscope was constructed to assist the alignment, and the PRIME fiber was mounted on a 3D translation stage for position adjustment. For characterization of the emission profile, the PRIME fiber was immersed in a fluorescent solution, and a telescope system (Thorlabs MVL6X12Z and MVL20A and The Imaging Source DFK 33UX249) equipped with corresponding filters was used to capture pictures of both the side and the cross-section views (see Methods for details). For measurement of the emission efficiency, a customized container consisting of an ellipsoidal reflector (Edmund Optics 68-797) and a glass plate (McMaster 8476K14) was built to fully capture the side-emitted light. The container was filled with water to mimic the refractive index of the brain tissue. The PRIME fiber was immersed in water, and the grating emitter was placed near one of the foci of the ellipsoid. One power meter (P1) was placed near the fiber's distal end to capture the unemitted light, while another power meter (P2) was placed at the other foci of the ellipsoid to collect the emitted light. The emission efficiency (n) can be calculated by η=P2/(r_{ER}P1+P2), where r_{ER}=0.9 is the reflectivity of the ellipsoidal reflector.

### Interrogation System for the PRIME Fiber

Two interrogation systems for the PRIME fiber are introduced: holographic pattern projection and core-to-core scanning (FIG. 27). In the holographic system (FIG. 27A), a spatial light modulator (SLM, Thorlabs EXULUS-HD2) is used to generate the hologram. A collimated CW laser beam is expanded (L1: f=25.4 mm L2: f=100 mm, Thorlabs LA1951-A-ML and LA1509-A-ML) before illuminating the SLM to utilize all pixels. The laser polarization is aligned with the SLM's optical axis using a half-wave plate (Thorlabs WPHSM05-473) to maximize the modulation efficiency. A computer-generated hologram (CGH) pattern calculated by the Gerchberg-Saxton algorithm is displayed on the SLM. A blaze grating phase pattern is superimposed on the hologram to direct it to the first-order diffraction. Then, the phase-modulated laser beam is Fourier transformed by a lens (L3, f=300 mm, Thorlabs ACT508-300-A-ML) where the zero-order, non-modulated portion is blocked by an iris (Thorlabs SM1D12D). The spatially filtered hologram is projected onto the fiber input through a 4-f system (L4: f=100 mm, Thorlabs AC508-100-A-ML; Obj: Olympus PLN 20×). For applications where only single-site emission is needed, the core-to-core scanning system can be used instead to leverage the relatively low cost and high speed (>1 kHz) of a Galvo scanner. In this system (FIG. 27B), a 2D Galvo scanner can be used to steer the laser beam. Then, a lens pair (L6 and L7, Thorlabs AC254-100-A-ML) relays the beam to the entrance pupil of the objective lens for coupling into the PRIME fiber. In both configurations, a bright-field microscope is constructed to assist the alignment as described above.

### Batch Fabrication Strategy

A batch-fabrication strategy for the PRIME fiber is proposed (FIG. 32). In the field of fiber sensing and telecommunications, it is feasible to fabricate hundreds to thousands of gratings distributed along hundreds of meters of fiber using ultrafast laser direct writing. By adopting this approach, one can fabricate multiple emitting sections with a single-step fabrication. Then, the fiber is cleaved into multiple probes with desired lengths. These probes can be assembled to fabricate light-absorbing coating through dip coating and thermal curing. Finally, they are packaged into the ferrules and polished together.

### Example 7-Deep-Tissue Large-Field-of-View Multi-Scale Fiber-Optic Imager

Optical imaging has been widely used in basic and translational biomedicine owing to its scalable spatial resolution and rich image contrast. However, existing optical imaging techniques are divided into two distinct categories, with a large gap in between. On the one hand, optical microscopy offers cellular or even sub-cellular resolution, but the penetration is limited to 1-2 millimeters. On the other hand, taking advantage of diffuse photons, optical tomography extends the penetration to a few centimeters, but the resolution is macroscopic (a few millimeters). To break the penetration-resolution tradeoff and to further extend the penetration depth of optical imaging, minimally invasive endoscopic approaches have been widely adopted to gain direct access to the deep tissue. However, the field of view provided by existing techniques is limited to a small region near the tip of the endoscopic probe, resulting in a very limited Minimize spatial coverage. Increasing the number of probes only scales up the coverage linearly and often leaves gaps between the individual fields of view.

Example 7 describes a method for minimally invasive optical imaging in deep tissues in vivo with side-emission-collection fiber implant(s). It includes the fiber implant, an interrogation system, and an imaging reconstruction method (FIG. 24).

The fiber implant used in Example 7 is capable of dynamic and reconfigurable light emission and collection along and around the fiber. Single or multiple such fibers can be implanted into the subject to be imaged, either an animal or a human. The number and spatial distribution of the fiber implants are determined by the application requirements, including but not limited to spatial resolution, imaging location, and spatial coverage. The imaging procedure is as follows. First, light is coupled into the fiber(s) and emitted at designated depth(s) and angular direction(s). The emitted light interacts with the tissue through scattering, absorption, and/or re-emission. The scattering and absorption only change the directivity and intensity of the emitted light. In contrast, the re-emission can change the wavelength of the light via mechanisms including but not limited to fluorescent emission, Raman scattering, Brillouin scattering, and harmonic generation. After a series of light-tissue interactions during the photon propagation, the scattered, non-absorbed, and/or re-emitted light is collected by the fiber implants at designated depth(s) and angular direction(s). The light emission and collection can be within the same fiber (intra-fiber) and/or among different fibers (inter-fiber). At this point, a data pair, including a specific light emission pattern (location and directivity) and its corresponding light collection pattern, is acquired. Then, the process is iterated for different light emission-collection patterns until the desired number of data pairs is achieved for image reconstruction. This process can be repeated with light at different wavelengths for ratiometric measurements and/or normalization.

Besides the fiber implant, an interrogation system is used to launch light into and collect light from the individual cores of the fiber. The launching part selectively couples the light into the designated light-guiding cores. It can be done via a point-by-point scanning system with galvo scanners or a MEMS mirror, or a pattern projection setup with a spatial light modulator (SLM) or a digital mirror device (DMD). Either incoherent or coherent light sources can be used. The collection part can be a CMOS or CCD camera to encode the light collection from different sites into different pixels.

The acquired data pairs will be fed into a computational model for imaging reconstruction. Different imaging scales can be achieved with different reconstruction models. For microscopic imaging, wave-based models such as single-layer or multi-layer Born models can be implemented. For mesoscopic/macroscopic imaging, diffuse-optical methods such as the diffuse-approximation model or Monte-Carlo simulation can be used. The imaging contrast is determined by the type of collected light. The light modulated by tissue scattering and/or absorption can be used to reconstruct the distribution of the refractive index and/or the concentrations of optical absorbers. The re-emitted light can be used to reconstruct the distribution of fluorophores (fluorescence) and biomolecules (Raman), mechanical properties (Brillouin), and/or structural information (harmonic generation).

The minimally invasive optical imaging method utilizes the distributed light emission and collection of the fiber implant to form a 3D excitation-detection array for microscopic and/or tomographic image formation in deep tissues in vivo, which would otherwise require at least 10 times more conventional optical fibers that can only perform light emission and collection at the tip. The device described in Example 7 significantly reduces the invasiveness and makes the implantation of a high-density array practically possible. Furthermore, multi-scale imaging spanning from microscopy (˜μm resolution) to tomography (˜mm resolution) can be achieved by the design of the fiber array and the implemented computational model, which provides excellent flexibility and adaptability for different applications.

What is claimed is:

1. A fiber-optic bi-directional interface device, comprising a multi-core optical fiber comprising a plurality of light-guiding cores, each light-guiding core comprising opposed proximal and distal ends, the proximal end configured to receive light from a light source, wherein:
a. a first portion of the plurality of light-guiding cores further comprises light emission modifications configured to direct light propagating distally along the light-guiding cores in a laterally outward or sideways direction relative to the propagation axis of the light-guiding core; and
b. a second portion of the plurality of light-guiding cores further comprises light collection modifications configured to receive light produced by a source positioned laterally outwards or sideways relative to the propagation axis of the light-guiding core and direct the received light proximally.

2. The device of claim 1, wherein the light emission modifications and the light collection modifications are distributed along an emission/collection span extending a predetermined proximal-distal distance along the light-guiding cores.

3. The device of claim 1, wherein the light emission modifications are selected from the group consisting of photonic crystals, fiber Bragg gratings (FNGs), and any combination thereof.

4. The device of claim 1, wherein the light collection modifications are selected from the group consisting of total internal reflection (TIR) mirrors, multi-layer gradient filters, and any combination thereof.

5. The device of claim 1, further comprising a light trap positioned at the distal ends of the plurality of light-guiding cores.

6. A system to conduct a light-based procedure, the system comprising a fiber-optic bi-directional interface device optically coupled to an interrogation assembly, wherein:
a. the fiber-optic bi-directional interface device comprises a multi-core optical fiber comprising a plurality of light-guiding cores, each light-guiding core comprising opposed proximal and distal ends, the proximal end configured to receive light from a light source, wherein:
i. a first portion of the plurality of light-guiding cores further comprises light emission modifications configured to direct light propagating distally along the light-guiding cores in a laterally outward or sideways direction relative to the propagation axis of the light-guiding core; and
ii. a second portion of the plurality of light-guiding cores further comprises light collection modifications configured to receive light produced by a source positioned laterally outwards or sideways relative to the propagation axis of the light-guiding core and direct the received light proximally; and
b. the interrogation assembly comprises:
i. the light source optically coupled to the proximal ends of the plurality of light-guiding cores, wherein the light source is configured to produce and direct light selectively into the proximal end of one or more light-guiding cores from the first plurality in a pre-determined pattern;
ii. a light detector optically coupled to the proximal ends of the plurality of light-guiding cores, wherein the light detector is configured to receive and detect light propagating from the proximal end of one or more light-guiding cores from the second plurality.

7. The system of claim 6, wherein the light emission modifications and the light collection modifications are distributed along an emission/collection span extending a predetermined proximal-distal distance along the light-guiding cores.

8. The system of claim 6, wherein the light emission modifications are selected from the group consisting of photonic crystals, fiber Bragg gratings (FNGs), and any combination thereof.

9. The system of claim 6, wherein the light collection modifications are selected from the group consisting of total internal reflection (TIR) mirrors, multi-layer gradient filters, and any combination thereof.

10. The system of claim 6, further comprising a light trap positioned at the distal ends of the plurality of light-guiding cores.

11. The system of claim 6, further comprising a launching element configured to selectively transmit a portion of light produced by the light source into the proximal ends of one or more light-guiding cores in the predetermined pattern, wherein the launching element comprises one of:
a. a spatial light modulator optically coupled between the light source and the proximal ends of the plurality of light-guiding cores;
b. a digital mirror device (DMD) optically coupled between the light source and the proximal ends of the plurality of light-guiding cores;
c. a galvo scanner operatively coupled to the light source to scan the light source point by point to the one or more light-guiding core; or
d. a MEMs mirror optically coupled between the light source and the proximal ends of the plurality of light-guiding cores.

12. The system of claim 6, wherein the pre-determined pattern is selected from a depth-selective light pattern, a spatially patterned light pattern, a large-volume illumination light pattern, and any combination thereof.

13. The system of claim 6, wherein the light-based procedure selected from the optogenetic stimulation, photometry, microscopic imaging, tomographic imaging, and any combination thereof.

14. A method of producing a fiber-optic bi-directional interface device configured to transmit and receive light oriented perpendicular to a device light propagation axis, the method comprising:
a. providing a multi-core optical fiber comprising a plurality of light-guiding cores, each light-guiding core comprising opposed proximal and distal ends;
b. for a first portion of the light-guiding cores, delivering a series of slit-shaped laser pulses from a femto laser to a selected interior region of the light-guiding core to produce a series of cavities within a proximal-distal section of the light-guiding core to form a fiber grating within the fiber, wherein the fiber grating is configured to direct light propagating distally along the light-guiding cores in a laterally outward or sideways direction relative to the propagation axis of the light-guiding core; and
c. for a second portion of the plurality of light-guiding cores, delivering a series of laser pulses from a femto laser to a selected interior region of the light-guiding core to produce a rectangular-shaped cavity within a proximal-distal section of the light-guiding core to form a total internal reflection (TIR) mirror configured to receive light produced by a source positioned laterally outwards or sideways relative to the propagation axis of the light-guiding core and direct the received light proximally along the light-guiding core.
